SEQUENCE LISTING (32 sequences)

SEQ ID NO: 1 - protein, 19 aa, synthetic construct
  MOD_RES 4: note = Glutamic acid at position 4 has a protected side chain (-CO2tBu)
  MOD_RES 5: note = Tyrosine at position 5 has a protected side chain (-OtBu)
  MOD_RES 8: note = Glutamic acid at position 8 has a protected side chain (-CO2tBu)
  MOD_RES 12: note = Serine at position 12 has a protected side chain (-OtBu)
  MOD_RES 13: note = Serine at position 13 has a protected side chain (OtBu)
  MOD_RES 19: note = Serine at position 19 has a protected side chain (-OtBu)
  MOD_RES 19: note = Serine at position 19 is amidated with a hydrophilic linker (Rink-(AEEA)6-NH2)
AFIEYLLEGGPSSGAPPPS

SEQ ID NO: 2 - protein, 19 aa, synthetic construct
  MOD_RES 19: note = Serine at position 19 is amidated as a C-terminal primary amide
AFIEYLLEGGPSSGAPPPS

SEQ ID NO: 3 - protein, 10 aa, synthetic construct
  MOD_RES 3: note = Serine at position 3 has a protected side chain (-OtBu)
  MOD_RES 4: note = Serine at position 4 has a protected side chain (-OtBu)
  MOD_RES 10: note = Serine at position 10 has a protected side chain (-OtBu)
  MOD_RES 10: note = Serine at position 10 is amidated with a hydrophilic linker (Ramage-(AEEA)10-NH2)
GPSSGAPPPS

SEQ ID NO: 4 - protein, 10 aa, synthetic construct
  MOD_RES 3: note = Serine at position 3 has a protected side chain (-OtBu)
  MOD_RES 4: note = Serine at position 4 has a protected side chain (-OtBu)
  MOD_RES 10: note = Serine at position 10 is amidated as a C-terminal primary amide
GPSSGAPPPS

SEQ ID NO: 5 - protein, 10 aa, synthetic construct
  MOD_RES 3: note = Serine at position 3 has a protected side chain (-OtBu)
  MOD_RES 4: note = Serine at position 4 has a protected side chain (-OtBu)
  MOD_RES 10: note = Serine at position 10 has a protected side chain (-OtBu)
  MOD_RES 10: note = Serine at position 10 is amidated with a hydrophilic linker (Rink-(AEEA)2-NH2)
GPSSGAPPPS

SEQ ID NO: 6 - protein, 8 aa, synthetic construct
  MOD_RES 1: note = Phenylalanine at position 1 is -Fmoc protected
  MOD_RES 3: note = Glutamine residue at position 3 has a protected side chain (-CONH-trityl)
  MOD_RES 4: note = Tryptophan at position 4 has a protected side chain (-NBoc-)
  MOD_RES 8: note = Glycine at position 8 is esterified with a hydrophilic linker (HMPA-(AEEA)10-NH2)
FVQWLIAG

SEQ ID NO: 7 - protein, 8 aa, synthetic construct
  MOD_RES 1: note = Phenylalanine at position 1 is -Fmoc protected
  MOD_RES 3: note = Glutamine at position 3 has a protected side chain (-CONH-trityl)
  MOD_RES 4: note = Tryptophan at position 4 has a protected side chain (-NBoc-)
FVQWLIAG

SEQ ID NO: 8 - protein, 8 aa, synthetic construct
  MOD_RES 1: note = Phenylalanine at position 1 is -Fmoc protected
  MOD_RES 3: note = Glutamine at position 3 has a protected side chain (-CONH-trityl)
  MOD_RES 4: note = Tryptophan at position 4 has a protected side chain (-NBoc-)
  MOD_RES 8: note = Glycine at position 8 is esterified with a hydrophilic linker (HMPB-(AEEA)10-NH2)
FVQWLIAG

SEQ ID NO: 9 - protein, 7 aa, synthetic construct
  MOD_RES 1: note = Aspartic acid at position 1 is -Fmoc protected
  MOD_RES 1: note = Aspartic acid at position 1 has a protected side chain (-CO2tBu)
  MOD_RES 2: note = Lysine at position 2 has a protected side chain (-NH-Boc)
  MOD_RES 5: note = Glutamine at position 5 has a protected side chain (-CONH-trityl)
  MOD_RES 6: note = Lysine at position 6 is chemically modified by conjugation of the epsilon-amino group of the Lys side chain with -(AEEA)2-gamma glutamic acid(OtBu)-CO-(CH2)18-CO2tBu
  MOD_RES 7: note = Alanine at position 7 is esterified with a hydrophilic linker (HMPB-(AEEA)10-NH2)
DKIAQKA

SEQ ID NO: 10 - protein, 7 aa, synthetic construct
  MOD_RES 1: note = Aspartic acid at position 1 is -Fmoc protected
  MOD_RES 2: note = Lysine at position 2 has a protected side chan (-NH-Boc)
  MOD_RES 1: note = Aspartic acid at position 1 has a protected side chain (-CO2tBu)
  MOD_RES 5: note = Glutamine at position 5 has a protected side chain (-CONH-trityl)
  MOD_RES 6: note = Lysine at position 6 is chemically modified through conjugation to the epsilon-amino group of the Lys side-chain with (AEEA)2-gamma glutamic acid(OtBu)-CO-(CH2)18-CO2tBu
DKIAQKA

SEQ ID NO: 11 - protein, 14 aa, synthetic construct
  MOD_RES 1: note = Tyrosine at position 1 is -Boc protected
  MOD_RES 1: note = Tyrosine at position 1 has a protected side chain (-OtBu)
  NON_STD 2: note = X at position 2 is nonnaturally occurring amino acid 2-Aminoisobutyric Acid (Aib)
  MOD_RES 3: note = Glutamic acid at position 3 has a protected side chain (-CO2tBu)
  MOD_RES 5: note = Threonine at position 5 has a protected side chain (-OtBu)
  MOD_RES 7: note = Threonine at position 7 has a protected side chain (-OtBu)
  MOD_RES 8: note = Serine at position 8 has a protected side chain (-OtBu)
  MOD_RES 9: note = Aspartic acid at position 9 has a protected side chain (-CO2tBu)
  MOD_RES 10: note = Tyrosine at position 10 has a protected side chain (-OtBu)
  MOD_RES 11: note = Serine at position 11 has a protected side chain (-OtBu)
  NON_STD 13: note = X at position 13 is nonnaturally occurring amino acid 2-Aminoisobutyric Acid (Aib)
  MOD_RES 14: note = Leucine at position 14 is esterified with a hydrophilic linker (HMPB-(AEEA)10-NH2)
YXEGTFTSDYSIXL

SEQ ID NO: 12 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic linker (HMPB-(AEEA)10-NH2)
YAEG

SEQ ID NO: 13 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Gutamic acid at position 3 has a protected side chain (-CO2tBu)
YAEG

SEQ ID NO: 14 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic linker (HMPB-(AEEA)4-NH2)
YAEG

SEQ ID NO: 15 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic linker (HMPB-(AEEA)2-NH2)
YAEG

SEQ ID NO: 16 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic linker (HMPB-(AEEA)6-NH2)
YAEG

SEQ ID NO: 17 - protein, 4 aa, unidentified
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic linker (HMPA-(AEEA)10-NH2)
YAEG

SEQ ID NO: 18 - protein, 4 aa, synthetic construct
  MOD_RES 1: note = Tyrosine at position 1 is -Boc protected
  MOD_RES 1: note = Tyrosine at position 1 has a protected side chain (-OtBu)
  MOD_RES 2: note = Alanine at position 2 is alpha-methylated (Aib)
  MOD_RES 3: note = Glutamic acid at position 3 has a protected side chain (-CO2tBu)
  MOD_RES 4: note = Glycine at position 4 is esterified with a hydrophilic side chain (HMPA-(AEEA)2-NH2)
YAEG

SEQ ID NO: 19 - protein, 10 aa, synthetic construct
  MOD_RES 3: note = Serine at position 3 has a protected side chain (-OtBu)
  MOD_RES 4: note = Serine at position 4 has a protected side chain (-OtBu)
  MOD_RES 10: note = Serine at position 10 has a protected side chain (-OtBu)
  MOD_RES 10: note = Serine at position 10 is amidated with a hydrophilic linker (Rink-(AEEA)6-NH2)
GPSSGAPPPS

SEQ ID NO: 20 - protein, 18 aa, unidentified
  MOD_RES 1: note = Phenylalanine at position 1 is -Fmoc protected
  MOD_RES 3: note = Glutamine at position 3 has a protected side chain (-CONH-trityl)
  MOD_RES 4: note = Tryptophan at position 4 has a protected side chain (-NBoc-)
  MOD_RES 11: note = Serine at position 11 has a protected side chain (-OtBu)
  MOD_RES 12: note = Serine at position 12 has a protected side chain (-OtBu)
  MOD_RES 18: note = Serine at position 18 has a protected side chain (-OtBu)
  MOD_RES 18: note = Serine at position 18 is amidated with a hydrophilic linker (Rink-(AEEA)6-NH2)
FVQWLIAGGPSSGAPPPS

SEQ ID NO: 21 - protein, 18 aa, synthetic construct
  MOD_RES 3: note = Glutamine at position 3 has a protected side chain (-CONH-trityl)
  MOD_RES 4: note = Tryptophan at position 4 has a protected side chain (-NBoc-)
  MOD_RES 11: note = Serine at position 11 has a protected side chain (-OtBu)
  MOD_RES 12: note = Serine at position 12 has a protected side chain (-OtBu)
  MOD_RES 18: note = Serine at position 18 has a protected side chain (-OtBu)
  MOD_RES 18: note = Serine at position 18 is amidated with a hydrophilic linker (Rink-(AEEA)6-NH2)
FVQWLIAGGPSSGAPPPS

SEQ ID NO: 22 - protein, 25 aa, synthetic construct
  MOD_RES 1: note = Aspartic acid at position 1 has a protected side chain (-CO2tBu)
  MOD_RES 2: note = Lysine at position 2 has a protected side chain (-NH-Boc)
  MOD_RES 5: note = Glutamine at position 5 has a protected side chain (-CONH-trityl)
  MOD_RES 6: note = Lysine at position 6 is chemically modified by conjugation of the epsilon-amino group of the Lys side chain with (AEEA)2-gamma glutamic acid(OtBu)-CO-(CH2)18-CO2tBu
  MOD_RES 10: note = Glutamine at position 10 has a protected side chain (-CONH-trityl)
  MOD_RES 11: note = Tryptophan at position 11 has a protected side chain (-NBoc-)
  MOD_RES 18: note = Serine at position 18 has a protected side chain (-OtBu)
  MOD_RES 19: note = Serine at position 19 has a protected side chain (-OtBu)
  MOD_RES 25: note = Serine at position 25 has a protected side chain (-OtBu)
  MOD_RES 25: note = Serine at position 25 is amidated with a hydrophilic linker (Rink-(AEEA)6-NH2)
DKIAQKAFVQWLIAGGPSSGAPPPS

SEQ ID NO: 23 - protein, 32 aa, synthetic construct
  MOD_RES 1: note = Serine at position 1 has a protected side chain (-OtBu)
  MOD_RES 2: note = Aspartic acid at position 2 has a protected side chain (-CO2tBu)
  MOD_RES 3: note = Tyrosine at position 3 has a protected side chain (-OtBu)
  MOD_RES 4: note = Serine at position 4 has a protected side chain (-OtBu)
  NON_STD 6: note = X at position 6 is nonnaturally occurring amino acid 2-Aminoisobutyric Acid (Aib)
  MOD_RES 8: note = Aspartic acid at position 8 has a protected side chain (-CO2tBu)
  MOD_RES 9: note = Lysine at position 9 has a protected side (-NHBoc)
  MOD_RES 12: note = Glutamine at position 12 has a protected side chain (-CONH-trityl)
  MOD_RES 13: note = Lysine at position 13 is chemically modified through conjugation to the epsilon-amino group of the Lys side-chain with -(AEEA)2-gamma glutamic acid(OtBu)-CO-(CH2)18-CO2tBu
  MOD_RES 17: note = Glutamine at position 17 has a protected side chain (-CONH-trityl)
  MOD_RES 18: note = Tryptophan at position 18 has a protected side chain (-NBoc-)
  MOD_RES 25: note = Serine at position 25 has a protected side chain (-OtBu)
  MOD_RES 26: note = Serine at position 26 has a protected side chain (-OtBu)
  MOD_RES 32: note = Serine at position 32 has a protected side chain (-OtBu)
  MOD_RES 32: note = Serine at position 32 is amidated with a hydrophilic linker (Rink-(AEEA)10-NH2)
SDYSIXLDKIAQKAFVQWLIAGGPSSGAPPPS

SEQ ID NO: 24 - protein, 4 aa, synthetic construct
  MOD_RES 1: note = Proline at position 1 is -Fmoc protected
  MOD_RES 4: note = Serine at position 4 has a protected side chain (-OtBu)
PPPS

SEQ ID NO: 25 - protein, 4 aa, synthetic construct
  MOD_RES 1: note = Serine at position 1 is -Fmoc protected
  MOD_RES 1: note = Serine at position 1 has a protected side chain (-OtBu)
  MOD_RES 2: note = Serine at position 2 has a protected side chain (-OtBu)
SSGA

SEQ ID NO: 26 - protein, 4 aa, synthetic construct
  MOD_RES 1: note = Alanine at position 1 is -Fmoc protected
AAGP

SEQ ID NO: 27 - protein, 4 aa, synthetic construct
  MOD_RES 1: note = Glutamine at position 1 is -Fmoc protected
  MOD_RES 1: note = Glutamine at position 1 has a protected side chain (-CONH-trityl)
  MOD_RES 2: note = Tryptophan at position 2 has a protected side chain (-NBoc-)
QWLI

SEQ ID NO: 28 - protein, 4 aa, synthetic construct
  MOD_RES 1: note = Lysine at position 1 is -Fmoc protected
  MOD_RES 1: note = Lysine at position 1 is chemically modified by conjugation of the epsilon-amino group of the Lys side chain with (AEEA)2-gamma glutamic acid(OtBu)-CO-(CH2)18-CO2tBu
KAFV

SEQ ID NO: 29 - protein, 4 aa, synthetic construct
  MOD_RES 1: note = Lysine at position 1 is -Fmoc protected
  MOD_RES 4: note = Glutamine at position 4 has a protected side chain (-CONH-trityl)
KIAQ

SEQ ID NO: 30 - protein, 4 aa, synthetic construct
  MOD_RES 1: note = Isoleucine at position 1 is -Fmoc protected
  MOD_RES 2: note = Alanine at position 2 is alpha-methylated (Aib)
  MOD_RES 4: note = Aspartic acid at position 4 has a protected side chain (-CO2tBu)
IALD

SEQ ID NO: 31 - protein, 4 aa, synthetic construct
  MOD_RES 1: note = Serine at position 1 is -Fmoc protected
  MOD_RES 1: note = Serine at position 1 has a protected side chain (-OtBu)
  MOD_RES 2: note = Aspartic acid at position 2 has a protected side chain (-CO2tBu)
  MOD_RES 3: note = Tyrosine at position 3 has a protected side chain (-OtBu)
  MOD_RES 4: note = Serine at position 4 has a protected side chain (-OtBu)
SDYS

SEQ ID NO: 32 - protein, 39 aa, synthetic construct
  NON_STD 2: note = X at position 2 is nonnaturally occurring amino acid 2-Aminoisobutyric Acid (Aib)
  NON_STD 13: note = X at position 13 is nonnaturally occurring amino acid 2-Aminoisobutyric Acid (Aib)
  MOD_RES 20: note = Lysine at position 20 is is chemically modified through conjugation to the epsilon-amino group of the Lys side-chain with (AEEA)2-gamma glutamic acid-CO-(CH2)18-CO2H
  MOD_RES 39: note = Serine at position 39 is amidated as a C-terminal primary amide
YXEGTFTSDYSIXLDKIAQKAFVQWLIAGGPSSGAPPPS

(19) United States
(12) Patent Application Publication
(10) Pub. No.: US 2025/0188019 A1
(43) Pub. Date: Jun. 12, 2025

(54) COMPOUNDS AND METHODS FOR LIQUID PHASE SYNTHESIS

(71) Applicant: Eli Lilly and Company, Indianapolis, IN (US)

(72) Inventors: Michael Eugene KOPACH, Greenwood, IN (US); John LEE, Carmel, IN (US); Emily Suzanne MURZINSKI, Avon, IN (US); Vineeta RUSTAGI, Indianapolis, IN (US); Heba Azmy Fahim Ibrahim SALIM, Indianapolis, IN (US)

(21) Appl. No.: 18/683,933

(22) PCT Filed: Aug. 23, 2022
(86) PCT No.: PCT/US2022/075307

§ 371 Date: Feb. 15, 2024

Related U.S. Application Data
(60) Provisional application No. 63/260,496, filed on Aug. 23, 2021.

(51) Int. Cl. C07C 233/51; C07C 237/52; C07K 5/103; C07K 7/06; C07K 14/00

(52) U.S. Cl. CPC C07C 233/51; C07C 237/52; C07K 5/1008; C07K 7/06; C07K 14/001; C07C 2603/32

(57) ABSTRACT

The present disclosure comprises hydrophilic linker compounds of Formula 1:

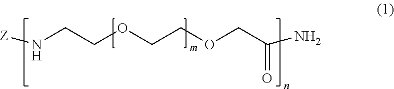

and describes methods for their use in liquid phase synthesis. In Formula 1, “m” is 0 to 20, “n” is 1 to 50, and “Z” is a linker compound as described herein.

The present application is being filed along with a Sequence Listing in ST.26 XML format. The Sequence Listing is provided as a file titled “X22492 Sequence Listing” created 22 Aug. 2022 and is 92 kilobytes in size. The Sequence Listing information in the ST.26 XML format is incorporated herein by reference in its entirety.

### BACKGROUND

Chemical synthesis of polypeptides and amino acid sequences is accomplished by an iterative process in which amino acids are coupled to each other in sequence. The process involves a repeating process of deprotection of either the C- or N-terminus of the growing peptide chain, coupling a protected amino acid thereto, and then deprotecting the newly coupled amino acid, which prepares it for the next coupling in the sequence. This iterative process, in which amino acid coupling and deprotection steps are repeated over and over, amplifies the major challenge in chemical synthesis-namely that of separating the desired synthetic product from other reaction components (solvents, unreacted starting materials and reagents, and undesired reaction by-products).

Solid Phase Peptide Synthesis (“SPPS”) is the method and system that is most commonly used to synthesize polypeptides and amino acid sequences. SPPS involves coupling an activated amino acid to a solid support. This solid support is usually a polymeric resin bead that is functionalized (such as with an NH₂ group). The next amino acid (which generally has its NH₂ terminus protected via a Fmoc, BOC or other protecting group) is reacted with the resin such that the functionalized group on the resin reacts with and binds to the activated COOH group of the terminal amino acid. In this manner, the terminal amino acid is covalently attached to the resin.

Then, in the next step, the NH₂ terminus of the terminal amino acid is de-protected, thereby exposing its NH₂ group for the next reaction. Accordingly, a new amino acid is introduced. This new amino acid has its NH₂ terminus protected via a protecting group (such as an Fmoc, BOC or another protecting group). As such, when this new amino acid is added, the activated ester from the new amino acid reacts with the newly de-protected NH₂ group of the terminal amino acid, thereby coupling these two amino acids together. Once this new amino acid has been coupled, it likewise has a protected NH₂ group that may be subsequently de-protected and reacted with the next amino acid. By doing this repetitive, iterative process over and over, the entire amino acid sequence may be constructed. Once the entire sequence has been constructed, the sequence may be uncoupled (cleaved) from the resin and deprotected, thereby producing the amino acid sequence. (It should be noted that the side chains of the various amino acids (R₁, R₂, etc.) that are added via this process may be orthogonally protected via groups such as BOC, t-butyl or trityl, etc. to prevent such side chains from reacting during the amino acid synthesis process. Those skilled in the art will appreciate how such side chains or other group may be constructed, protected, and subsequently de-protected during the synthesis process.

At each step of the SPPS process, the growing polypeptide remains attached to the solid support, which remains separate from other reaction components by phase separation. The solid support facilitates the separation by enabling separation by filtration of the desired product while attached to the solid support. SPPS is used commercially and is still the standard in peptide synthesis. However, it has a drawback in that it is expensive, time consuming and generates high levels of process waste due to the extensive resin washing which is required. Each amino acid that is added must be de-protected and coupled, which is difficult and usually results in large quantities of solvents being used. Multiple solvent washes are often required after each reaction to remove residual reagents from the resin. The cycle time in a manufacturing facility can be approximately one amino acid coupling and deprotection cycle per day. Making matters worse is that many of these solvents are not environmentally friendly. Also, the phase separation in SPPS presents difficulties in obtaining high product purity. Because the growing polypeptide is not in the same phase as the other reaction components, reaction kinetics are slower than in the liquid phase, and it can be challenging to maximize conversion to desired product while minimizing undesired side reactions such as aggregation. Reaction monitoring and optimization of heterogeneous reaction mixtures can be difficult, particularly when using analytical methods which require analytes to be dissolved in a homogenous liquid stream, such as high-performance liquid chromatography (“HPLC”).

In contrast to SPPS, Liquid Phase Peptide Synthesis (“LPPS”) refers to methods in which polypeptides are prepared in homogenous reaction conditions. This can include synthetic methods involving soluble polymeric support moieties upon which the polypeptide can be prepared in an iterative deprotection and coupling process similar to that used in SPPS. By allowing the deprotection and coupling reactions to occur in a homogenous solution phase, LPPS can overcome some of the difficulties involved in SPPS. In general, LPPS can be more materially efficient than SPPS by requiring less solvent, starting materials, and reagents. Also, liquid-phase reaction kinetics can be faster as compared to reactions which occur at a phase boundary. This can facilitate reaction optimization efforts, for example allowing deprotection steps to occur in less time or under milder reaction conditions to minimize racemization of amino acid residues in the growing polypeptide. LPPS also allows for reaction monitoring directly, for example by HPLC coupled with mass spectrometry (“LCMS”), in which the product attached to a soluble polymeric support can be detected and quantified rather more simply than in an analogous SPPS process.

Despite the advantages in LPPS compared to SPPS, successfully implementing LPPS strategy can be challenging. There remains the problem of isolating the desired product and separating it efficiently from the other reaction components and undesired by-products. To that end, a number of strategies have been employed to enable separation of the polypeptide product from liquid-phase reaction mixtures. Hydrophobic soluble linker systems have been developed as tag-assisted LPPS supports (see e.g., Takahashi, D.; et al. (2017) Angewandte Chemie International Edition 129:7911-7915 and US Patent Application Publication No. US 2018/0215782). Using these linker systems, peptides can be elongated on the linker and then by-products are removed either by precipitation or by extractive aqueous workup. However, it has been found that there are some key limitations such as length of peptide where solubility issues become a major issue as peptide chain elongates. In addition, there can be purity challenges because aqueous washes can have limited efficacy at removing reagents and by-products. These components can interfere in downstream synthetic steps and lead to unfavorable additions and deletions. Furthermore, high residual water in the organic layer can have a negative impact on peptide couplings which may necessitate addition of a de-watering step.

Hydrophilic linker systems can offer critical advantages relative to the hydrophobic linker systems. In these systems, the linker features a hydrophilic “tag,” which enables reaction by-products to be removed by simple extraction with a more environmentally friendly organic solvent. Polyethylene glycol (PEG) has been reported as a hydrophilic support for liquid-phase peptide synthesis (see e.g., Fischer, P. M.; Zheleva, D. I. (2002) Journal of Peptide Science 8:529-542). However, PEG derivatives used in these applications are often polydisperse and lack a fixed molecular weight. Variable PEG chain lengths can create complications in analysis and purification of high molecular weight PEG conjugation products. Accordingly, it would be an improvement to find new hydrophilic linker systems with a fixed molecular weight for LPPS which would address these deficiencies, especially in the commercial manufacturing of peptides. It would be a further advancement if such a system could enable a liquid phase peptide synthetic method for long peptides (15-mer and above). In fact, the present embodiments will specifically provide hydrophilic linker systems for tag-assisted LPPS and methods of use thereof for the synthesis of peptides on a commercial scale. Such methods and systems are disclosed herein.

### SUMMARY

The present embodiments provide compounds of a fixed molecular weight which are useful as hydrophilic linker constructs for liquid phase organic synthesis such as LPPS. Compounds of the present disclosure feature repeating heterobifunctional PEG-like units attached to a linker, upon which a polypeptide or other molecule can be built through coupling (e.g., amino acid coupling) and deprotection steps. In particular, compounds of the present disclosure can be used to build polypeptides or other molecules through repeated synthetic steps, e.g., amino acid coupling and deprotection steps.

An embodiment of the present disclosure comprises hydrophilic linker compounds of Formula 1:

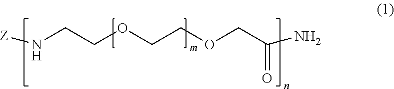

wherein “m” is 0 to 20, “n” is 1 to 50, and “Z” is a linker group. “Z” is a functional group which can form a covalent bond to an optionally protected compound such as an amino acid, which can in turn undergo iterative deprotection and coupling steps onto one or more optionally protected compounds such as amino acids or peptides, and then the resulting product such as a polypeptide product is able to be liberated from the “Z” group through chemical transformation.

Another embodiment of the present disclosure comprises a compound of Formula 1 wherein “m” is 0, 1, 2, or 3 and “n” is 1 to 50. Another embodiment of the present disclosure comprises a compound of Formula 1 wherein “m” is 0, 1, 2, or 3 and “n” is 1 to 10. Another embodiment of the present disclosure comprises a compound of Formula 1 wherein “m” is 1 and “n” is 2 to 10.

A further embodiment of the present disclosure comprises a compound of Formula 1 wherein “Z” is selected from:

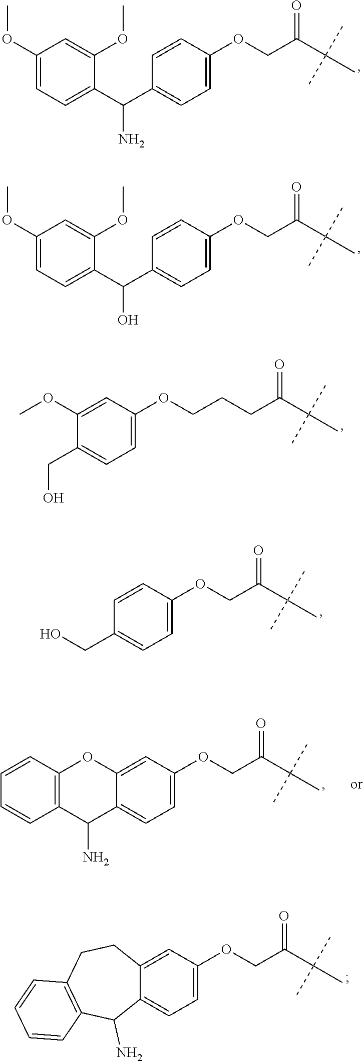

and “m” and “n” are as defined above.

The hydrophilic linkers of the present disclosure are useful for the synthesis of peptides and other compounds, wherein the activated ester of compounds such as an amino acid or peptide fragment is first coupled onto the free alcohol —OH or amine —NH₂ of a linker of Formula 1. Thereafter the compound, e.g., peptide, is grown by coupling activated esters of individual molecule components such as amino acids or peptide fragments consecutively after intermediary deprotections. The finished compounds, e.g., peptides are cleaved from the linker with acidic conditions.

### DETAILED DESCRIPTION

The present embodiments provide hydrophilic linker compounds for use in liquid phase synthesis systems such as LPPS systems that have fixed molecular weights. When used for LPPS, the disclosed compounds enable a liquid phase peptide synthetic method for long peptides (15-mer and above). In fact, the present embodiments will specifically provide hydrophilic linker systems for liquid phase synthesis such as LPPS and methods of use thereof for the synthesis of molecules or peptides on a commercial scale.

As noted above, the present hydrophilic linker compounds may be a compound of Formula 1 outlined above. Specific preferred examples include:
the compound of Formula 1a:

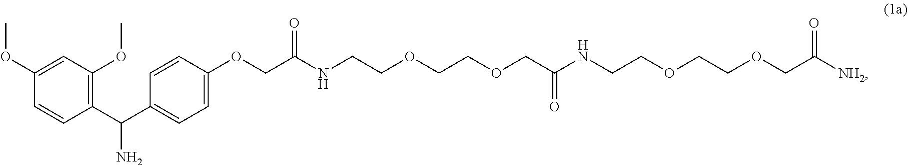 [SMILES: COc1ccc(C(N)c2ccc(OCC(=O)NCCOCCOCC(=O)NCCOCCOCC(N)=O)cc2)c(OC)c1]

the compound of Formula 1b:

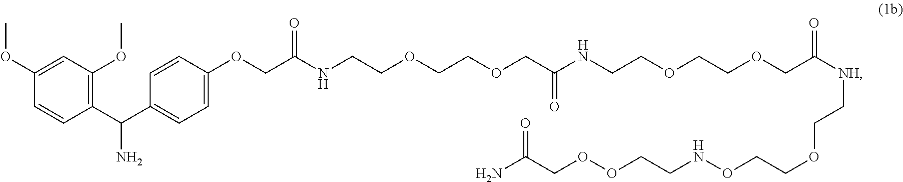 [SMILES: COc1ccc(C(N)c2ccc(OCC(=O)NCCOCCOCC(=O)NCCOCCOCC(=O)NCCOCCONCCOOCC(N)=O)cc2)c(OC)c1]

the compound of Formula 1c:

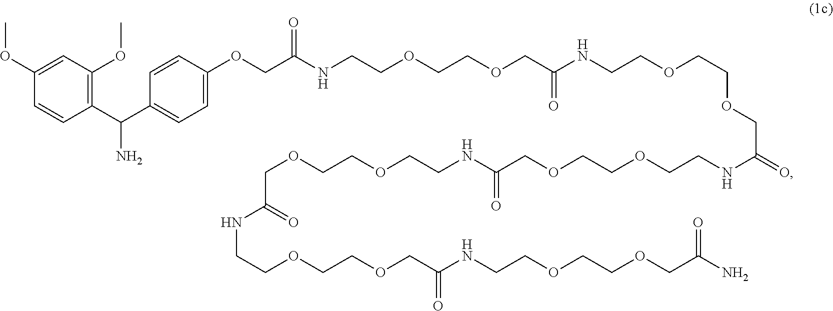 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

the compound of Formula 1d:

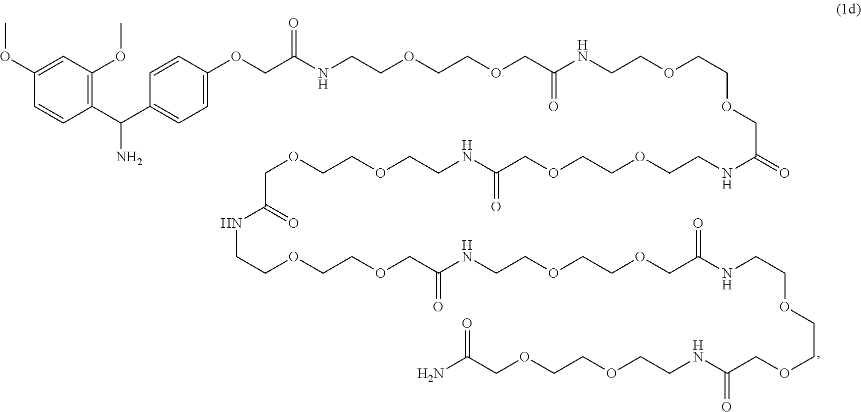 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

the compound of Formula 1e:

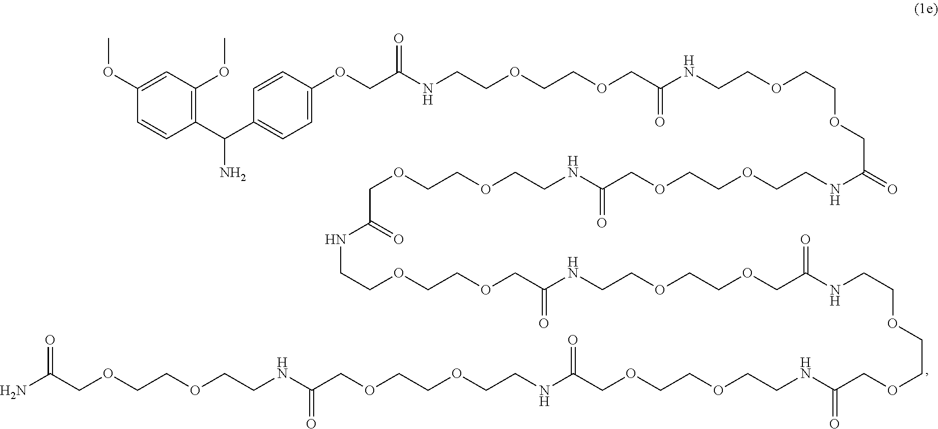 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

the compound of Formula 1f:

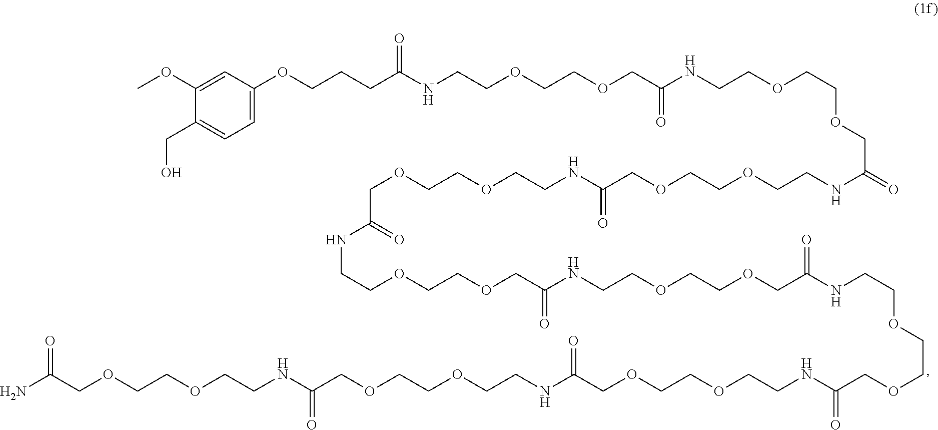 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1CO]

the compound of Formula 1g:

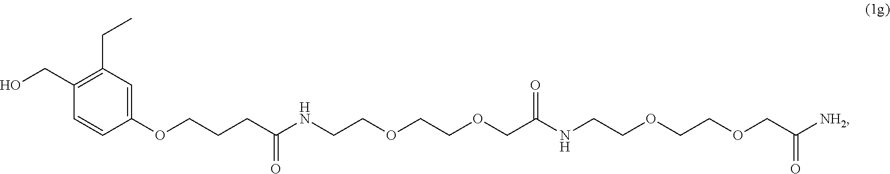 [SMILES: CCc1cc(OCCCC(=O)NCCOCCOCC(=O)NCCOCCOCC(N)=O)ccc1CO]

the compound of Formula 1h:

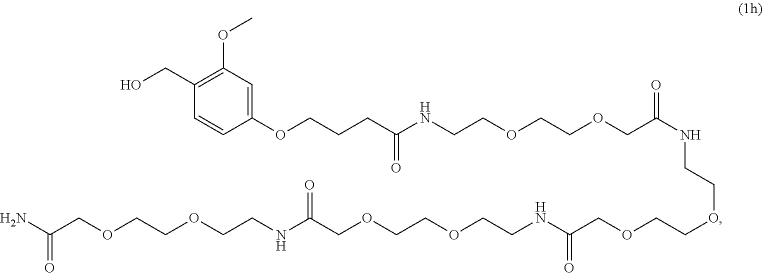 [SMILES: COc1cc(OCCCC(=O)NCCOCCOCC(=O)NCCOCCOCC(=O)NCCOCCOCC(=O)NCCOCCOCC(N)=O)ccc1CO]

the compound of Formula 1i:

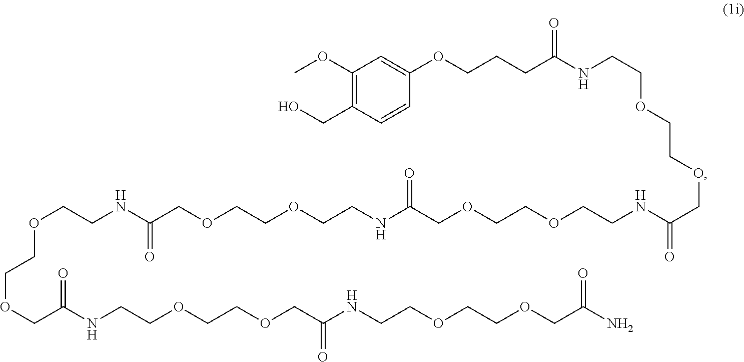 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1CO]

the compound of Formula 1j:

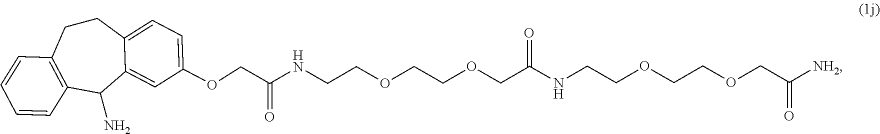 [SMILES: NC(=O)COCCOCCNC(=O)COCCOCCNC(=O)COc1ccc2c(c1)C(N)c1ccccc1CC2]

the compound of Formula 1k:

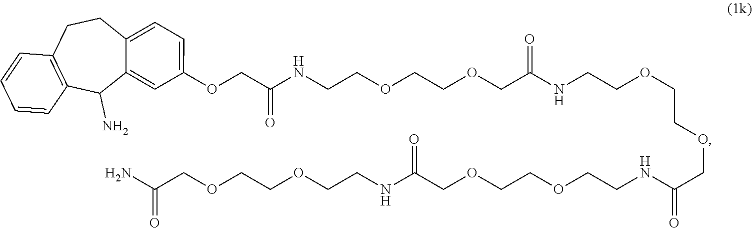 [SMILES: NC(=O)COCCOCCNC(=O)COCCOCCNC(=O)COCCOCCNC(=O)COCCOCCNC(=O)COc1ccc2c(c1)C(N)c1ccccc1CC2]

the compound of Formula 1l:

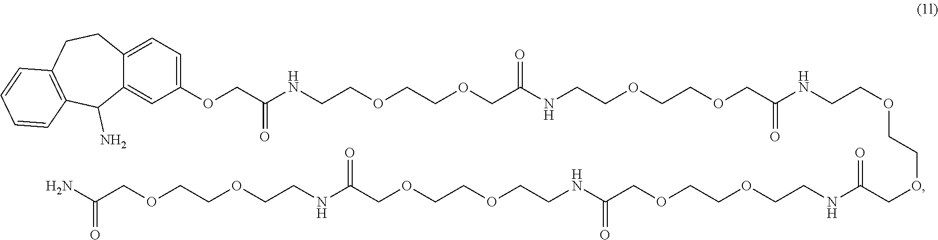 [SMILES: NC(=O)COCCOCCNC(=O)COCCOCCNC(=O)COCCOCCNC(=O)COCCOCCNC(=O)COCCOCCNC(=O)COCCOCCNC(=O)COc1ccc2c(c1)C(N)c1ccccc1CC2]

the compound of Formula 1m:

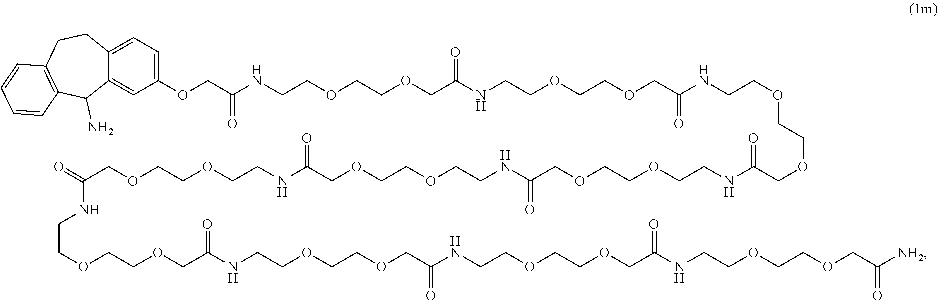 [SMILES: NC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COc1ccc2c(c1)C(N)c1ccccc1CC2]

the compound of Formula 1n:

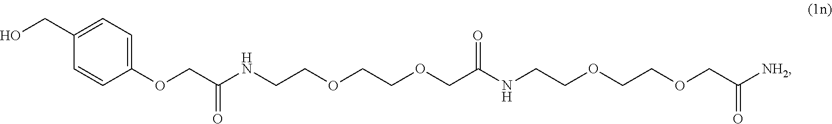 [SMILES: NC(=O)COCCOCCNC(=O)COCCOCCNC(=O)COc1ccc(CO)cc1]

and the compound of Formula 1o:

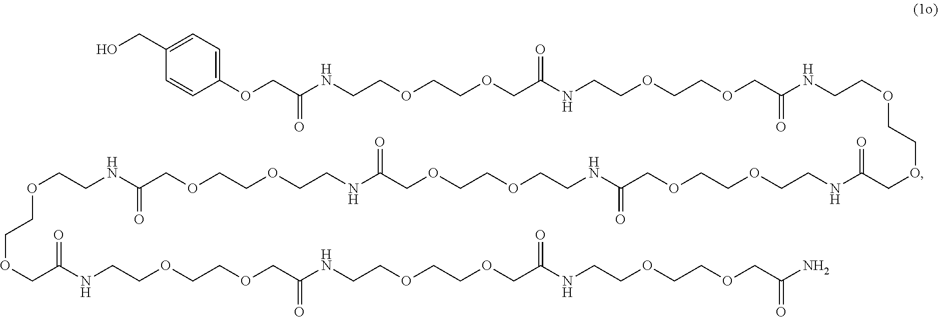 [SMILES: NC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COc1ccc(CO)cc1]

Liquid phase synthesis using the hydrophilic linker compounds described herein can be used to assemble compounds linked through amide bonds, i.e., through the condensation of a carbonyl group on one molecule to the amino group of another molecule. Peptide synthesis is a clear use of the hydrophilic linker molecules and methods described herein as peptide bonds result from the condensation reaction of the carboxyl group of one amino acid to the amino group of another. Thus, compounds containing amide bonds can be assembled through iterations of coupling and deprotection reactions to assemble the molecule, which upon completion must be released from the support used during the synthesis. Specifically, a molecule having an amino group protected by an Fmoc group with an available carboxylic acid group can be coupled onto a hydrophilic linker molecule as described herein. Then the resulting molecule can be deprotected by removing the Fmoc group and the resulting unprotected amino group of the molecule can be coupled to an available carboxylic acid group on a further molecule with an available amino group. Examples 21-24 show the liquid phase synthesis of a non-peptide molecule using hydrophilic linker molecules described herein.

Peptide preparation by both SPPS and LPPS proceeds through iterations of coupling and deprotection reactions to elongate the peptide, which upon completion must be released from the support used during the synthesis. In addition, the amino acid or peptide fragment starting materials used in the synthesis often have side chain protecting groups which help ensure selectivity during coupling steps. The side chain protecting groups are selected so that they are stable to the conditions used during the deprotection steps in the peptide elongation process. For example, FMOC groups can be used to protect the amino group in amino acid starting materials and are easily removed with secondary amine bases. In contrast, BOC and triphenylmethyl (trityl) protecting groups are stable under the basic conditions typically used to remove FMOC groups during peptide elongation, and upon completion can be removed with strong organic acids. Some peptide synthesis linkers can be cleaved under the same conditions used for amino acid side chain deprotections. This is referred to as a “hard” cleavage method—upon completion of the peptide elongation the peptide is simultaneously deprotected and cleaved from the resin. Complex synthetic strategy can be enabled by careful selection of linker chemistry which allows cleavage to occur under conditions orthogonal to those of the side chain deprotections. This is referred to as a “soft” cleavage method—the peptide is cleaved from the resin with some or all of the side chain protecting groups still intact.

The hydrophilic linkers of the present disclosure can be used in synthetic processes which utilize “hard” and “soft” cleavage methods. Using a “soft” cleavage synthetic strategy can, for example, enable the synthesized peptide to be used as a starting material in a hybrid fragment-based synthesis of a more complex peptide. In addition, hybrid SPPS/LPPS processes can be implemented as part of a convergent peptide synthesis strategy using the hydrophilic linkers described herein. Peptide fragments can be built using SPPS, cleaved from their solid support, isolated, and optionally purified, and then assembled by coupling them onto a peptide attached to a hydrophilic linker using LPPS. This convergent hybrid SPPS/LPPS strategy can be more practical and efficient upon scaleup as compared to processes which are entirely SPPS.

A fragment-based convergent peptide synthesis strategy can also be implemented with LPPS using the hydrophilic linkers described herein. In this case, peptide fragments are built using LPPS, cleaved from the hydrophilic linker support, isolated, and optionally purified, and then assembled by coupling them onto a peptide attached to a hydrophilic linker using LPPS.

The hydrophilic linker compounds of the current disclosure can also be used as part of a linker system which facilitates membrane-enhanced peptide synthesis (MEPS). Synthetic strategy built around MEPS employs membrane-based separation (or diafiltration) of the growing peptide from other reaction components. Practical implementation MEPS in a LPPS strategy is facilitated by use of a system that allows this separation to be conducted in the same organic solvent in which the reactions are performed, for example using organic solvent nanofiltration (OSN). Such membrane-based separation techniques achieve separation by the size difference between the growing peptide and the other reaction components. To that end, “nanostar” hub structures can be used as LPPS supports which increase the molecular size of the growing peptide, yet are themselves compact and easily synthesized (see, e.g., Yeo, J.; et al. (2021) Angewandte Chemie International Edition 60:7786-7795). Aromatic hub structures can serve as central attachment points to which peptide synthesis linkers can be attached. These hub structures can also serve as additional UV chromophores useful for reaction monitoring, e.g., by UHPLC-MS (ultra-high performance liquid chromatography-mass spectrometry). Nanostar hubs increase the mass difference between the growing synthetic peptide and other reaction components, increasing diafiltration efficiency.

The hydrophilic linker compounds of the current disclosure can be used as part of a MEPS based strategy. In particular, hydrophilic linker compounds of the current disclosure can be connected to form nanostar hubs. Scheme 1 shows synthesis of previously disclosed nanostar structures featuring polyethylene glycol chains linking either a Rink- or Wang-type linker to a central phenyl ring (Yeo, 2021).

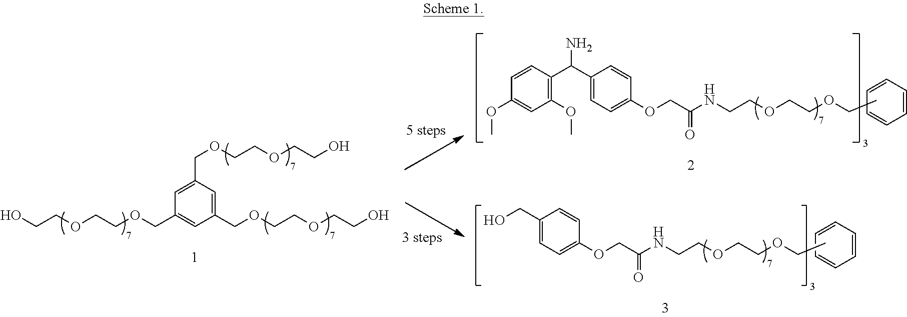

Analogous to nanostar structure 2 in Scheme 1, compounds of Formula 1 described herein could also be attached to a nanostar hub, to give e.g., a compound of Formula 2:

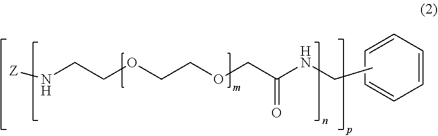

wherein “Z” is

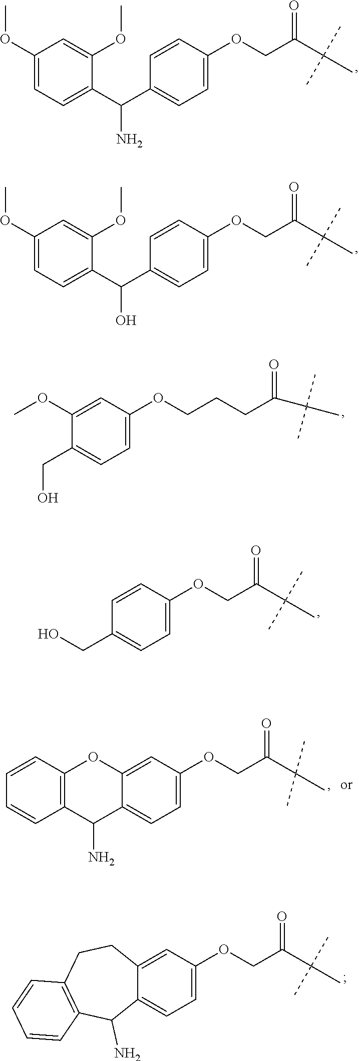

“m” is 0, 1, 2, or 3; “n” is 1 to 10; and “p” is 2 or 3. In particular, nanostar compounds of Formula 2 could be prepared wherein “Z” is

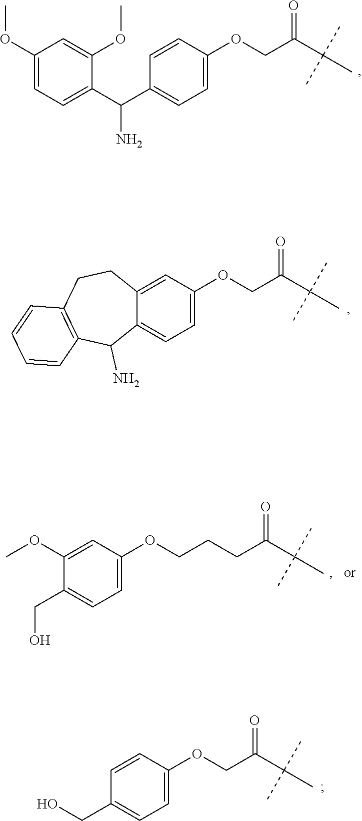

“m” is 1; “n” is 2, 4, 6, 8, or 10; and “p” is 2 or 3.

Also contemplated in the current disclosure are “branched” hydrophilic linker compounds, wherein the linker features two or more hydrophilic functional groups attached to the peptide attachment group. Branched hydrophilic linker systems could comprise compounds of Formula 3:

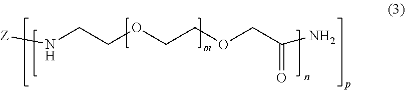

wherein “Z” represents a functional group which can form a covalent bond to an optionally protected amino acid, which can in turn undergo iterative deprotection and coupling steps onto one or more optionally protected amino acids or peptides, and then the resulting polypeptide product is able to be liberated from the “Z” group through chemical transformation; “m” is 0, 1, 2, or 3; “n” is 1 to 10; and “p” is 2 or 3. In particular, branched compounds of Formula 3a, 3b, and 3c could be prepared:

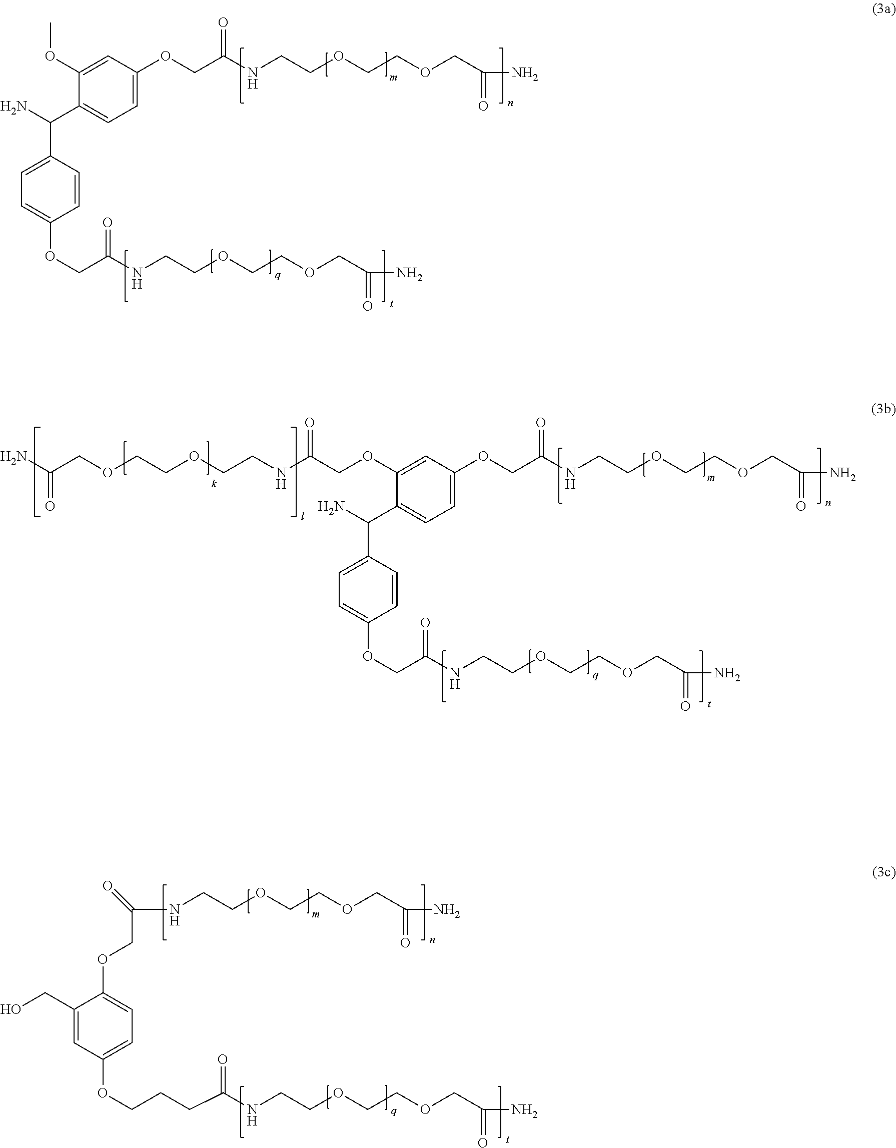

wherein “k” is 1, “m” is 1, “q” is 1; and “l”, “n”, and “t” are each independently 2, 4, 6, 8, or 10.

Significant progress has been made in recent years in technology enabling large scale implementation of flow chemistry processes. In flow chemistry, reagents and reactants are pumped together into a continuously flowing mixture, usually through a tube or pipe. Notable advantages can be realized by implementing a flow chemistry strategy into a chemical manufacturing process when compared to traditional batch processes. Flow chemistry strategy facilitates control over reaction parameters such as pressure, temperature, and reaction time. By passing the reaction mixture through a tube, for example, the mixture is exposed to the tube's high surface area which increases the flux of heat into or out of the reaction, which enables rapid heating or cooling. Flow reactors can be pressurized, allowing heating above the boiling point at atmospheric pressure and increasing reaction rates. Traditional batch processes can become complicated upon scaleup because of mixing and heat transfer rates, whereas flow chemistry processes can more easily maintain a high degree of control over these parameters. Additionally, by performing the reaction in a moving stream, only small amounts of high-energy intermediates are produced at any time during the course of the process, decreasing safety risks associated therewith.

Flow chemistry principles have been applied to both SPPS and liquid phase synthesis systems such as LPPS. In SPPS, packed-bed flow systems have been investigated on both large and small scale, and such systems are highly amenable to automation. In LPPS, immobilized reagents and microreactors have been used to make peptide fragments on small scales (see, e.g Baxendale, I. R.; et al. (2006) Chemical Communications 4835-4837; and Fuse, S.; et al. (2014) Angewandte Chemie International Edition 53:851-855), and continuous stirred-tank reactor (CSTR) technology has been applied to the large-scale preparation of di- and tripeptide products (see e.g., Jolley, K. E.; et al. (2017) Organic Process Research and Development 21:1557-1565).

The hydrophilic linker compounds of the current disclosure are particularly useful in enabling flow chemistry liquid phase processes such as LPPS. Rapid reaction kinetics of coupling and deprotecting reactions on the growing molecule, e.g., a peptide, coupled to the hydrophilic linker in solution is a favorable feature for flow chemistry process implementation. The problem remains in solution-phase flow chemistry of separating desired reaction products from undesired by-products and unreacted starting materials. Preparation of molecules such as peptides using the hydrophilic linker compounds disclosed herein occurs in solution, however the isolation of the desired products occurs at phase separation, allowing the use of continuous liquid-liquid separation (e.g., with mixer-settlers or continuous flow centrifuges).

Significant volume of toxic solvents such as dimethylformamide, N-methyl-2-pyrrolidone, dimethylacetamide, and dichloromethane are utilized in conventional solid phase peptide synthesis, e.g., in washing, coupling and deprotection steps, which poses challenges to industrial hygiene and environmental protection. For this reason, there is a strong interest in developing alternative solvents that are more environmentally friendly (i.e., “greener”) for use in peptide synthesis. The methods described herein can make use of such greener solvents. Examples of greener wash solvents include ethyl acetate, isopropyl acetate, MTBE (methyl tert-butyl ether) and CPME (cyclopentyl methyl ether. The coupling reactions described herein could also occur in greener solvents such as DMSO.

As used herein, the term “amino acid” refers to an organic compound comprising a carboxylic acid (—CO₂H) and an amine (—NH₂) functional group. Amino acids can be proteinogenic (i.e., incorporated biosynthetically into proteins during translation), such as glycine, L-alanine, and L-phenylalanine, or non-proteinogenic such as 3-aminoisobutyric acid and 8-amino-3,6-dioxaoctanoic acid.

As used herein, the term “hydrophilic linker” refers to a chemical moiety upon which molecules such as polypeptides can be built through coupling (e.g., amino acid coupling) and deprotection steps, and which features one or more functional groups which has a high affinity for water.

As used herein, the term “flow chemistry” refers to performing chemical reactions in a continuously flowing stream.

As used herein, the term “nanostar” refers to a linker construction concept used for the synthesis of biopolymers (e.g., polypeptides) wherein a core organic chemical structure serves as a central attachment point (or “hub”) for two or more linkers, upon which biopolymeric chains can be built. Nanostar structures for construction of polypeptides have been described (see e.g., Yeo, J.; et al. (2021) Angewandte Chemie International Edition 60:7786-7795).

As used herein, the term “peptide” or “polypeptide” refers to a polymeric chain of amino acids. These amino acids can be natural or synthetic amino acids, including modified amino acids. As used herein, the terms “peptide” and “polypeptide” are used interchangeably.

Certain abbreviations used herein are defined as follows: “AEEA” refers to 2-(2-(2-aminoethoxy)ethoxy) acetyl; “Aib” refers to 2-aminoisobutyric acid; “Boc” refers to tert-butoxycarbonyl; “CAD” refers to charged aerosol detector; “DCM” refers to dichloromethane; “DEPBT” refers to 3-(diethoxyphosphoryloxy)-1,2,3-benzotriazin-4(3H)-one; “DIC” refers to diisopropylcarbodiimide; “DIEA” refers to diisopropylethylamine; “DMF” refers to N,N-dimethylformamide; “DMSO” refers to dimethylsulfoxide; “DVB” refers to divinylbenzene; “EDC” refers to 1-ethyl-3-(3-dimethylaminopropyl) carbodiimide; “ESMS” refers to electrospray mass spectrometry; “Fmoc” refers to fluorenylmethyloxycarbonyl; “Fmoc-Suberol” refers to 5-Fmoc-amino-2-carboxymethoxy-10,11-dihydro-5H-dibenzo[a,d]cycloheptene; “HMPA” refers to 4-(hydoxymethyl)phenoxyacetic acid; “HMPB” refers to 4-(4-hydroxymethyl-3-methoxyphenoxy) butyric acid; “LCMS” refers to liquid chromatography-mass spectrometry; “LPPS” refers to liquid phase peptide synthesis; “MTBE” refers to methyl tert-butyl ether; “oxyma” refers to ethyl cyanohydroxyiminoacetate; “PEG” refers to polyethylene glycol; “PyBop” refers to (benzotriazol-1-yloxy)tripyrrolidinophosphonium hexafluorophosphate 17; “PyOxim” refers to [(E)-(1-cyano-2-ethoxy-2-oxoethylidene)amino]oxy-tripyrrolidin-1-ylphosphanium; hexafluorophosphate; “SPPS” refers to solid-phase peptide synthesis; “tBu” refers to tert-butyl; “TFA” refers to trifuoroacetic acid; “Trt” refers to trityl; and “UPLC” refers to ultra-performance liquid chromatography.

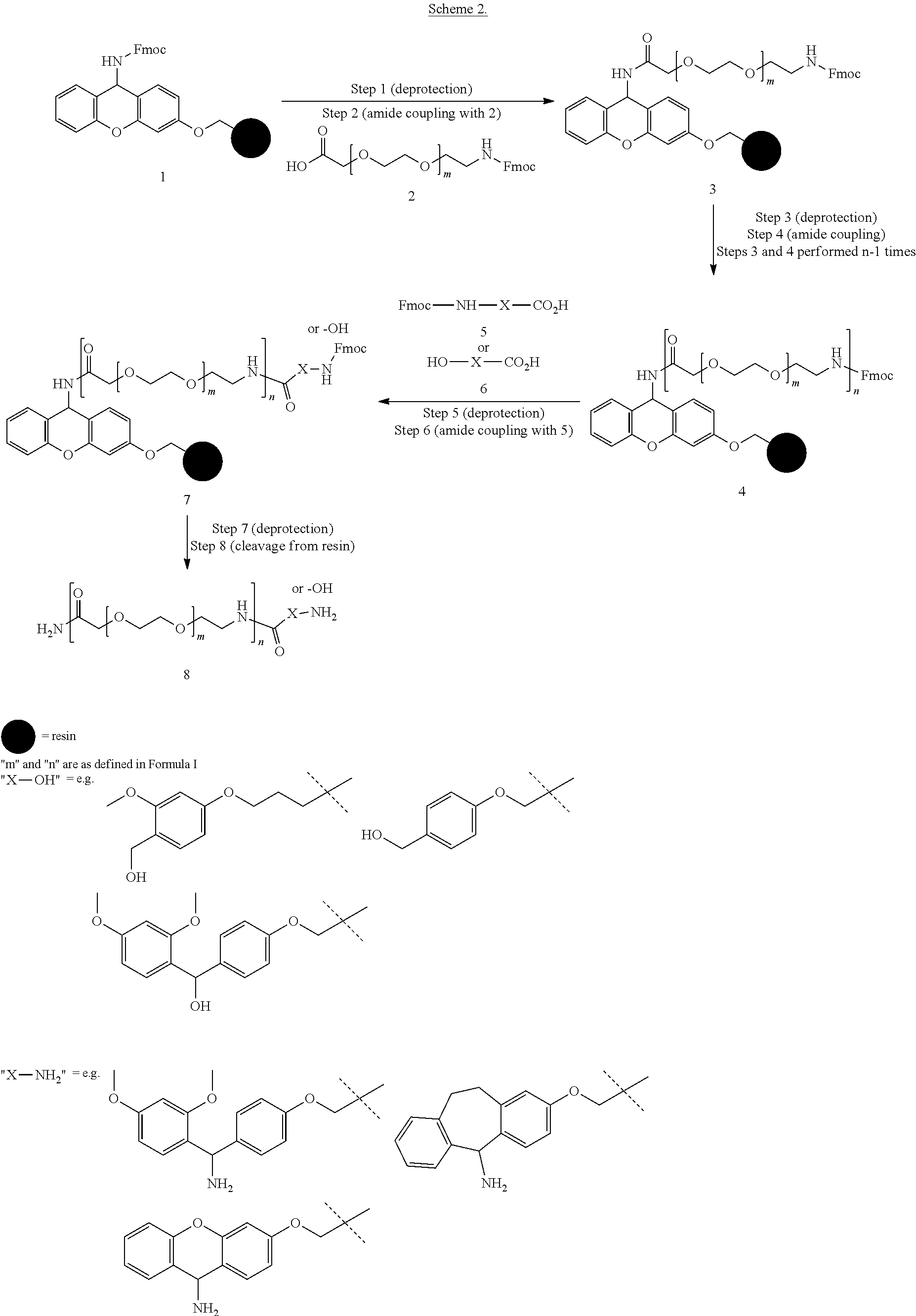

Scheme 2 shows the preparation of hydrophilic linker compound 8, wherein “X” represents a functional group which bears a chemically labile —OH or —NH₂, which can form an ester or amide bond (respectively) to an optionally protected amino acid, which can in turn undergo iterative deprotection and coupling steps onto one or more optionally protected amino acids or peptides, and then the resulting polypeptide product is able to be liberated from the “X” group through chemical transformation.

Compound 8 is prepared in Scheme 1 by solid-phase synthesis using an Fmoc protecting group strategy. This synthesis can be carried out in part or in whole on an automated peptide synthesizer. In Step 1, Fmoc-Sieber amide resin (1) is deprotected with piperidine and then coupled in Step 2 with Fmoc-protected intermediate 2 using amide coupling conditions (e.g., Oxyma and DIC) to give intermediate 3. Iterative cycling of Step 3 (deprotection using piperidine) and Step 4 (amide coupling with Fmoc-protected intermediate 2 using amide coupling conditions e.g., PyOxim and an organic base) gives intermediate 4, the cycle repeated “n” minus one times to achieve “n” monomeric units coupled together. In Step 5, intermediate 4 is deprotected with piperidine and then undergoes amide coupling (e.g., with PyOxim and an organic base) with either intermediate 5 or 6 in Step 6 to give intermediate 7. If intermediate 7 bears an Fmoc-protected nitrogen, it is deprotected in Step 7 using piperidine. Finally, the hydrophilic linker compound 8 is cleaved from the Sieber resin under acidic conditions (e.g., using TFA).

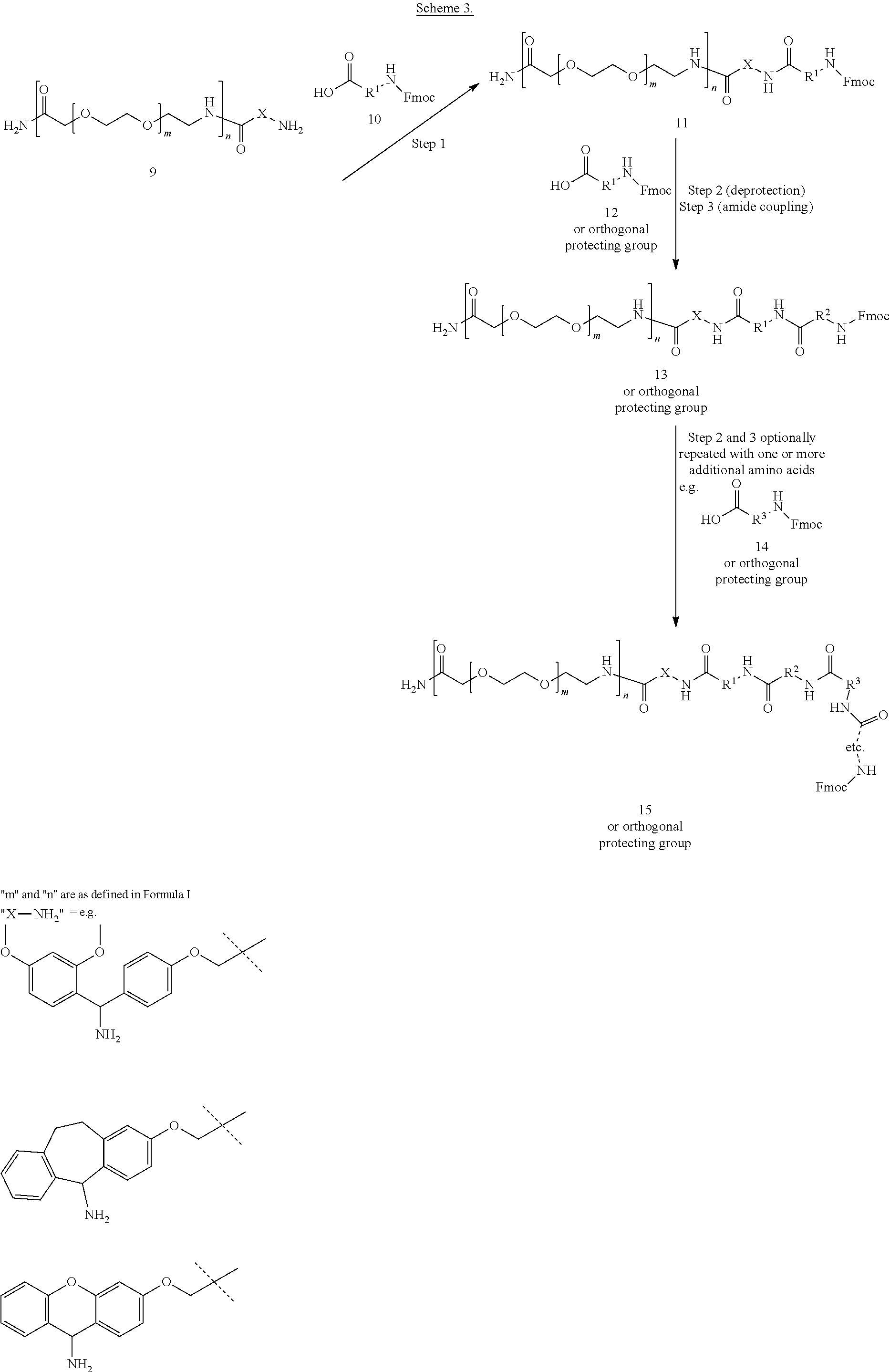

Scheme 3 shows the elongation of polymeric chains of amino acids using hydrophilic linker compound 9 which bears a nitrogen upon which the polymeric chain of amino acids can be built and then cleaved from the linker under acidic conditions. In Step 1, Fmoc-protected amino acid 10 is coupled with hydrophilic linker compound 9 using amide coupling conditions (e.g., PyOxim and an organic base) in a polar aprotic organic solvent such as DMF or DMSO to give the first coupled intermediate 11. Upon completion of the reaction, a less polar aprotic solvent such as MTBE is added, which results in the coupled intermediate 11 to precipitate from the reaction mixture. The precipitate is separated from the bulk reaction mixture (e.g., by centrifugation and decanting the supernatant) and optionally washed by treating it again with a solvent in which it is insoluble (e.g., MTBE) followed by separation of the precipitate (e.g., by centrifugation and decanting the supernatant). In this manner intermediate 11 is isolated from the reaction mixture and separated from the bulk of reaction solvents, unreacted starting materials, and reaction waste products. In Step 2, intermediate 11 is deprotected using piperidine and the precipitation/product separation/optional washing procedure is performed, then in Step 3 the next protected amino acid (12) is coupled using amide coupling conditions (e.g., PyOxim and organic base) followed by the precipitation/product separation/optional washing procedure to give intermediate 13. At this point, the intermediate 13 can be carried on to other chemical transformations (e.g., as outlined in Scheme 6). If the terminal nitrogen protecting group is-Fmoc and polymeric chain elongation is to continue, Steps 2 and 3 are repeated iteratively with protected amino acids (e.g., 14) in sequence to give intermediate 15.

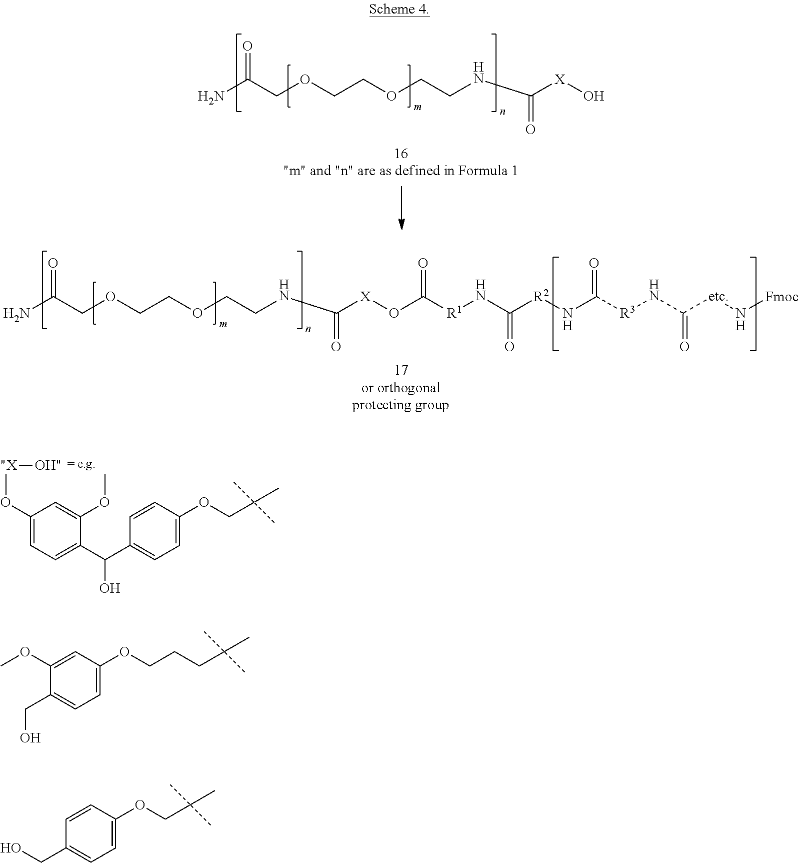

Scheme 4 shows the elongation of polymeric chains of amino acids using hydrophilic linker compound 16 which bears an oxygen upon which the polymeric chain of amino acids can be built and then cleaved from the linker under acidic conditions. The steps of this process are analogous to the steps outlined in Scheme 3, except that Step 1 is an esterification step (carried out using reagents e.g., PyBop/organic base or DIC/DMAP). Iterative deprotection and coupling steps with protected amino acids as outlined in Scheme 3 (Steps 2 and 3, respectively) give the polymeric compound 17.

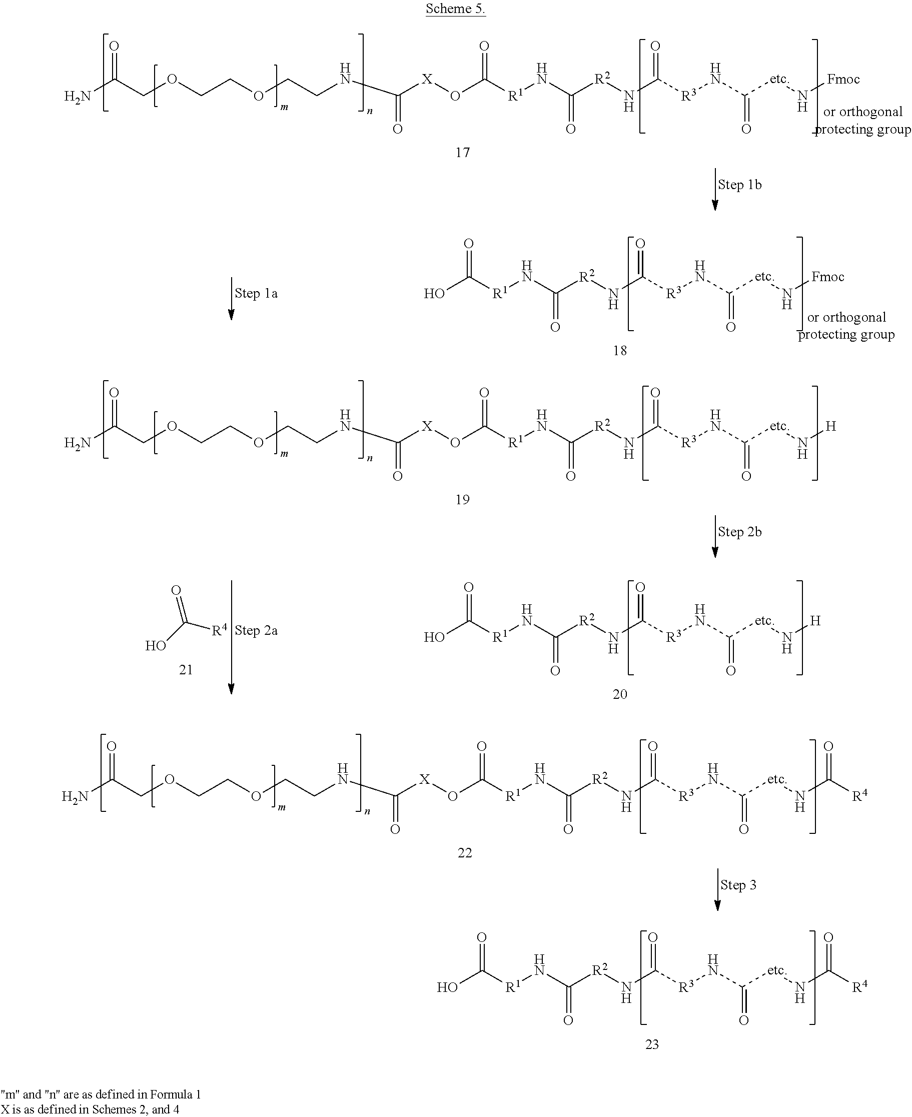

Scheme 5 shows three pathways for cleaving the elongated amino acid polymer off of linkers connected by an oxygen. When cleaved from these linkers, the amino acid polymer has a free carboxylic acid (—CO₂H) at its C-terminus.

In the first pathway, in Step 1b compound 17 undergoes “soft” cleavage under acidic conditions (e.g., 2-5% TFA in DCM), hydrolyzing the linker from the amino acid polymer to give a carboxylic acid group at the C-terminus and leaving the N-terminal protecting group (and other protecting groups which may be present in R¹, R², R³, etc.) intact in compound 18. Neutralization with a base such as pyridine followed by aqueous workup separates the bulk of the hydrophilic linker from 18. With the protecting group at the N-terminus intact, 18 can then be coupled with another amine, e.g., as part of a fragment-based peptide synthetic strategy.

In the second pathway, in Step 1a, the N-terminal protecting group is removed under suitable conditions (in the case of -Fmoc protection, piperidine is used) to give 19. In Step 2b The amino acid polymer can be hydrolyzed from the linker to give 20 under conditions which leave protecting groups which may be present in R¹, R², R³, etc. intact (e.g., using 2-5% TFA in DCM), or a global deprotection of acid-labile protecting groups can be achieved under “hard” cleavage conditions using e.g., a mixture of TFA, triisopropylsilane, 1,2-ethanedithiol, and water (85:5:5:5 v/v ratio).

In the third pathway, intermediate 19 is coupled with carboxylic acid 21 in Step 2a under amide coupling conditions (e.g., DEPBT and an organic base, or 21 can be reacted as a succinimidyl ester using an organic base) to give 22. In Step 3, compound 23 is cleaved from the hydrophilic linker using either the “hard” or “soft” cleavage outlined above.

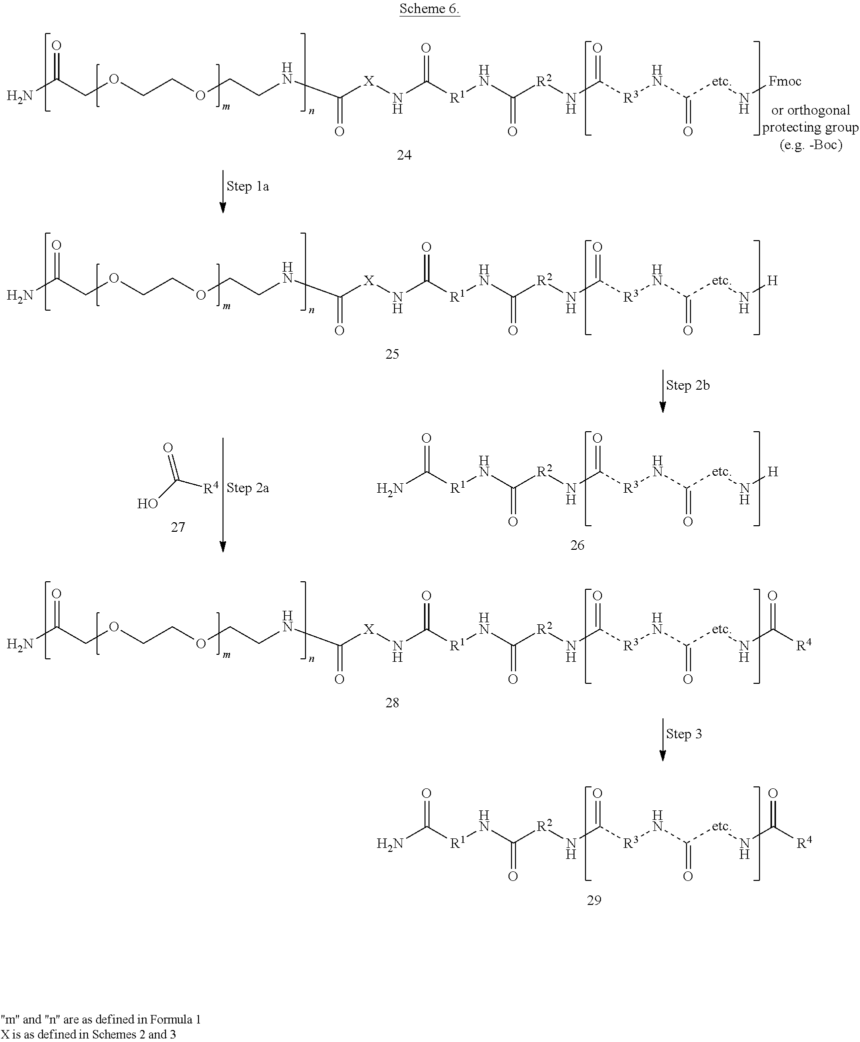

Scheme 6 shows two pathways for cleaving the elongated amino acid polymer off of linkers connected by a nitrogen. When cleaved from these linkers, the amino acid polymer has a primary amide (—CONH₂) at its C-terminus.

In the first pathway, the N-terminal protecting group on intermediate 24 is removed (using piperidine if the protecting group is-Fmoc) in Step 1a to give 25 and then cleavage from the hydrophilic linker is achieved under acidic conditions, either using “soft” cleavage conditions (e.g., 2-5% TFA in DCM) leaving R¹, R², R³, etc. protecting groups intact, or using “hard” cleavage conditions (e.g., 85:5:5:5 TFA, triisopropylsilane, 1,2-ethanedithiol, and water) to remove acid labile R₁, R₂, R³, etc. protecting groups to give 26. If the N-terminal protecting group of intermediate 24 is an acid-labile protecting group such as -Boc, Steps 1a and 2b can be achieved in one pot under “hard” cleavage conditions.

In the second pathway, 25 is coupled with carboxylic acid 27 in Step 2a under amide coupling conditions (e.g., DEPBT and an organic base, or 27 can be reacted as a succinimidyl ester using an organic base) to give 28. In Step 3, compound 29 is cleaved from the hydrophilic linker using either the “hard” or “soft” cleavage outlined above.

### EXAMPLES

The following Examples further illustrate various embodiments of the disclosure and represent typical synthesis of the compounds of the disclosure. The reagents and starting materials are readily available or may be readily synthesized by one of ordinary skill in the art. It should be understood that the Examples are set forth by way of illustration and not limitation, and that various modifications may be made by one of ordinary skill in the art.

LCMS was performed on an AGILENT® HP1200 liquid chromatography system. Chromatography conditions—column: Waters CSH™ C₁₈ 150×2.1 mm, 1.7 μm; gradients used were 5 to 95% solvent B: solvent A over a 20 to 30 min run; flow rate: 0.5 mL/min; column temperature: 40 to 50° C.; Solvent A: 0.2% TFA in water; Solvent B: acetonitrile. Electrospray mass spectrometry measurements (ESMS) were performed on a Mass Selective Detector quadrupole mass spectrometer interfaced to the chromatography system.

### Example 1

### Preparation of 2-(4-(amino(2,4-dimethoxyphenyl)methyl)phenoxy)-N-(53-amino-8,17,26,35,44,53-hexaoxo-3,6,12,15,21,24,30,33,39,42,48,51-dodecaoxa-9,18,27,36,45-pentaazatripentacontyl)acetamide[Rink-(AEEA)₆-NH₂]

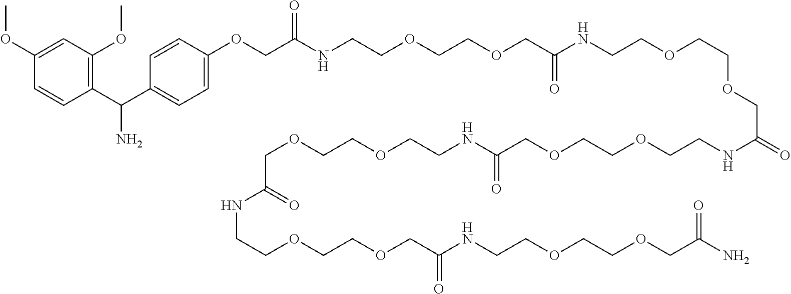 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

The title compound was prepared by solid-phase synthesis using Fmoc strategy on a Symphony X Automated Peptide Synthesizer (Protein Technologies Inc.) starting from Fmoc-Sieber amide resin (substitution 0.8, styrene 1% DVB, 100-200 mesh).

Coupling AEEA units on Sieber resin: The preparation of (AEEA)₆ on Fmoc-Sieber amide resin was performed in 9 batches on a 0.5 mmol scale (4.5 mmol total). For each batch, the resin was swelled using two washes of DMF (10 mL) over 10 min each. Then, deprotections and coupling cycles were performed as follows: washed the resin with DMF (9 mL over 2 min), deprotected using 20% piperidine in DMF (7 mL over 5 min then 9 mL over 25 min), washed with DMF (9 mL over 1 min, repeated 6 times), coupled with Fmoc-AEEA-OH [0.375 M Fmoc-AEEA-OH in DMF (4 mL, 1.5 mmol, 3 equiv), Oxyma (0.750 M, 2 mL, 3 equiv), DIC (0.660 M, 2.5 mL, 3.3 equiv)] mixed by nitrogen bubbling for 1 h 45 min, and finally the reaction vessel was drained and washed with DMF (9 mL over 30 sec, repeated 3 times). This deprotection and coupling cycle was repeated a total of six times to give Fmoc-(AEEA)₆ on Sieber resin. After the final DMF wash, the resin was washed with DCM (10 mL over 1 min, repeated 5 times) and dried under a flow of nitrogen for 4 h. The average yield of Fmoc-(AEEA)₆ on Sieber resin for each batch prepared this way was 1.156 g.

Coupling Rink group onto (AEEA)₆ on Sieber resin: A portion of Fmoc-(AEEA)₆ on Sieber resin (1.1 g, 0.5 mmol) was swelled with DMF (10 mL over 20 min, repeated 3 times), deprotected using 20% piperidine in DMF (10 mL over 20 min, repeated three times), then washed with DMF (10 mL over 2 min, repeated 5 times). A solution of Fmoc-Rink linker (p-[a-[1-(9H-fluoren-9-yl)-methoxyformamido]-2,4-dimethoxybenzyl]-phenoxyacetic acid, 0.81 g, 1.5 mmol, 3.0 equiv), PyOxim (0.79 g, 1.5 mmol, 3.0 equiv), and DIEA (0.52 mL, 0.39 mg, 3.0 mmol, 6.0 equiv) in DMF (9 mL) was added to the reaction vessel and mixed by nitrogen bubbling for 2 h. The reaction vessel was drained, washed with DMF (10 mL over 2 min, repeated 5 times), then deprotected using 20% piperidine in DMF (10 mL over 20 min, repeated 3 times). The resin was washed with DMF (10 mL over 2 min, repeated 5 times). After the final DMF wash, the resin was washed with DCM (10 mL over 2 min, repeated 5 times) and dried under a flow of nitrogen for 4 h to give 1.21 g Rink-(AEEA)₆ on Sieber resin.

Cleavage of Rink-(AEEA)₆-NH₂ from Sieber resin: Rink-(AEEA)₆ on Sieber resin (1.21 g) was mixed with 5% TFA in DCM (12 mL) for 30 min, filtered, and washed with additional DCM. The filtrate was neutralized with DIEA and concentrated under reduced pressure. The resultant oil was taken up in DMSO (2 mL) and MTBE (30 mL) was added, then the mixture was centrifuged at 3000 rpm for 3 min. The supernatant was decanted, and fresh MTBE (30 mL) was added, then the mixture was centrifuged again at 3000 rpm for 3 min. The supernatant was again decanted, leaving the title compound as the oil sediment. ESMS m/z 1188.5 (M+H⁺).

### Example 2

### Preparation of 2-[2-[2-[[2-[2-[2-[[2-[2-[2-[[2-[2-[2-[[2-[2-[2-[[2-[2-[2- [[2-[2-[2-[[2-[2-[2-[[2-[4-[amino-(2,4-dimethoxyphenyl)methyl]phenoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetamide[Rink-(AEEA)₈-NH₂]

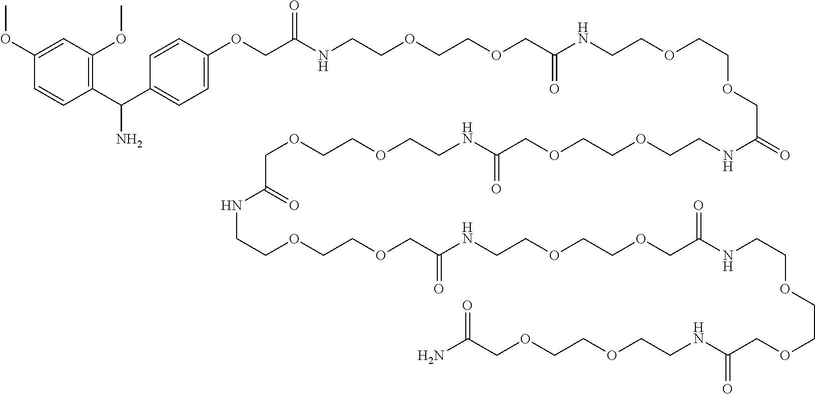 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

The title compound was prepared by solid-phase synthesis using the procedure essentially as described in Example 1, coupling 8 AEEA units onto the solid support before coupling on the Fmoc-Rink linker. After coupling the Fmoc-Rink linker onto the (AEEA)₈ on Sieber resin, the Fmoc-Rink-(AEEA)₈-NH₂ was cleaved from the resin with 2% TFA in DCM (5 volumes over 20 min, repeated 5 times). The combined filtrates were neutralized with DIEA, and the solution was evaporated. DMF was added (2 volumes) followed by the addition of MTBE to initiate phase separation. The mixture was centrifuged, and the supernatant was removed, leaving Fmoc-Rink-(AEEA)₈-NH₂ as an oily material. The final Fmoc group was removed with 30% piperidine in DMF followed by addition of MTBE to initiate phase separation. The supernatant was removed to give the title compound as an oil. ESMS m/z 739.5 (M+2H⁺/2)

### Example 3

### Preparation of 2-[2-[2-[[2-[2-[2-[[2-[2-[2-[[2-[2-[2-[[2-[2-[2-[[2-[2-[2- [[2-[2-[2-[[2-[2-[2-[[2-[2-[2-[[2-[2-[2-[[2-[4-[amino-(2,4-dimethoxyphenyl)methyl]phenoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetamide[Rink-(AEEA)₁₀-NH₂]

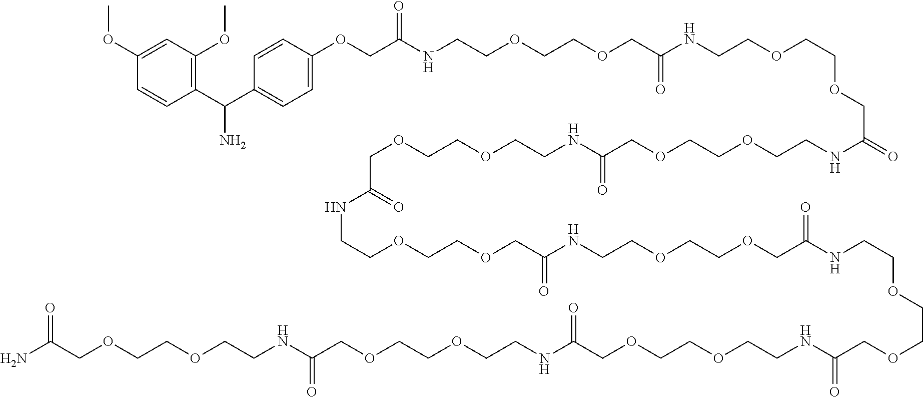 [SMILES: COc1ccc(C(N)c2ccc(OCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)cc2)c(OC)c1]

The title compound was prepared by solid-phase synthesis using the procedure essentially as described in Example 1, coupling 10 AEEA units onto the solid support before coupling on the Fmoc-Rink linker. ESMS m/z 1768.8 (M+H⁺)

### Example 4

### Preparation of 2-[2-[2-[[2-[2-[2-[[2-[2-[2-[[2-[2-[2-[[2-[4-[amino-(2,4-dimethoxyphenyl)methyl]phenoxy]acetyl]amino]ethoxy]ethoxy]acetyl]amino]ethoxy]ethoxy]acetyl]amino]ethoxy]ethoxy]acetyl]amino]ethoxy]ethoxy]acetamide[Rink-(AEEA)₄-NH₂]

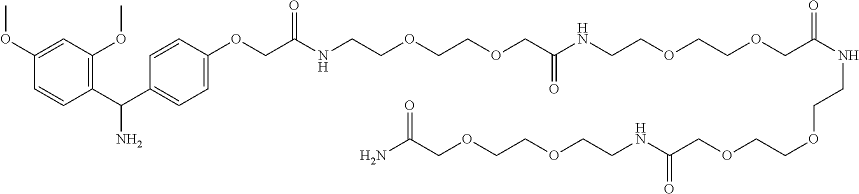 [SMILES: COc1ccc(C(N)c2ccc(OCC(=O)NCCOCCOCC(=O)NCCOCCOCC(=O)NCCOCCOCC(=O)NCCOCCOCC(N)=O)cc2)c(OC)c1]

The title compound was prepared by solid-phase synthesis using the procedure essentially as described in Example 1, coupling four AEEA units onto the solid support before coupling on the Fmoc-Rink linker. ESMS m/z 897.4 [(M+H⁺].

### Example 5

### Preparation of 2-[2-[2-[[2-[2-[2-[[2-[4-[amino-(2,4-dimethoxyphenyl)methyl]phenoxy]acetyl]amino]ethoxy]ethoxy]acetyl]amino]ethoxy]ethoxy]acetamide[Rink-(AEEA)₂-NH₂]

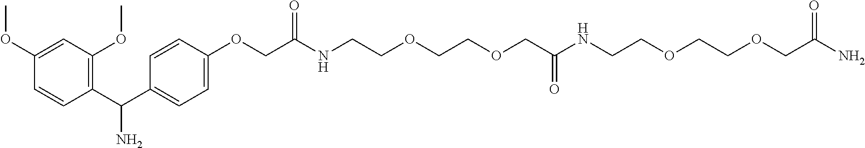 [SMILES: COc1ccc(C(N)c2ccc(OCC(=O)NCCOCCOCC(=O)NCCOCCOCC(N)=O)cc2)c(OC)c1]

The title compound was prepared by solid-phase synthesis using the procedure essentially as described in Example 1, coupling two AEEA units onto the solid support before coupling on the Fmoc-Rink linker. ESMS m/z 607.3 [(M+H⁺].

### Example 6

### Preparation of N-[2-[2-[2-[2-[2-[2-[2-[2-[2-[2-[2-[2-[2-[2-[2-[2-[2-[2-[2-[2-[2-[2-[2-[2-[2-[2-[2-[2-[2-(2-amino-2-oxo-ethoxy)ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethyl]-4-[4-(hydroxymethyl)-3-methoxy-phenoxy]butanamide[HMPB-(AEEA)₁₀-NH₂]

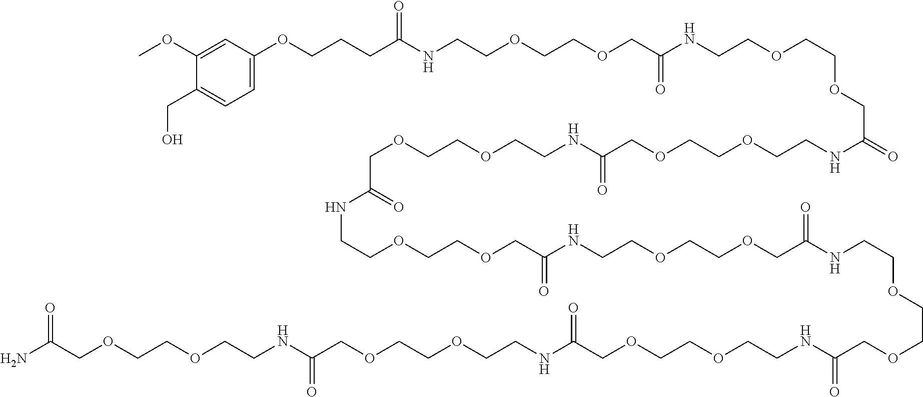 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1CO]

Using the procedure essentially as described in Example 1, 10 AEEA units were coupled onto Sieber resin. After the last AEEA unit was coupled onto the resin, it was washed with DCM and dried under a flow of nitrogen for 4 h. A portion of the resin (0.50 mmol) was deprotected with 20% piperidine in DMF essentially as described in Example 1, then the resin was suspended in DCM (11.4 mL) and TFA was added (0.6 mL). The suspension was mixed for 30 min at 25° C., then filtered and washed with DCM. The filtrate was neutralized with DIEA and concentrated under reduced pressure. The resultant oil was dissolved in DMSO (2 mL) and then MTBE was added (30 mL). The mixture was centrifuged, and the supernatant was removed, leaving (AEEA)₁₀-NH₂ as an oil in the centrifuge tube.

To the centrifuge tube containing (AEEA)₁₀-NH₂ was added a solution of DEPBT (150 mg, 0.50 mmol), HMPB (120 mg, 0.50 mmol), and DIEA (0.174 mL, 129 mg, 1.0 mmol) in DMSO (1.5 mL) allowing the solution to sit for 5 min before adding it to the tube. The resultant mixture was placed on a shaker and mixed for 2 h, and then MTBE (20 mL) was added to induce phase separation. The mixture was centrifuged (2500 rpm for 3 min) and the supernatant was discarded. Fresh MTBE (20 mL) was added to the tube and centrifuged (2500 rpm for 3 min) and the supernatant was discarded, leaving the title compound as an oil. ESMS m/z 1712.70 (M+Na-1H).

### Example 7

### Preparation of N-(17-amino-8,17-dioxo-3,6,12,15-tetraoxa-9-azaheptadecyl)-4-(4-(hydroxymethyl)-3-methoxyphenoxy)butanamide[HMPB-(AEEA)₂-NH₂]

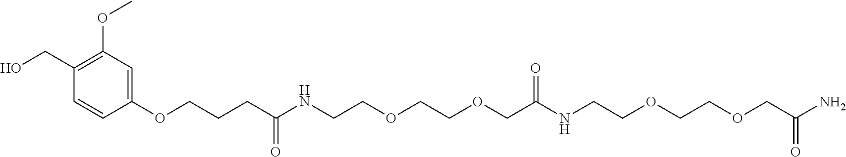 [SMILES: COc1cc(OCCCC(=O)NCCOCCOCC(=O)NCCOCCOCC(N)=O)ccc1CO]

The title compound was prepared essentially as described in Example 6, except HMPB was coupled to (AEEA)₂-NH₂ to give HMPB-(AEEA)₂-NH₂. ESMS m/z 552.3 (M+Na⁺).

### Example 8

### Preparation of N-(35-amino-8,17,26,35-tetraoxo-3,6,12,15,21,24,30,33-octaoxa-9,18,27-triazapentatriacontyl)-4-(4-(hydroxymethyl)-3-methoxyphenoxy)butanamide[HMPB-(AEEA)₄-NH₂]

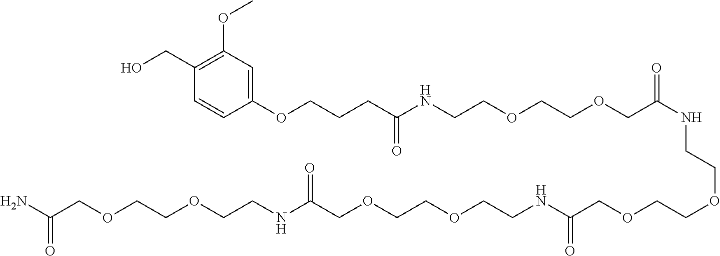 [SMILES: COc1cc(OCCCC(=O)NCCOCCOCC(=O)NCCOCCOCC(=O)NCCOCCOCC(=O)NCCOCCOCC(N)=O)ccc1CO]

The title compound was prepared essentially as described in Example 6, except HMPB was coupled to (AEEA)₄-NH₂ to give HMPB-(AEEA)₄-NH₂. ESMS m/z 842.4 (M+Na⁺).

### Example 9

### Preparation of N-(53-amino-8,17,26,35,44,53-hexaoxo-3,6,12,15,21,24,30,33,39,42,48,51-dodecaoxa-9,18,27,36,45-pentaazatripentacontyl)-4-(4-(hydroxymethyl)-3-methoxyphenoxy)butanamide[HMPB-(AEEA)₆-NH₂]

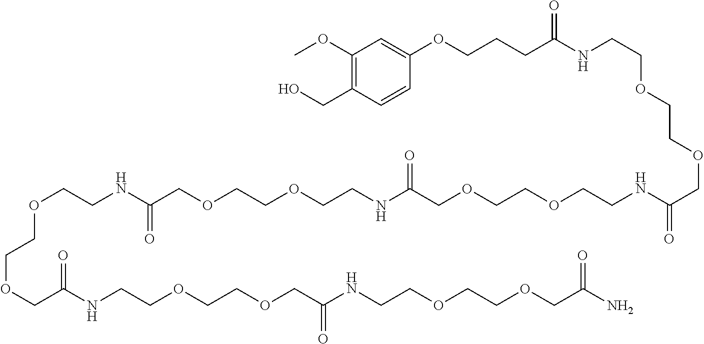 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1CO]

The title compound was prepared essentially as described in Example 6, except HMPB was coupled to (AEEA)₆-NH₂ to give HMPB-(AEEA)₆-NH₂. ESMS m/z 1132.5 (M+Na⁺).

### Example 10

### Preparation of 2-((5-amino-10,11-dihydro-5H-dibenzo[a,d][7]annulen-3-yl)oxy)-N-(53-amino-8,17,26,35,44,53-hexaoxo-3,6,12,15,21,24,30,33,39,42,48,51-dodecaoxa-9,18,27,36,45-pentaazatripentacontyl)acetamide[Ramage-(AEEA)₆-NH₂]

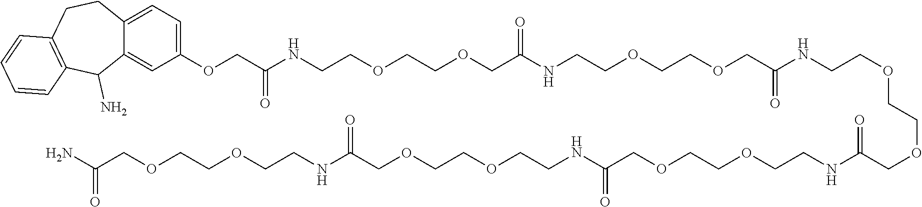 [SMILES: NC(=O)COCCOCCNC(=O)COCCOCCNC(=O)COCCOCCNC(=O)COCCOCCNC(=O)COCCOCCNC(=O)COCCOCCNC(=O)COc1ccc2c(c1)C(N)c1ccccc1CC2]

Coupling Ramage group onto (AEEA)₆ on Sieber resin: A portion of Fmoc-(AEEA)₆ on Sieber resin (986.8 mg, 0.5 mmol was swelled with DMF (10 mL over 20 min, repeated 3 times), deprotected using 20% piperidine in DMF (10 mL over 20 min, repeated three times), then washed with DMF (10 mL over 2 min, repeated 5 times). A solution of Fmoc-Suberol (0.76 g, 1.5 mmol, 3.0 equiv) in DMF:DMSO (4:1, 5 mL) was added to the reaction vessel, followed by Oxyma (0.750 M in DMF, 2 mL, 1.5 mmol, 3.0 equiv) and DIC (0.660 M in DMF, 2.5 mL, 1.65 mmol, 3.3 equiv) and mixed by nitrogen bubbling for 4 h. The reaction vessel was drained, washed with DMF (10 mL over 2 min, repeated 5 times), then deprotected using 20% piperidine in DMF (10 mL over 20 min, repeated 3 times). The resin was washed with DMF (10 mL over 2 min, repeated 5 times). After the final DMF wash, the resin was washed with DCM (10 mL over 2 min, repeated 5 times) and dried under a flow of nitrogen for 4 h to give 1.21 g Rink-(AEEA)₆ on Sieber resin. Cleavage of Ramage-(AEEA)₆-NH₂ from Sieber resin: Ramage-(AEEA)₆-NH₂ was cleaved from Sieber resin essentially as described in Example 1, except that 2% TFA in DCM was used and the reaction was stirred for 10 min instead of 30 min before further processing. ESMS m/z 1153.55 (M+H).

### Example 11

### Preparation of 2-[2-[2-[[2-[2-[2-[[2-[2-[2-[[2-[2-[2-[[2-[2-[2-[[2-[2-[2- [2-[2-[2-[[2-[2-[2-[2-[2-[2-[[2-[2-[2-[[2-[(11-amino-6,11-dihydro-5H-dibenzo[1,2-e:1′,2′-f][7]annulen-2-yl)oxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetamide[Ramage-(AEEA)₁₀-NH₂]

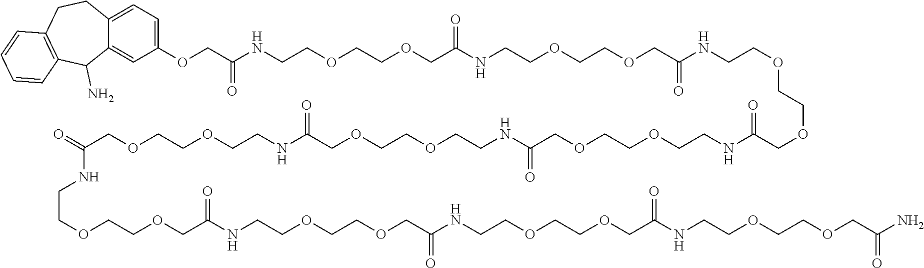 [SMILES: NC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COc1ccc2c(c1)C(N)c1ccccc1CC2]

The title compound was prepared by solid-phase synthesis using the procedure described in Example 10, coupling Fmoc-Suberol to (AEEA)₁₀ on Sieber resin. ESMS m/z 1733.8 (M+H).

### Example 12

### Preparation of 2-((5-amino-10,11-dihydro-5H-dibenzo[a,d][7]annulen-3-yl)oxy)-N-(35-amino-8,17,26,35-tetraoxo-3,6,12,15,21,24,30,33-octaoxa-9,18,27-triazapentatriacontyl)acetamide[Ramage-(AEEA)₄-NH₂]

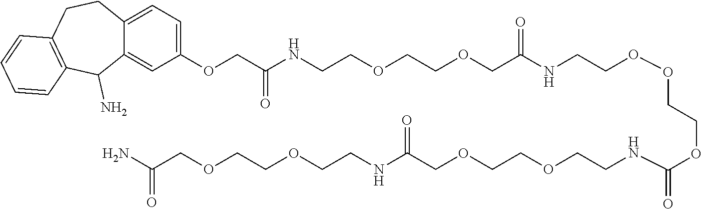 [SMILES: NC(=O)COCCOCCNC(=O)COCCOCCNC(=O)OCCOOCCNC(=O)COCCOCCNC(=O)COc1ccc2c(c1)C(N)c1ccccc1CC2]

The title compound was prepared by solid-phase synthesis using the procedure described in Example 10, coupling Fmoc-Suberol to (AEEA)₄ on Sieber resin. ESMS m/z 863.4 (M+H).

### Example 13

### Preparation of 2-((5-amino-10,11-dihydro-5H-dibenzo[a,d][7]annulen-3-yl)oxy)-N-(17-amino-8,17-dioxo-3,6,12,15-tetraoxa-9-azaheptadecyl)acetamide[Ramage-(AEEA)₂-NH₂]

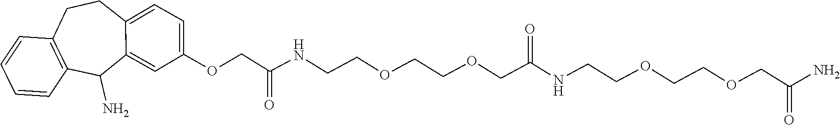 [SMILES: NC(=O)COCCOCCNC(=O)COCCOCCNC(=O)COc1ccc2c(c1)C(N)c1ccccc1CC2]

The title compound was prepared by solid-phase synthesis using the procedure described in Example 10, coupling Fmoc-Suberol to (AEEA)₂ on Sieber resin. ESMS m/z 595.3 (M+Na).

### Example 14

### Preparation of N-(2-(2-(2-amino-2-oxoethoxy)ethoxy)ethyl)-2-(2-(2-(2-(4-(hydroxymethyl)phenoxy)acetamido)ethoxy)ethoxy)acetamide[HMPA-(AEEA)₂-NH₂]

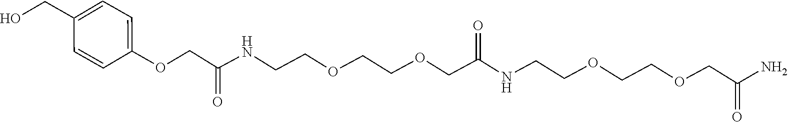 [SMILES: NC(=O)COCCOCCNC(=O)COCCOCCNC(=O)COc1ccc(CO)cc1]

Coupling HMPA group onto (AEEA)₂ on Sieber resin: A portion of Fmoc-(AEEA)₂ on Sieber resin (845 mg, 0.5 mmol) was swelled with DMF (10 mL over 20 min, repeated 3 times), deprotected using 20% piperidine in DMF (10 mL over 20 min, repeated three times), then washed with DMF (10 mL over 2 min, repeated 5 times). A solution of HMPA (91 mg, 0.5 mmol, 1.0 equiv), DIEA (0.174 mL, 1.0 mmol, 2.0 equiv) and DEPBT (150 mg, 0.5 mmol, 1.0 equiv) in DMF (10 mL) was added to the reaction vessel and mixed by nitrogen bubbling for 2 h. The reaction vessel was drained, washed with DMF (10 mL over 2 min, repeated 5 times), then washed with DCM (10 mL over 2 min, repeated 5 times) and dried under a flow of nitrogen for 4 h to give 908.6 mg HMPA-(AEEA)₂ on Sieber resin.

Cleavage of HMPA-(AEEA)₂-NH₂ from Sieber resin: HMPA-(AEEA)₂-NH₂ was cleaved from Sieber resin essentially as described in Example 1 to give the title compound. ESMS m/z 472.2 (M+H⁺).

### Example 15

### Preparation of 2-[2-[2-[[2-[2-[2-[[2-[2-[2-[[2-[2-[2-[[2-[2-[2-[[2-[2-[2- [[2-[2-[2-[[2-[2-[2-[[2-[2-[2-[[2-[2-[2-[[2-[4-(hydroxymethyl)phenoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetamide[HMPA-(AEEA)₁₀-NH₂]

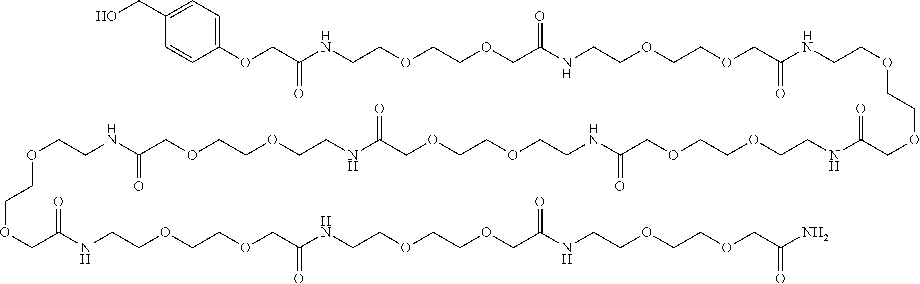 [SMILES: NC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COCCOCCNC(=O)COc1ccc(CO)cc1]

The title compound was prepared by solid-phase synthesis using the procedure described in Example 14, coupling HMPA to (AEEA)₁₀ on Sieber resin. ESMS m/z 1632.7 (M+H).

### Example 16-Liquid Phase Peptide Synthesis with Rink-(AEEA)₆-NH₂ and “Hard” Cleavage from Linker

The 19-mer peptide of SEQ ID NO: 1 was prepared using liquid phase peptide synthesis as follows.

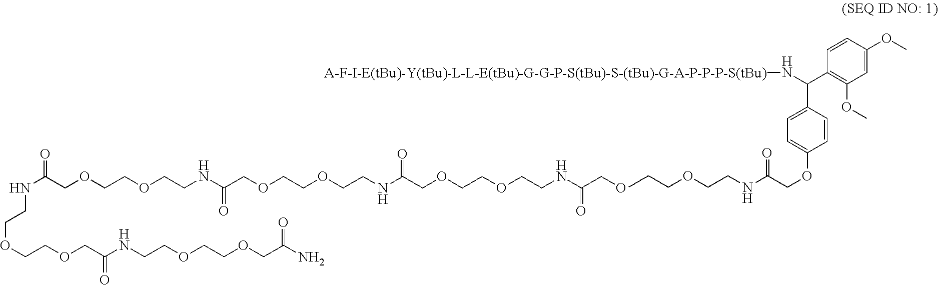

Elongation of amino acid chain on Rink-(AEEA)₆-NH₂: Fmoc-Ser (tBu)-OH (0.5 mmol), PyOxim (0.5 mmol), and DIEA (1 mmol) were dissolved in DMF (1 mL). The solution was mixed for 1 min and Rink linker-(AEEA)₆-NH₂ (prepared in Example 1, 0.16 mmol) was then added. The reaction solution was mixed for 10 min and then MTBE was added (5 mL). The mixture was centrifuged at 3250 rpm and the supernatant was discarded. The remaining material was mixed with 30% piperidine in DMF (1 mL) for 5 min. After mixing 5 min, MTBE (5 mL) was added. The mixture was centrifuged, and the supernatant was discarded. LCMS confirmed the absence of the product in the MTBE-containing supernatant in each centrifuge step.

The amino acid coupling and deprotection steps were repeated, coupling the Fmoc-protected amino acids (side chain —OH and —CO₂H groups protected with tBu) in order from C-terminus to N-terminus as given in SEQ ID NO: 1. DMF and DMSO were interchangeable as reaction solvent for the amino acid coupling and deprotection steps when the peptide construct was less than or equal to 15 amino acids. DMSO was preferred as the reaction solvent when the peptide length was greater than 15 amino acids. Upon completion of the final alanine residue coupling in SEQ ID NO: 1, the final Fmoc group was removed using the 30% piperidine/DMF deprotection reaction conditions described above. The product was lyophilized to give the peptide of SEQ ID NO: 1 (0.612 mg with residual solvent). ESMS m/z 1697.7 (M+2H⁺/2), 1132.0 (M+3H⁺/3). The crude isolated weight supports the LCMS data indicating that no product is lost in the supernatant during the phase separation steps with MTBE during the liquid phase synthesis.

“Hard” cleavage of peptide from linker: To remove the soluble linker and protecting groups, the peptide of SEQ ID NO: 1 was stirred in a solution containing TFA:triisopropylsilane:1,2-ethanedithiol:water (85:5:5:5 v/v ratio) for 2 h at 25° C., which gives the peptide with the sequence:

(SEQ ID NO: 2)
AFIEYLLEGGPSSGAPPPS-NH₂

The peptide of SEQ ID NO: 2 was precipitated with MTBE (10:1 MTBE compared to reaction volume), centrifuge as described above, then dried the in-vacuo. High-resolution MS m/z observed 944.4783 (charge state+2, neutral mass 1886.9426), theoretical neutral mass 1886.9414.

### Example 17-Liquid-Phase Peptide Synthesis with Ramage-(AEEA)₁₀-NH₂ and “Soft” Cleavage from Linker

Preparation of SEQ ID NO: 3 on Ramage-(AEEA)₁₀-NH₂: Using the procedure essentially as described in Example 16, the peptide of SEQ ID NO: 3 was prepared by liquid phase peptide synthesis starting with coupling Fmoc-Ser (tBu)-OH onto 3.0 mmol of Ramage-(AEEA)₁₀-NH₂, followed by Fmoc deprotection. The remaining Fmoc-protected amino acids (side chain —OH groups protected with tBu) were coupled/deprotected in order from C-terminus to N-terminus as given in SEQ ID NO: 3. Pro(7) and Pro(8) were incorporated as a dimer (Fmoc-Pro-Pro-OH). PyBOP could be used interchangeably with PyOxim. ESMS m/z 913.1 (M+3H⁺/3).

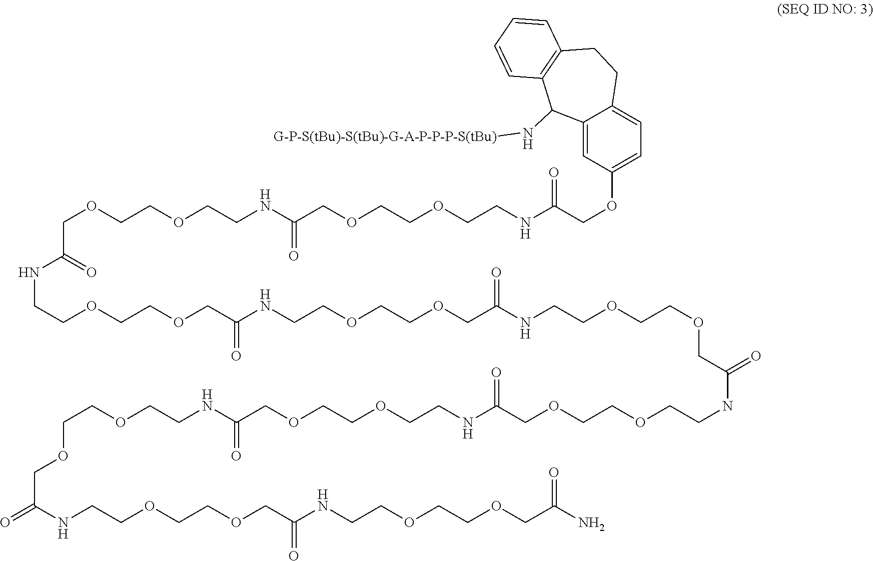

Soft cleavage of peptide from linker: To the peptide of SEQ ID NO: 3 was added 2% TFA in DCM (10 volumes) and the reaction was incubated for 30 min at room temperature. The reaction was neutralized using one equivalent of pyridine and the reaction mixture was concentrated in vacuo. Starting material was observed in the product, to which was added 20 volumes of 2% TFA in DCM. The mixture was incubated for 30 min at room temperature, neutralized with pyridine, and concentrated in vacuo again. To the residue was added 5% TFA in DCM solution (50 volumes). After 40 min, the solution was neutralized with pyridine concentrated under reduced pressure to give the crude peptide of SEQ ID NO: 4. ESMS m/z 1042.50 (M+Na+).

(SEQ ID NO: 4)
G-P-S(tBu)-S(tBu)-G-A-P-P-P-S(tBu)-NH₂

### Example 18-Liquid Phase Peptide Synthesis with Rink-(AEEA)₂-NH₂

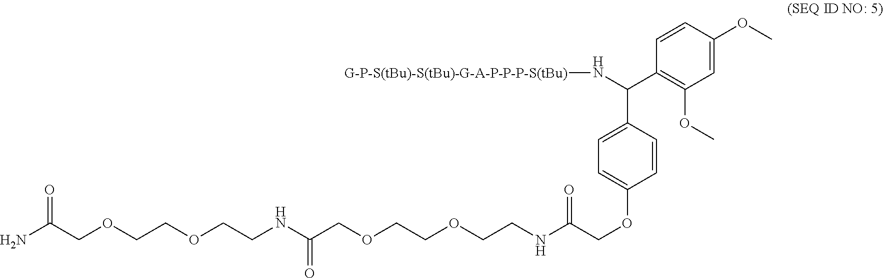

The peptide of SEQ ID NO: 5 was prepared using Rink-(AEEA)₂-NH₂ as the support in the liquid phase peptide synthesis essentially as described in Example 16. MTBE was added to the final Fmoc deprotection reaction mixture, then the mixture was centrifuged. The supernatant was discarded giving the peptide of SEQ ID NO: 5 as the oil sediment. ESMS m/z 1631.8 (M+Na⁺), 1609.8 (M+H⁺), 805.5 (M+2H⁺/2).

### Example 19-Liquid Phase Peptide Synthesis with HMPA-(AEEA)₁₀-NH₂ and “Soft” Cleavage from the Linker

The peptide of SEQ ID NO: 6 was prepared using liquid phase peptide synthesis as follows.

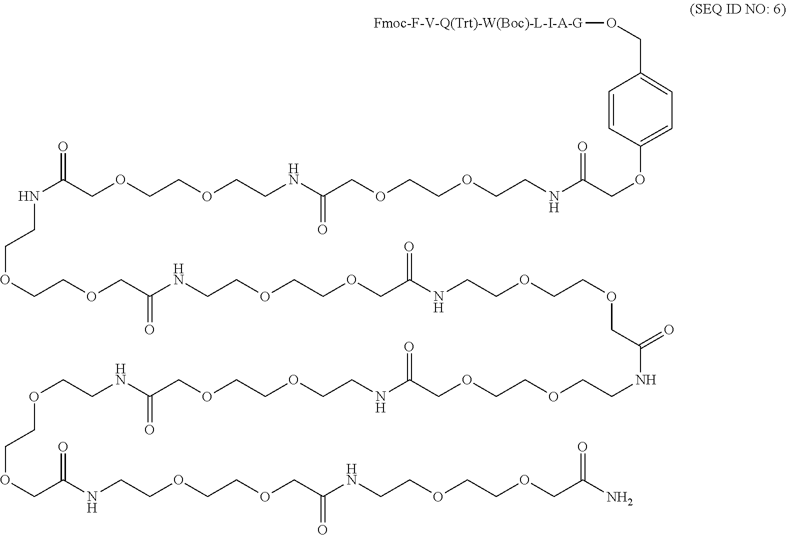

Elongation of amino acid chain on HMPA-(AEEA)₁₀-NH₂: To a solution of Fmoc-Gly-OH (0.2388 g, 0.8032 mmol) in DMSO (2 mL) was added PyBOP (0.418 g, 0.803 mmol) and DIEA (0.207 g, 1.60 mmol). It was mixed for 1 min and added to HMPA-(AEEA)₁₀-NH₂ (0.4372 g, 0.2678 mmol) and mixed for 90 min followed by addition of MTBE (40 mL). The mixture was centrifuged at room temperature, 3000 rpm for 3 min and supernatant was decanted. This washing was repeated 3 times leaving behind the bottom oil layer. The coupling procedure was repeated once more, then the resulting oil was mixed with 10% piperidine in DMF (2 mL) for 15 min. MTBE was added (20 mL) and the resulting mixture was centrifuged in a similar manner as described above. The 10% piperidine/DMF deprotection procedure was performed once more giving the bottom oil layer.

The remaining Fmoc-protected amino acids (glutamine side chain —CONH₂ group protected with trityl, and tryptophan side chain —NH group protected with -Boc) were coupled/deprotected in the manner described above in order from C-terminus to N-terminus as given in SEQ ID NO: 6, with subsequent coupling reactions being stirred for 45 min instead of 90 min. After the final amino acid (Fmoc-Phe-OH) coupling reaction had stirred for 45 min, isopropylacetate was added to the mixture followed by centrifugation and washing with MTBE as described above giving the bottom oil layer as the title compound. ES/MS m/z 1556.10 (M+2H+/2).

Soft cleavage of peptide from linker: The peptide of SEQ ID NO: 6 was subjected to the soft cleavage procedure essentially as described in Example 17 using two 30-min incubations with 3% TFA in DCM to give the peptide of SEQ ID NO: 7. ES/MS m/z 1519.60 (M+Na+).

(SEQ ID NO: 7)
Fmoc-F-V-Q(Trt)-W(Boc)-L-I-A-G-OH

### Example 20-Liquid Phase Peptide Synthesis with HMPB-(AEEA)₁₀-NH₂ and “Soft” Cleavage from Linker

The peptide of SEQ ID NO: 8 was prepared using liquid phase peptide synthesis as follows.

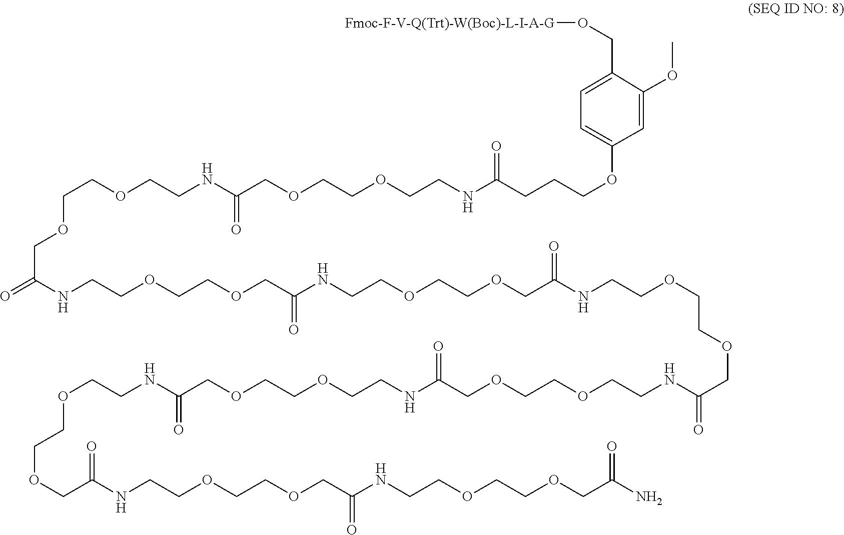

Elongation of amino acid chain on HMPB-(AEEA)₁₀-NH₂: The peptide of SEQ ID NO: 8 was prepared essentially as described in Example 16, coupling the Fmoc-protected amino acids (glutamine side chain —CONH₂ group protected with trityl, and tryptophan side chain —NH group protected with -Boc) and then deprotecting in the manner described in Example 16 in order from C-terminus to N-terminus as given in SEQ ID NO: 8 to give the peptide of SEQ ID NO: 8. ESMS m/z ESMS m/z 1585.70 (M+2H⁺/2).

Soft cleavage to obtain the peptide of SEQ ID NO: 7: The peptide of SEQ ID NO: 8 was dissolved in 3% TFA in DCM (20 volumes) and incubated for 30 min at room temperature. The reaction mixture was neutralized with one equivalent of pyridine and then washed with water. The organic layer was concentrated in vacuo to give the peptide of SEQ ID NO: 7. ESMS m/z 1497.7 (M+H+).

### Example 21

### Preparation of tert-butyl 20-[[(1S)-4-[2-[2-[2-[2-[2-[2-[[4-[4-[2-[2-[2-[2-[2-[2-[2-[2-[2-[2-[2-[2-[2-[2-[2-[2-[2-[2-[2-[2-[2-[2-[2-[2-[2-[2-[2-[2-[2-(2- amino-2-oxo-ethoxy)ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-4-oxo-butoxy]-2-methoxy-phenyl]methoxy]-2-oxo-ethoxy]ethoxy]ethylamino]-2-oxo-ethoxy]ethoxy]ethylamino]-1-tert-butoxycarbonyl-4-oxo-butyl]amino]-20-oxo-icosanoate [Fatty Acid Sidechain-HMPB-(AEEA)₁₀-NH₂]

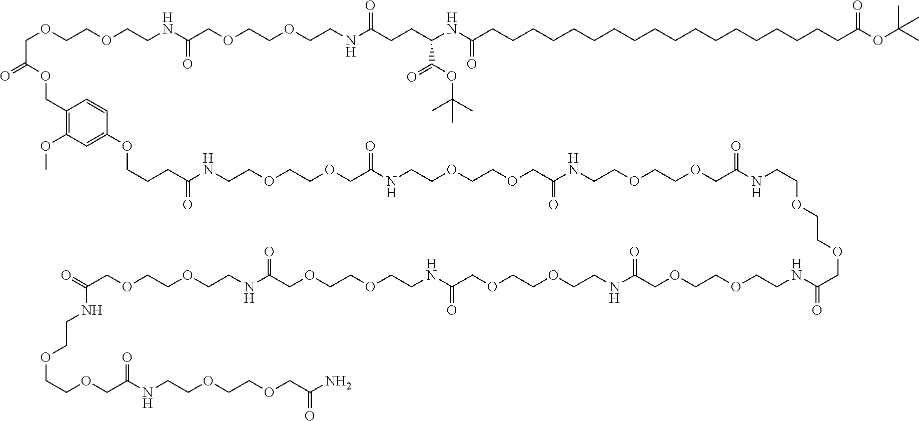 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1COC(=O)COCCOCCNC(=O)COCCOCCNC(=O)CC[C@H](NC(=O)CCCCCCCCCCCCCCCCCCC(=O)OC(C)(C)C)C(=O)OC(C)(C)C]

Method 1 [coupling order-(AEEA)₂, γ-Glu, fatty acid]: To a solution of Fmoc-(AEEA)₂-OH (4.493 g, 8.469 mmol) and DMAP (51.7 mg, 0.423 mmol) in DMSO (11 mL) was added DIC (1.33 mL, 8.469 mmol) and allowed to stand for 5 min. To the solution was added HMPB-(AEEA)₁₀-NH₂ (2.823 mmol), which was rinsed forward with DMSO (3 mL). The reaction solution was mixed for 2 h and then MTBE (45 mL) was added. The mixture was centrifuged at 3000 rpm for 2 min. The supernatant was decanted, and fresh MTBE (45 mL) was added, then the mixture was centrifuged at 3000 rpm for 2 min. The supernatant was discarded, and EtOAc (45 mL) was added, then the mixture was centrifuged at 3000 rpm for 2 minutes and then the supernatant decanted to afford an oil sediment. 30% Piperidine/DMF (8 mL) was added to the oil sediment and mixed for 15 minutes before addition of MTBE (45 mL). The mixture was centrifuged at 3000 rpm for 2 min. The supernatant was discarded, and fresh MTBE (45 mL) was added, then the mixture centrifuged again at 3000 rpm for 2 min. The supernatant was again decanted, leaving an oil sediment.

To a solution of Fmoc-Glu-OtBu (1.201 g, 2.823 mmol) and PyBOP (1.469 g, 2.823 mmol) in DMSO (9 mL) was added DIEA (0.983 mL, 5.64 mmol). The solution was allowed to age for 5 min and then added to the oil sediment and mixed for 30 min. MTBE (45 mL) was added, and the mixture centrifuged at 3000 rpm for 2 min. The supernatant was decanted, and fresh MTBE (45 mL) was added, and the mixture centrifuged at 3000 rpm for 2 min. The supernatant was decanted, and EtOAc (45 mL) was added, then the mixture centrifuged at 3000 rpm for 2 min. The supernatant was again decanted, leaving an oil sediment. 30% Piperidine/DMF (8 mL) was added to the oil sediment and mixed for 15 minutes before addition of MTBE (45 mL). The mixture was centrifuged at 3000 rpm for 2 min. The supernatant was discarded, and fresh MTBE (45 mL) was added, then the mixture centrifuged again at 3000 rpm for 2 min. The supernatant was again decanted, leaving an oil sediment.

To a solution of 20-tert-butoxy-20-oxo-icosanoic acid (1.13 g, 2.83 mmol, 1.0 equiv) and DEPBT (844.7 mg, 2.823 mmol, 1.0 equiv) in DMSO:toluene (9:1, 10 mL) was added DIEA (0.983 mL, 5.64 mmol, 2.0 equiv). The solution was allowed to age for 5 min and then added to the oil sediment and mixed for 30 min. MTBE (45 mL) was added, and the mixture centrifuged at 3000 rpm for 2 min. The supernatant was decanted, fresh MTBE (45 mL) was added, and the mixture centrifuged again at 3000 rpm for 2 min. The supernatant was again decanted and EtOAc (45 mL) was added, and the mixture centrifuged again at 3000 rpm for 2 min. The supernatant was discarded, leaving the title compound as the oil sediment. ESMS m/z 1274.10 (M+2H⁺/2).

Method 2 [coupling order-(AEEA)₂, succinimidyl ester of γ-Glu-fatty acid]: Fmoc-(AEEA)₂-OH was coupled to HMPB-(AEEA)₁₀-NH₂ as described above on the same scale. A solution of 30% piperidine/DMF (3 mL) was added to the oil of Fmoc-(AEEA)₂-HMPB-(AEEA)₁₀-NH₂ and mixed for 15 min. MTBE (40 mL total volume) was added to the reaction and the mixture was centrifuged (3000 rpm×2 min). MTBE was decanted and DMSO (2 mL) was added to the oil to dissolve it. Fresh MTBE (40 mL total volume) was added and the mixture was centrifuged once more and the MTBE layer decanted. O1-tert-butyl O5-(2,5-dioxopyrrolidin-1-yl)(2S)-2-[(20-tert-butoxy-20-oxo-icosanoyl)amino]pentanedioate (5.76 g, 8.46 mmol) was dissolved in a mixture of DMSO:toluene (9:1 ratio, 5.4 mL of DMSO+0.6 mL toluene). DIEA was added (3 mL, 16.92 mmol) and the resulting solution was allowed to stand for 5 min to pre-activate. The mixture was added to AEEA₂-HMPB-(AEEA)₁₀-NH₂ (2.82 mmol) in a centrifuge tube for 30 min. MTBE (40 mL total volume) was added, and the mixture was centrifuged (3000 rpm×2 min). The MTBE layer was removed, and a fresh MTBE (40 mL total volume) was added, and the mixture centrifuged once more. The supernatant was discarded, leaving the title compound as the oil. ESMS m/z 1273.70 (M+2H+/2).

### Example 22

### Preparation of 2-[2-[2-[[2-[2-[2-[[(4S)-5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetic acid [Fatty Acid Side Chain] by Soft Cleavage Method

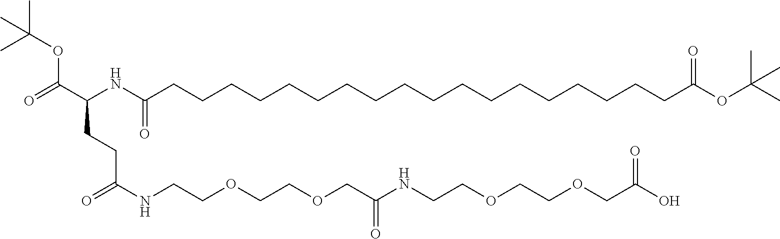 [SMILES: CC(C)(C)OC(=O)CCCCCCCCCCCCCCCCCCC(=O)N[C@@H](CCC(=O)NCCOCCOCC(=O)NCCOCCOCC(=O)O)C(=O)OC(C)(C)C]

Fatty acid side chain -HMPB-(AEEA)₁₀-NH₂ (prepared in Example 21, 2500 mg) was added to 2% TFA/toluene solution (10 volumes, 25 mL). The mixture was mixed for 10 min and then centrifuged at 3000 rpm for 5 min. The supernatant was collected and neutralized with pyridine (equimolar to TFA). To the remaining oily sediment was added MTBE (25 mL), and the mixture centrifuged at 3000 rpm for 5 min. The supernatant was collected, fresh MTBE (25 mL) was added to the oil, and the mixture was centrifuged again at 3000 rpm for 5 min. The supernatant was again collected, affording an oil sediment. The cleavage and washing were repeated twice more. The organics were washed with saturated aqueous NaCl and water, followed by concentration of the combined organics under reduced pressure to give the title compound. ESMS m/z 874 (M+H+). The crude product was analyzed using UPLC-CAD [column: Waters CSH™ C₁₈ 150×2.1 mm, 1.7 μm; column temperature: 50° C.; gradient—30 to 90% solvent B: solvent A over 21 min run; flow rate—0.5 mL/min; solvent A: 0.2% TFA in water; Solvent B: acetonitrile; detector: photodiode array UV, CAD]. The crude product showed a purity of 61.71%.

### Example 23

### Preparation of tert-butyl 20-[[(1S)-4-[2-[2-[2-[2-[2-[2-[[4-[4-[2-[2-[2-[2-[2-[2-[2-[2-[2-[2-[2-[2-[2-[2-[2-[2-[2-(2-amino-2-oxo-ethoxy)ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-4-oxo-butoxy]-2-methoxy-phenyl]methoxy]-2-oxo-ethoxy]ethoxy]ethylamino]-2-oxo-ethoxy]ethoxy]ethylamino]-1-tert-butoxycarbonyl-4-oxo-butyl]amino]-20-oxo-icosanoate [Fatty Acid Sidechain-HMPB-(AEEA)₆-NH₂]

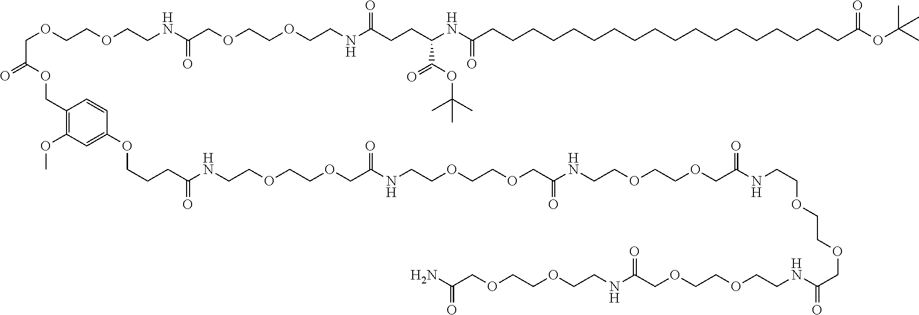 [SMILES: COc1cc(OCCCC(=O)NCCOCCOCC(=O)NCCOCCOCC(=O)NCCOCCOCC(=O)NCCOCCOCC(=O)NCCOCCOCC(=O)NCCOCCOCC(N)=O)ccc1COC(=O)COCCOCCNC(=O)COCCOCCNC(=O)CC[C@H](NC(=O)CCCCCCCCCCCCCCCCCCC(=O)OC(C)(C)C)C(=O)OC(C)(C)C]

To a solution of Fmoc-AEEA-OH (1156 mg, 3 mmol) and DMAP (18.4 mg, 0.149 mmol) in DMSO (4 mL) was added DIC (0.47 mL, 3 mmol). The resulting solution was allowed to stand for 5 min to activate and then added to HMPB-(AEEA)₆-NH₂ (1 mmol). The reaction mixture was placed on a shaker and mixed for 2 h. MTBE (40 mL total volume) was added to induce an oiling out and the mixture centrifuged (3000 rpm×2 min). The MTBE layer was discarded, and fresh MTBE (40 mL total volume) was added. The mixture centrifuged once more, and the MTBE was discarded. To the resulting oil, 30% piperidine/DMF (3 mL) was added and mixed for 15 min. MTBE (40 mL total volume) was then added, and the mixture was centrifuged (3000 rpm×2 min). The MTBE layer was decanted and DMSO (2 mL) was added to the oil to dissolve it and fresh MTBE (40 mL total volume) was added. The mixture was centrifuged once more, and the MTBE layer decanted. A solution of Fmoc-AEEA-OH (1156 mg, 3 mmol) and PyOxim (1598.1 mg, 3 mmol) was dissolved in DMSO (4 mL). DIEA was added (1 mL, 6 mmol) and the resulting solution was allowed to stand for 5 min for preactivation. The mixture was added to AEEA-HMPB-(AEEA)₆-NH₂ (1 mmol) in a centrifuge tube and allowed to mix for 30 min. Fmoc removal using 30% piperidine/DMF (3 mL) and MTBE washes were performed as described above. O1-tert-butyl O5-(2,5-dioxopyrrolidin-1-yl) (2S)-2-[(20-tert-butoxy-20-oxo-icosanoyl)amino]pentanedioate (2040 mg, 3 mmol) was dissolved in a mixture of DMSO:toluene (9:1 ratio, 2.7 mL of DMSO+0.3 mL toluene). DIEA was added (1 mL, 6 mmol) and the resulting solution was allowed to stand for 5 min for preactivation. The mixture was added to AEEA₂-HMPB-(AEEA)₆-NH₂ (1 mmol) in a centrifuge tube for 30 min. MTBE washing was then repeated to induce oil formation. The MTBE was discarded, leaving the title compound as the oil sediment. ESMS m/z 1966.3 (M+H+).

### Example 24

### Preparation of tert-butyl 20-[[(1S)-4-[2-[2-[2-[2-[2-[2-[[4-[2-[2-[2-[2-[2-[2-[2-[2-[2-[2-[2-[2-[2-[2-[2-[2-[2-[2-[2-[2-[2-[2-[2-[2-[2-[2-[2-[2-[2-[2-(2- amino-2-oxo-ethoxy)ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]ethoxy]ethylamino]-2-oxo-ethoxy]phenyl]methoxy]-2-oxo-ethoxy]ethoxy]ethylamino]-2-oxo-ethoxy]ethoxy]ethylamino]-1-tert-butoxycarbonyl-4-oxo-butyl]amino]-20-oxo-icosanoate [Fatty Acid Sidechain-HMPA-(AEEA)₁₀-NH₂]

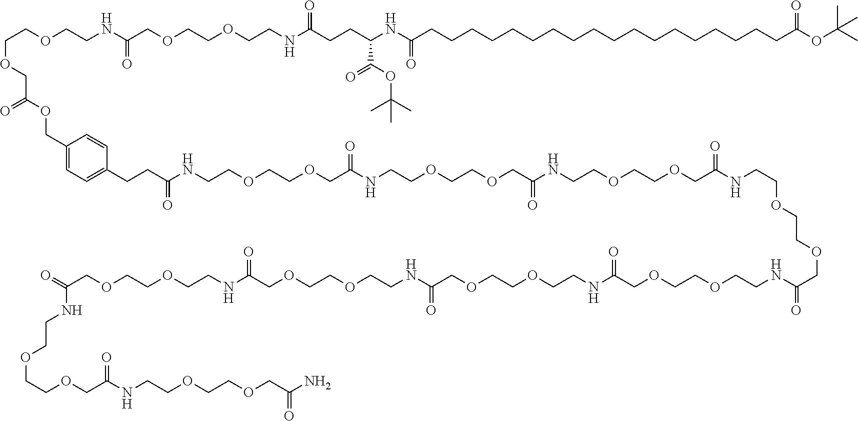 [SMILES: CC(C)(C)OC(=O)CCCCCCCCCCCCCCCCCCC(=O)N[C@@H](CCC(=O)NCCOCCOCC(=O)NCCOCCOCC(=O)OCc1ccc(CCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(=O)NCCOCCOCC(N)=O)cc1)C(=O)OC(C)(C)C]

To a solution of Fmoc-(AEEA)-OH (578 mg, 1.50 mmol) and DMAP (9.2 mg, 0.075 mmol) in DMSO (4 mL) was added DIC (0.235 mL, 1.50 mmol). The solution was aged for 5 min and then added to HMPA-(AEEA)₁₀-NH₂ (0.500 mmol) and mixed for 2 h. MTBE (14 mL) was added, and the mixture centrifuged at 3000 rpm for 2 min. The supernatant was decanted, and fresh MTBE (14 mL) was added, and the mixture again centrifuged at 3000 rpm for 2 min. The supernatant was discarded, and a second coupling cycle was performed as described above with Fmoc-(AEEA)-OH and after 2 h, MTBE (14 mL) was added, and the mixture centrifuged at 3000 rpm for 2 min. The supernatant was decanted, and fresh MTBE (14 mL) was added and again centrifuged at 3000 rpm for 2 min. The supernatant was discarded to give an oil sediment. 30% piperidine/DMF (3 mL) was added to the oil sediment and mixed for 15 min. MTBE (14 mL) was added, and the mixture centrifuged at 3000 rpm for 2 min. The supernatant was discarded, the oil sediment taken up in 1 mL of DMSO, and fresh MTBE (14 mL) was added. The mixture was again centrifuged at 3000 rpm for 2 min, and then the supernatant decanted to give an oily sediment.

To a solution of Fmoc-AEEA-OH (586 mg, 1.5 mmol) and PyOxim (791 mg, 1.5 mmol) in DMSO (4 mL) was added DIEA (0.523 mL, 3.0 mmol). The solution was allowed to age for 5 min and then added to the oily sediment obtained above and mixed for 30 min. MTBE (14 mL) was added, and the mixture centrifuged at 3000 rpm for 2 min. The supernatant was discarded, and fresh MTBE (14 mL) was added, and the mixture centrifuged once more at 3000 rpm for 2 min. The supernatant was discarded to give an oily sediment. 30% piperidine/DMF (3 ml) was added to the oil sediment and mixed for 15 min. MTBE (14 mL) was added, and the mixture centrifuged at 3000 rpm for 2 min, and then the supernatant discarded. Fresh MTBE (14 mL) was added, and the mixture again centrifuged at 3000 rpm for 2 min. The supernatant was discarded to afford (AEEA)₂-HMPA-(AEEA)₁₀-NH₂ the oily sediment.

In a manner similar to the second coupling of Fmoc-AEEA-OH, Fmoc-Glu-OtBu was coupled onto (AEEA)₂-HMPA-(AEEA)₁₀-NH₂, then MTBE addition and centrifugation, then deprotection with 30% piperidine in DMF followed by MTBE addition and centrifugation to give Fmoc-γGlu-(AEEA)₂-HMPA-(AEEA)₁₀-NH₂ as the oily sediment.

To a solution of 20-(tert-butoxy)-20-oxoicosanoic acid (600 mg, 1.5 mmol, 3.0 equiv) and PyBOP (781 mg, 1.5 mmol, 3.0 equiv) in DMSO (4 mL) was added DIEA (0.523 mL, 3.0 mmol, 6.0 equiv). The solution was allowed to age for 5 min, during which time the activated ester precipitated out, so toluene (8 mL) was added and then aged an additional 5 min. The solution was added to Fmoc-γGlu-(AEEA)₂-HMPA-(AEEA)₁₀-NH₂ and mixed for one hour (complete dissolution of the reaction mixture was not achieved). MTBE (14 mL) was added, and the mixture centrifuged at 3000 rpm for 2 min. The supernatant was discarded, and fresh MTBE (14 mL) was added, and the mixture centrifuged again at 3000 rpm for 2 min. The supernatant was discarded, leaving the title compound as an oil. ESMS m/z 2488.2 (M+H⁺); 1245.5 (M+2H⁺/2).

### Example 25-Liquid Phase Peptide Synthesis with HMPB-(AEEA)₁₀-NH₂ and “Soft” Cleavage from Linker

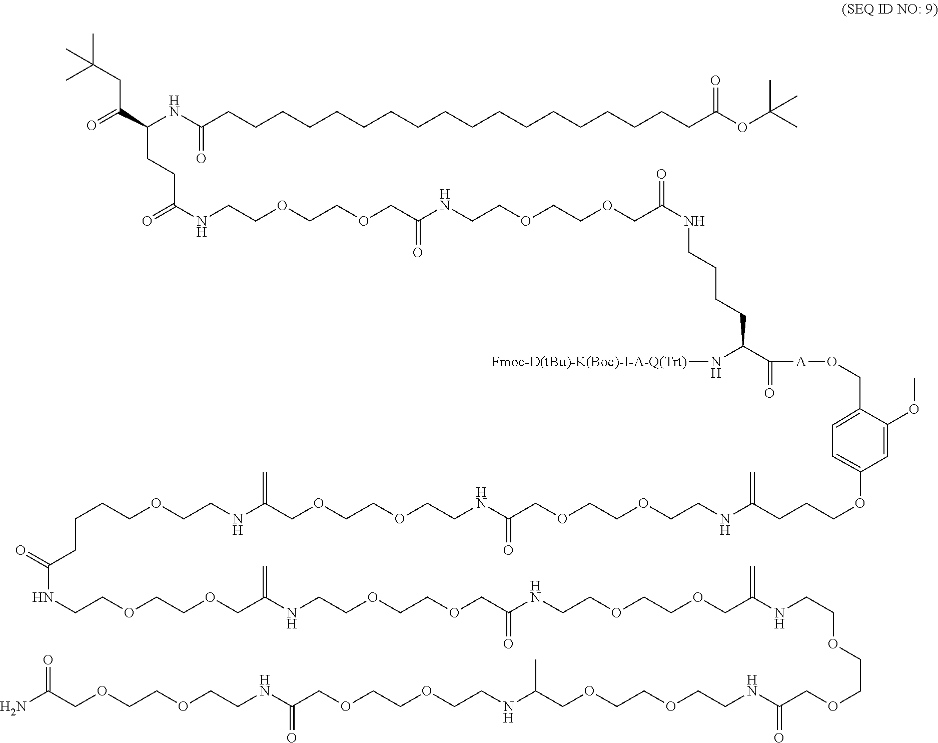

Elongation of amino acid chain on HMPB-(AEEA)₁₀-NH₂: A mixture of Fmoc-Ala-OH (1.9 g, 6 mmol) and DMAP (36.8 mg, 0.301 mmol) were dissolved in 4 mL of DMSO. DIC (0.93 mL, 6 mmol) was added to the mixture and allowed to stand for 5 min for preactivation. This mixture as added to HMPB-(AEEA)₂-NH₂ and the mixture was shaken for 2 h at room temperature. MTBE was added bringing the mixture volume to 40 mL, inducing a phase change with the coupling product Fmoc-Ala-HMPB-(AEEA)₂-NH₂ precipitating as an oil. The mixture was centrifuged (3000 rpm×2 min) and the MTBE supernatant was decanted. Additional MTBE was added to the oily sediment bring the volume up to 40 mL, and the mixture was centrifuged and the MTBE supernatant was decanted. The coupling procedure was repeated twice with Fmoc-Ala-OH to achieve complete coupling.

A solution of 30% piperidine in DMF (3 mL) was added to the resulting oily sediment and the mixture was shaken for 15 min. MTBE was then added to bring the volume up to 40 mL, causing an oil to precipitate. The mixture was centrifuged (3000 rpm×2 min), the supernatant was decanted, and DMSO (1 mL) was added to the oily sediment. MTBE was added to bring the volume to 40 mL, and the mixture was centrifuged again, and the supernatant decanted to leave the oily sediment.

The amino acid coupling and deprotection steps were repeated essentially as described in Example 16, first to couple (2S)-6-[[2-[2-[2-[[2-[2-[2-[[(4S)-5-tert-butoxy-4-[(20-tert-butoxy-20-oxo-icosanoyl)amino]-5-oxo-pentanoyl]amino]ethoxy]ethoxy]acetyl]amino]ethoxy]ethoxy]acetyl]amino]-2-(9H-fluoren-9-ylmethoxycarbonylamino) hexanoic acid, and then to couple the remaining Fmoc-protected amino acids (aspartic acid side chain —CO₂H group protected with tBu, glutamine side chain —NH₂ protected with trityl, and lysine —NH₂ protected with -Boc) in order from C-terminus to N-terminus as given in SEQ ID NO: 9, using DEPBT in place of PyOxim and mixing for 60 min for each coupling procedure to give the peptide of SEQ ID NO: 9. ESMS m/z 1961.08 (M+2H⁺/2).

### Soft Cleavage to Obtain the Peptide of SEQ ID NO: 10:

A mixture of the peptide of SEQ ID NO: 9 (0.38 mmol, 1.5 g) in 2% TFA in DCM (15 mL) was incubated at room temperature for 30 min. Pyridine (1 equivalent) was then added to neutralize the reaction mixture. The reaction mixture was washed with water and the organic layer was concentrated in vacuo. The residue was washed with EtOAc, then dissolved in DCM again and concentrated in vacuo to give the peptide of SEQ ID NO: 10. ESMS m/z 1125 (M+2H+/2).

The crude product was analyzed using UPLC-MS [column: Waters CSH™ C₁₈ 150×2.1 mm, 1.7 mm; column temperature: 50° C.; gradients-30 to 90% solvent B: solvent A over 21 min run; flow rate: 0.5 mL/min; solvent A: 0.2% TFA in water; Solvent B: acetonitrile; detector: photodiode array UV, ESMS]. The crude peptide showed a purity of 71.31%.

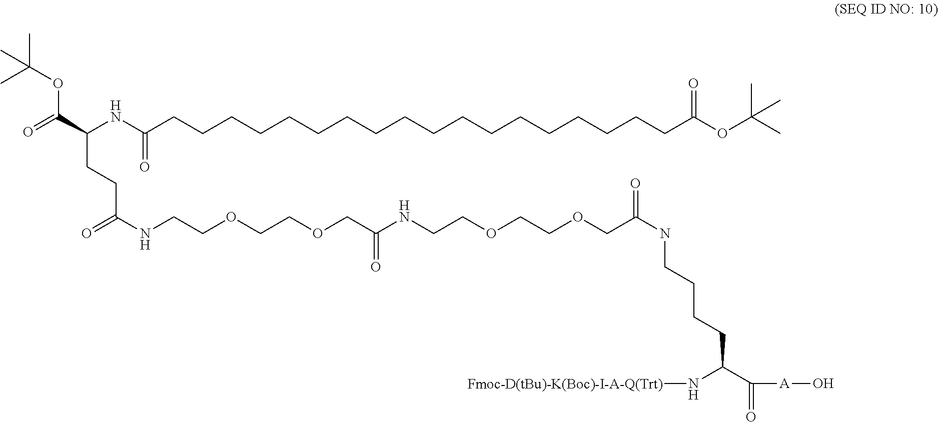

### Example 26-Liquid Phase Peptide Synthesis of with HMPB-(AEEA)₁₀-NH₂

The peptide of SEQ ID NO: 11 was prepared using liquid phase peptide synthesis as follows.

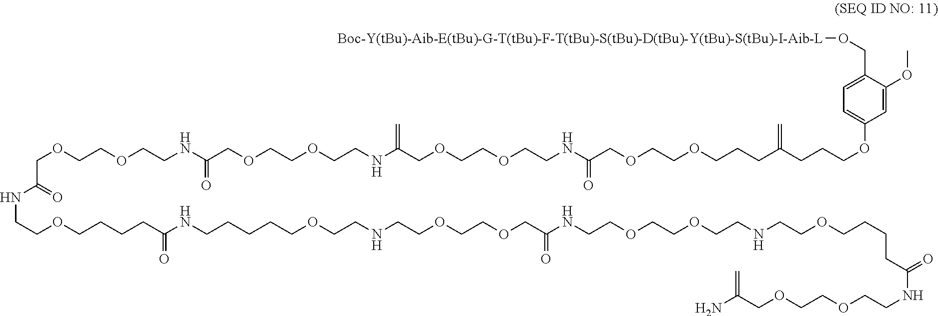

A mixture of Fmoc-Leu-OH (4.2 g, 12 mmol) and DMAP (73.6 mg, 0.602 mmol) was dissolved in DMSO (5 mL). DIC (1.58 g, 1.96 mL, 12 mmol), was added and the mixture as allowed to stand for 5 min for preactivation. The mixture was added to HMPB-(AEEA)₁₀-NH₂ (4 mmol) and shaken at room temperature for 2 h. MTBE was added bringing the mixture volume to 40 mL, inducing a phase change with the coupling product Fmoc-Leu-HMPB-(AEEA)₂-NH₂ precipitating as an oil. The mixture was centrifuged (3000 rpm×3 min) and the MTBE supernatant was decanted. Additional MTBE was added to the oily sediment bring the volume up to 40 mL, and the mixture was centrifuged and the MTBE supernatant was decanted. The coupling procedure was repeated twice with Fmoc-Leu-OH to achieve complete coupling.

A solution of 30% piperidine in DMF (3 mL) was added to the resulting oily sediment and the mixture was shaken for 15 min. MTBE was then added to bring the volume up to 40 mL, causing an oil to precipitate. The mixture was centrifuged (3000 rpm×3 min), the supernatant was decanted, and DMSO (1 mL) was added to the oily sediment. MTBE was added to bring the volume to 40 mL, and the mixture was centrifuged again, and the supernatant decanted to leave the oily sediment.

Subsequent amino acid coupling and deprotection steps were repeated essentially as described in Example 16, coupling Fmoc-protected amino acids (—OH and —CO₂H side chain groups protected with tBu) in order from C-terminus to N-terminus as given in SEQ ID NO: 11, to give the peptide of SEQ ID NO: 11. ESMS m/z 1893 (M+2H+/2), 1262 (M+3H+/3).

### Example 27-Liquid Phase Synthesis of Tetrameric Peptide and “Soft” Cleavage from HMPB-Based Linkers

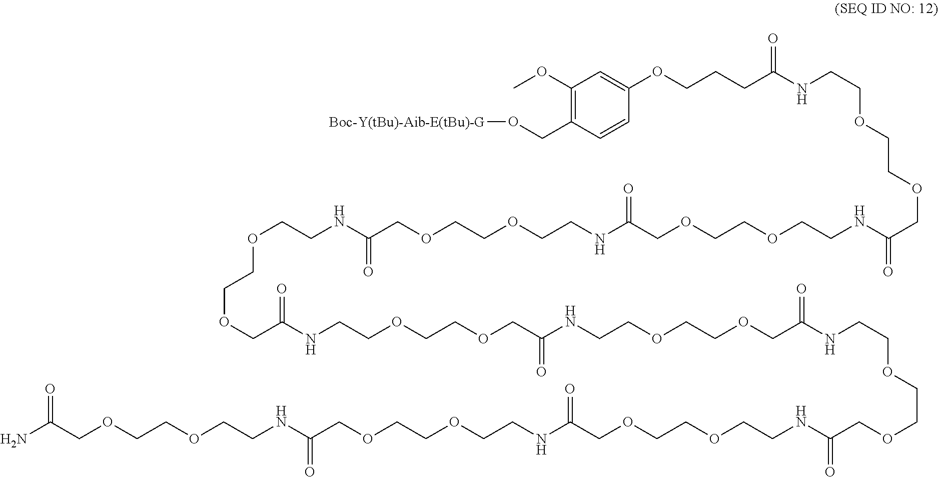

Tetrameric peptide preparation on HMPB-(AEEA)₁₀-NH₂: The peptide of SEQ ID NO: 12 was prepared essentially as described in Example 16 with the following changes: the first amino acid (Fmoc-Gly-OH) was coupled to HMPB-(AEEA)₁₀-NH₂ as follows, Fmoc-Gly-OH, DIC, and DMAP (3:3:0.15 molar ratio) were dissolved in DMSO and mixed for 1 min then added to HMPB-(AEEA)₁₀-NH₂. After 2 hours, MTBE (5 mL) was added to initiate phase separation. The mixture was centrifuged at 3250 rpm and the supernatant was discarded. A second coupling cycle was performed as described above with Fmoc-Gly-OH and after 2 h, MTBE (5 mL) was added to initiate phase separation. The mixture was centrifuged at 3250 rpm and the supernatant was discarded. The Fmoc group was removed using 30% piperidine/DMF, and then subsequent couplings and deprotections were performed essentially as described in Example 16 to give the peptide of SEQ ID NO: 12. ESMS m/z 2360.1 (M+Na⁺), 2338.1 (M+H⁺), 1169.6 (M+2H⁺/2).

Soft Cleavage of Tetrameric Peptide from HMPB-(AEEA)₁₀-NH₂ Linker:

Two methods were employed to “soft” cleave the peptide of SEQ ID NO: 12 to give the protected tetrameric peptide of SEQ ID NO: 13:

(SEQ ID NO: 13)
Boc-Y(tBu)-Aib-E(tBu)-G-OH.

Method 1: To the peptide of SEQ ID NO: 12 was added 5% TFA/DCM solution (10 volumes). After 30 min, the solution was neutralized with pyridine and washed twice with 10% NaCl solution. The organics were dried over Na₂SO₄ and concentrated under reduced pressure. The residue was dissolved in minimal DMF and diluted with water (3 volumes). The mixture was extracted three times with MTBE and the combined organics were concentrated under reduced pressure to give the peptide of SEQ ID NO: 13. ESMS m/z 687.4 (M+Na+), 665.4 (M+H+).

Method 2: To the peptide of SEQ ID NO: 12 was added a 2% TFA/toluene solution (10 volumes). The mixture was mixed for 10 minutes and then centrifuged at 3000 rpm for 5 minutes. The supernatant was collected and neutralized with pyridine (equimolar to TFA). To the remaining oily sediment was added MTBE (3 mL), and the mixture centrifuged at 3000 rpm for 5 min. The supernatant was collected, fresh MTBE (3 mL) was added to the oil, and the mixture was centrifuged again at 3000 rpm for 5 min. The supernatant was again collected, affording an oil sediment. The cleavage and washing were repeated twice more on the oil sediment. The combined organic supernatant mixture was washed with saturated aqueous NaCl and water followed by concentrating the combined organics under reduced pressure to give the peptide of SEQ ID NO: 13.

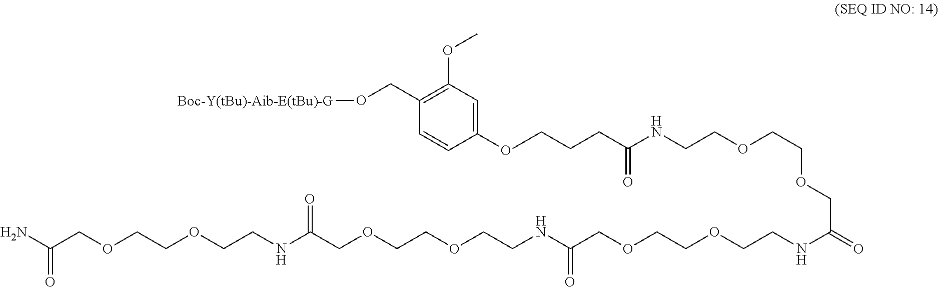

Tetrameric peptide preparation on HMPB-(AEEA)₄-NH₂: The peptide of SEQ ID NO: 14 was prepared essentially as described above using HMPB-(AEEA)₄-NH₂. ESMS m/z 1488.7 (M+Na⁺).

Soft cleavage of tetrameric peptide from HMPB-(AEEA)₄-NH₂ linker: To the peptide of SEQ ID NO: 14 was added 3% TFA/DCM solution (20 volumes). After 30 min, the solution was neutralized with pyridine and washed twice with water. The organics were dried over MgSO₄ and concentrated under reduced pressure to give the peptide of SEQ ID NO: 13. ESMS m/z 687.4 (M+Na+), 665.4 (M+H+).

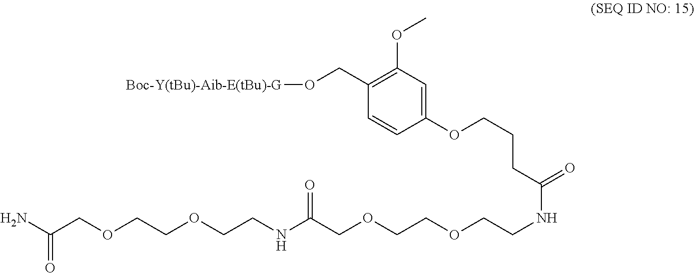

Tetrameric peptide preparation on HMPB-(AEEA)₂-NH₂: The peptide of SEQ ID NO: 15 was prepared essentially as described above using HMPB-(AEEA)₂-NH₂. ESMS m/z 1198.5 (M+Na⁺).

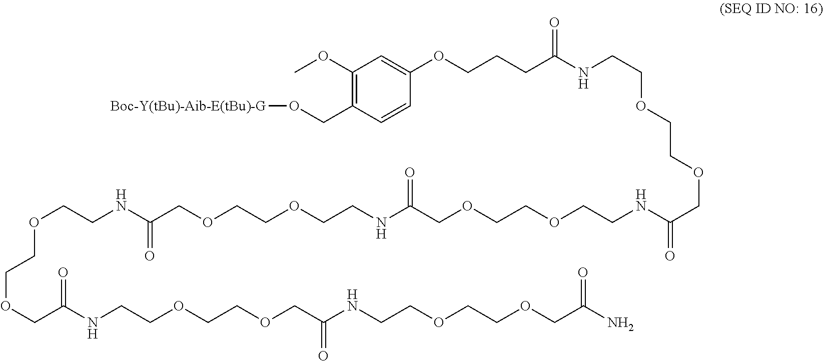

Tetrameric peptide preparation on HMPB-(AEEA)₆NH₂: The peptide of SEQ ID NO: 16 was prepared essentially as described above using HMPB-(AEEA)₆-NH₂. ESMS m/z 1778.8 (M+Na⁺).

### Example 28-Liquid Phase Synthesis of Tetrameric Peptide and “Soft” Cleavage from HMPA-Based Linkers

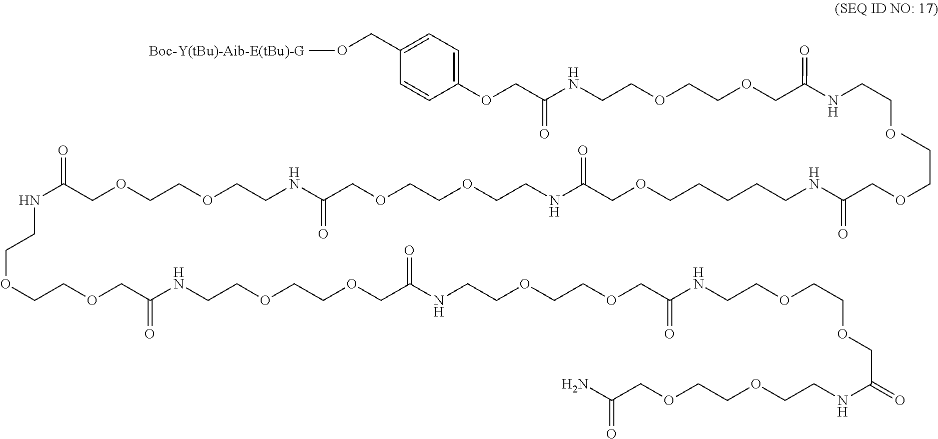

Tetrameric peptide preparation on HMPA-(AEEA)₁₀-NH₂: To a solution of Fmoc-Gly-OH (140 mg, 0.470891 mmol) in DMF (1 mL) was added DIC (60 mg, 0.47 mmol) and 2,4,6-trimethylpyridine (0.125 mL, 0.945 mmol). This solution was added to HMPA-(AEEA)₁₀-NH₂ (256 mg, 0.15679 mmol) and was shaken at room temperature for 2 h. MTBE (20 mL) was added to it and resulting mixture was centrifuged (5000 rpm×3 min). The supernatant was decanted and the extractive MTBE washing was performed three more times, separating the bottom oil layer from the supernatant by decantation each time. The above coupling and washing processes were repeated three times. To drive the reaction to completion. The sedimentary oil layer was mixed with 25% piperidine in DMF (2 mL) for 20 min. It was washed with MTBE (20 mL) and centrifuged in a similar manner as described above three times leaving behind the bottom oil layer.

To a solution of Fmoc-Glu(OtBu)-OH (90.74 mg, 0.2111 mmol) and PyOxim (112.5 mg, 0.2111 mmol) in DMSO (750 μL) and acetonitrile (750 μL), was added DIEA (74 μL, 0.424 mmol) and the solution was mixed for 2 min. This solution was added to the above oil sediment (0.180 g, 0.107 mmol) and mixed at room temperature for 30 min followed by addition of MTBE (20 mL). The mixture was centrifuged (3000 rpm×3 min) and the supernatant was decanted. This extractive washing was performed twice, and the supernatant was decanted and thus separated from bottom oil layer each time. 10% piperidine/DMF (2 mL) was added to the oil sediment and mixed for 15 min. It was washed with MTBE (20 mL) and centrifuged in a similar manner as described above two times leaving behind the bottom oil layer.

To a solution of Fmoc-Aib-OH (68.4 mg, 0.210 mmol) and PyOxim (110.8 mg, 0.2080 mmol) in DMSO (750 μL) was added DIPEA (55.05 μL, 0.316 mmol). The solution was mixed for 2 min and added to the above oil layer (0.197 g, 0.105 mmol) and allowed to mix for 30 min at room temperature followed by addition of MTBE (20 mL) causing an oil to precipitate. The mixture was centrifuged (3000 rpm×3 min) and the supernatant was decanted. This extractive washing was performed twice, separating the bottom oil layer from the supernatant by decantation each time. 10% Piperidine/DMF (2 mL) was added to the oily sediment and mixed for 15 min. It was similarly washed with MTBE (20 mL) and centrifuged as described above two times leaving behind the bottom oil layer.

To a solution of Boc-Tyr(tBu)-OH (106.4 mg, 0.3153 mmol) and PyOxim (166.3 mg, 0.3153 mmol) in DMSO (750 μL) and acetonitrile (750 μL), was added DIEA (91 μL, 0.316 mmol). The solution was mixed for 2 min and added to the above oil layer (201.0 mg, 0.1026 mmol,) and allowed to mix for 30 min at room temperature followed by addition of MTBE (20 mL) causing an oil to precipitate. The mixture was centrifuged (3000 rpm×3 min). This extractive washing was performed twice, separating the bottom oil layer from the supernatant by decantation each time. The above coupling and washing processes were performed 4 times to drive the reaction to completion giving the peptide of SEQ ID NO: 17 as an oil. ESMS m/z 2279.10 (M+H+); 1140.20 (M+2H+/2).

Soft cleavage of tetrameric peptide from HMPA-(AEEA)₁₀-NH₂ linker: To the peptide of SEQ ID NO: 17 (102.3 mg, 0.04488 mmol) was added 1% TFA (in DCM (1.22 mL) and the mixture was stirred at room temperature for 1 h. Pyridine (10.6 mg, 0.134 mmol) was added to the resulting solution. The mixture was washed with water (4.5 mL), then the organic layer was then collected and concentrated to give peptide of SEQ ID NO: 13 as an oil; LCMS indicated that the reaction had progressed to 33% conversion from starting material to product. ESMS m/z 665.40 (M+H+); 687.30 (M+Na+).

Tetrameric Peptide Preparation on HMPA-(AEEA)₁₀-NH₂:

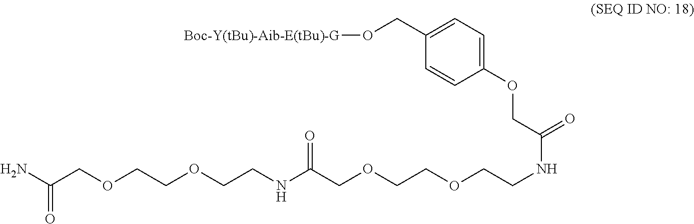

To a solution of Fmoc-Gly-OH (522.5 mg, 1.757 mmol) in DMSO (2.5 mL) added EDC (249.7 mg, 1.609 mmol) and ethyl cyanoglyoxylate-2-oxime (272.4 mg, 1.898 mmol). This solution was added to HMPA-(AEEA)₂ (0.4143 g, 0.8787 mmol) and the solution was mixed at room temperature for 2 h followed by addition of MTBE (30 mL) to precipitate an oil. The mixture was centrifuged (3000 rpm×3 min) and the supernatant was decanted. This extractive washing was performed three times, separating the bottom oil layer from the supernatant by decantation each time. The above coupling and washing processes were repeated three times to drive the reaction to completion. The sedimentary oil layer was mixed with 10% piperidine in DMF (2 mL) for 20 min. It was washed with MTBE (20 mL) and centrifuged in a similar manner as described above. The latter Fmoc removal step was performed once more giving the bottom oil layer.

To a solution of Fmoc-Glu(OtBu)-OH (0.842 g, 1.980 mmol) and PyOxim (1.055 g, 1.980 mmol) in DMSO (3 mL) and acetonitrile (1 mL), was added DIEA (0.516 g, 4.01 mmol) and the solution was mixed for 1 min. This solution was added to the above oil sediment (0.504 g, 0.954 mmol) and mixed at room temperature for 45 min followed by addition of MTBE (40 mL) to precipitate an oil. The mixture was centrifuged (3000 rpm×3 min) and the supernatant was decanted. This extractive washing was performed twice, and the supernatant was decanted and thus separated from bottom oil layer each time. The coupling and washing steps were repeated once more. 10% piperidine/DMF (2 mL) was added to the oil sediment and mixed for 15 min. It was washed in a similar manner as described above leaving behind the bottom oil layer.

To a solution of Fmoc-Aib-OH (970 mg, 2.98 mmol) and PyOxim (1.57 g, 2.92 mmol) in DMSO (3 mL) and acetonitrile (1 mL), was added DIEA (782 μL, 4.48 mmol) and the solution was mixed for 1 min. This solution was added to the above oil sediment (1.001 g, 1.402 mmol) and mixed at room temperature for 45 min, then the mixture was washed with MTBE and centrifuged as described above. 10% piperidine/DMF (2 mL) was added to the oil sediment and mixed for 15 min. The mixture was washed with MTBE and centrifuged as described above. The latter Fmoc-removal step was performed once more giving the bottom oil layer.

To a solution of Boc-Tyr(tBu)-OH (1.63 g, 4.83 mmol,) and PyOxim (2.55 g, 4.74 mmol) in DMSO (3 mL) and acetonitrile (1 mL), was added DIEA (1.13 mL, 6.48 mmol) and the solution was mixed for 1 min. This solution was added to the above oil sediment (1.289 g, 1.613 mmol) and mixed at room temperature for 60 min, then it was washed with MTBE and centrifuged as described above. The coupling and MTBE wash steps were repeated once more giving the peptide of SEQ ID NO: 18 as an oil. ESMS m/z 1118.50 (M+H+); 1140.50 (M+Na+).

Soft cleavage of tetrameric peptide from HMPA-(AEEA)₂-NH₂ linker: To the peptide of SEQ ID NO: 18 (50 mg, 0.045 mmol) was added a mixture of 1,1,1,3,3,3-hexafluoro-2-propanol (200 μL, 1.91 mmol) in DCM (0.8 mL) and the resulting mixture was stirred at room temperature for 20 min. ACN (2 mL) was added and the reaction mixture was concentrated in vacuo. The ACN addition and concentration steps were repeated twice. The residue was subjected to the above conditions (stirring in 1,1,1,3,3,3-hexafluoro-2-propanol and DCM for 20 min, followed by addition of ACN and concentrating) twice, giving the peptide of SEQ ID NO: 13 as an oil; LCMS indicated that the reaction had progressed to 8% conversion from starting material to product. ESMS m/z 664.40 (M+).

### Example 29—Liquid-Phase Fragment-Based Preparation of Peptide of SEQ ID NO: 22 Using Rink Linker-(AEEA)₆-NH₂

The synthesis of the peptide of SEQ ID NO: 22 via a liquid-phase fragment-based approach is herein described.

Preparation of the peptide of SEQ ID NO: 19 on Rink-(AEEA)₆-NH₂: Using the procedure essentially as described in Example 16, the peptide of SEQ ID NO: 19 was prepared by liquid phase peptide synthesis starting with coupling Fmoc-Ser (tBu)-OH onto 0.05 mmol of Rink-(AEEA)₆-NH₂, followed by Fmoc deprotection. The remaining Fmoc-protected amino acids (side chain —OH groups protected with tBu) were coupled/deprotected in order from C-terminus to N-terminus as given in SEQ ID NO: 19.

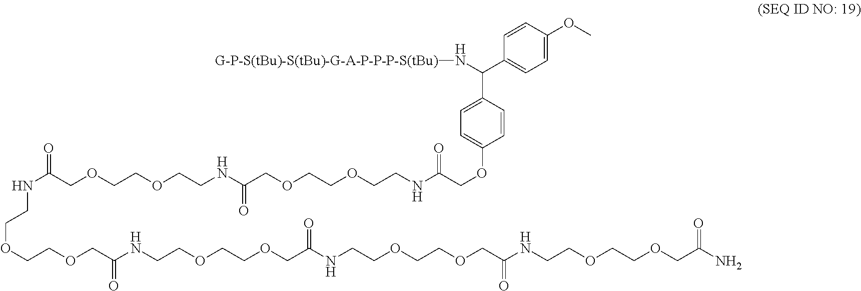

ESMS m/z 1095.7 (M+2H⁺/2), 731.0 (M+3H⁺/3).

Coupling of the peptide of SEQ ID NO: 7 onto the peptide of SEQ ID NO: 19: The peptide of SEQ ID NO: 7 was coupled onto the peptide of SEQ ID NO: 19 using the coupling procedure essentially as described in Example 16 using DMSO as reaction solvent to give the peptide of SEQ ID NO: 20. The reaction was sampled after 1 min reaction time and analyzed by LCMS, which shows that the reaction is complete.

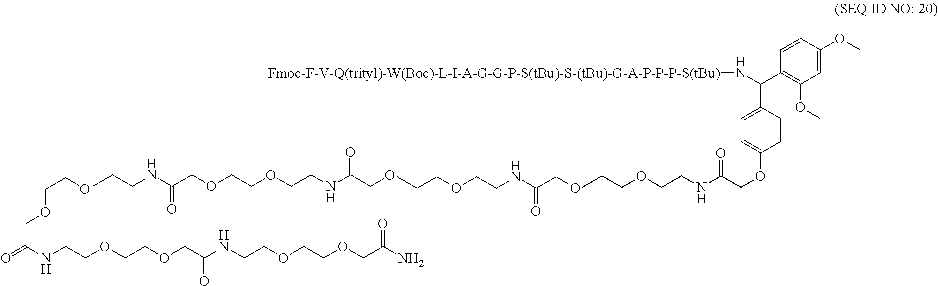

ESMS m/z 1835.90 (M+2H⁺/2), 1224.20 (M+3H⁺/3). The N-terminal Fmoc group was removed from the peptide of SEQ ID NO: 20 using the deprotection procedure essentially as described in Example 16 using DMSO as reaction solvent to give the peptide of SEQ ID NO: 21:

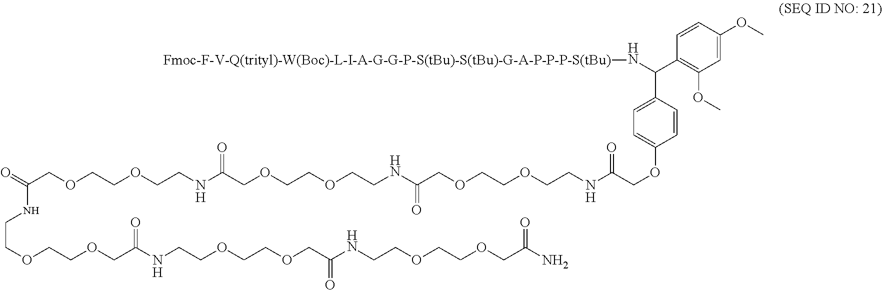

ESMS m/z 1724.9 (M+2H⁺/2), 1150.20 (M+3H⁺/3)

Coupling of the peptide of SEQ ID NO: 10 onto the peptide of SEQ ID NO: 21: the peptide of SEQ ID NO: 10 (0.06 mmol) was coupled onto the peptide of SEQ ID NO: 21 using the coupling procedure essentially as described in Example 16 using DMSO as reaction solvent, and then the Fmoc group was removed using the deprotection procedure essentially as described in Example 16 using 30% piperidine in DMSO to give the peptide of SEQ ID NO: 22.

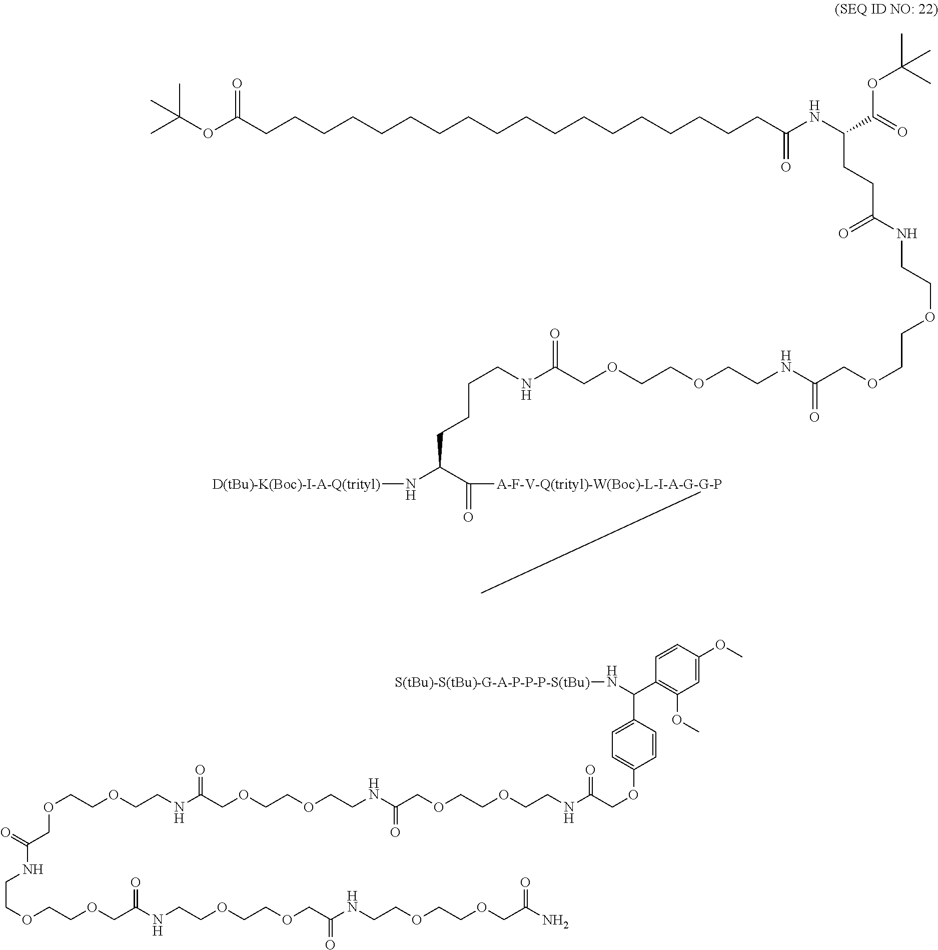

ESMS m==1820.1 (M+3H⁺/3), 1365.3 (M+4H⁺/4).

### Example 30

### Preparation of the Peptide of SEQ ID NO: 23 with Rink-(AEEA)₁₀-NH₂ Using a Fragment-Based Approach

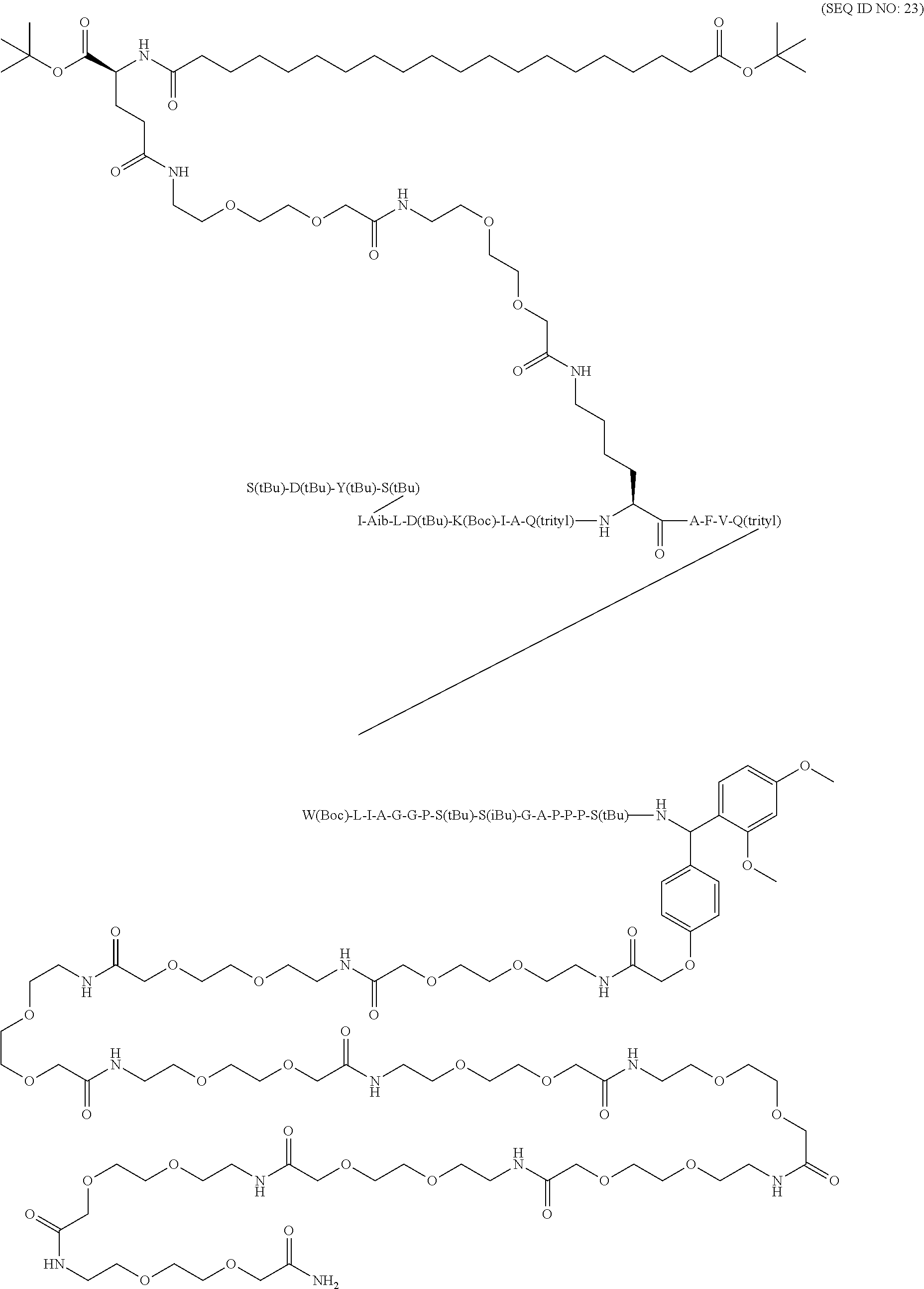

To a solution of Fmoc-P-P-P-S(tBu)-OH (SEQ ID NO: 24, 0.2291 g, 0.3395 mmol) in DMSO (2 mL) was added PyBOP (0.1781 g, 0.3422 mmol) and DIEA (54.186 μL, 0.311 mmol). It was mixed for 1 min and added to Rink-(AEEA)₁₀-NH₂ (0.500 g, 0.338 mmol) and mixed for 60 min followed by addition of MTBE (12 mL) giving an oily precipitate. The mixture was centrifuged at room temperature (3000 rpm×3 min) and the supernatant was decanted. This washing was repeated one more time. The oily sediment was washed with isopropyl acetate (twice, 10 mL each time) in a similar manner. The coupling was repeated twice more. The oil layer was mixed with 20% piperidine in DMF (2 mL) for 20 min and washed with MTBE (12 mL) and isopropyl acetate in a similar manner giving an oily sediment.

To a solution of Fmoc-S(tBu)-S(tBu)-G-A-OH (SEQ ID NO: 25, 0.2262 g, 0.3454 mmol) in DMSO (2 mL) was added PyBOP (0.180 g, 0.346 mmol) and DIEA (60.34 μL, 0.346 mmol). The solution was mixed for 1 min and then added to the oily sediment from the previous step (0.5073 g, 0.2303 mmol) and mixed for 60 min. MTBE (12 mL) was added, precipitating an oil, and the mixture was centrifuged (3000 rpm×3 min) and the supernatant was decanted. The oily sediment was washed with twice with MTBE and isopropyl acetate as described above. The coupling reaction was repeated twice more. The oil layer was mixed with 20% piperidine in DMF (2 mL) for 20 min and precipitated and washed/centrifuged with MTBE (12 mL) and isopropyl acetate in a similar manner giving an oily sediment.

In a manner essentially as described for the previous coupling and deprotection, the following were coupled sequentially onto the oily sediment isolated in the previous step:
1.

(SEQ ID NO: 26)
Fmoc-A-G-G-P-OH,

2.

(SEQ ID NO: 27)
Fmoc-Q(Trt)-W(Boc)-L-I-OH,

3.

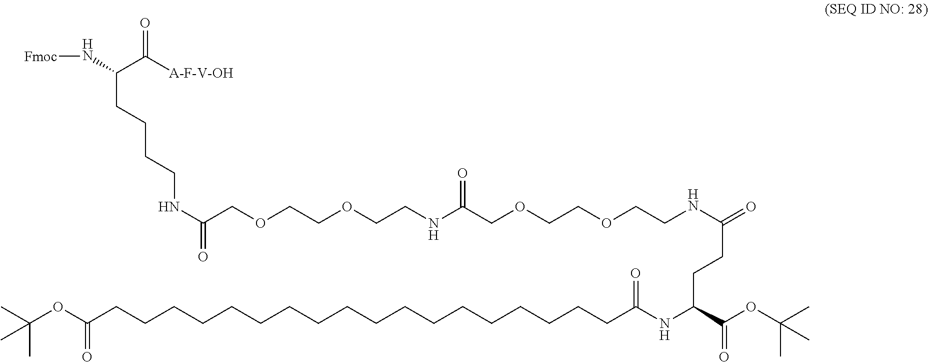

4.

(SEQ ID NO: 29)
Fmoc-K-I-A-Q(Trt)-OH

5.

(SEQ ID NO: 30)
Fmoc-I-Aib-L-D(Bu)-OH

6.

(SEQ ID NO: 31)
Fmoc-S(tBu)-D(tBu)-Y(tBu)-S(tBu)-OH

After coupling the peptide of SEQ ID NO: 31, the piperidine/DMF deprotection step was performed, and after the MTBE/isopropyl acetate washing/centrifuging procedures the peptide of SEQ ID NO: 23 was obtained as an oil. ESMS m/z 1756.50 (M+4H+/4).

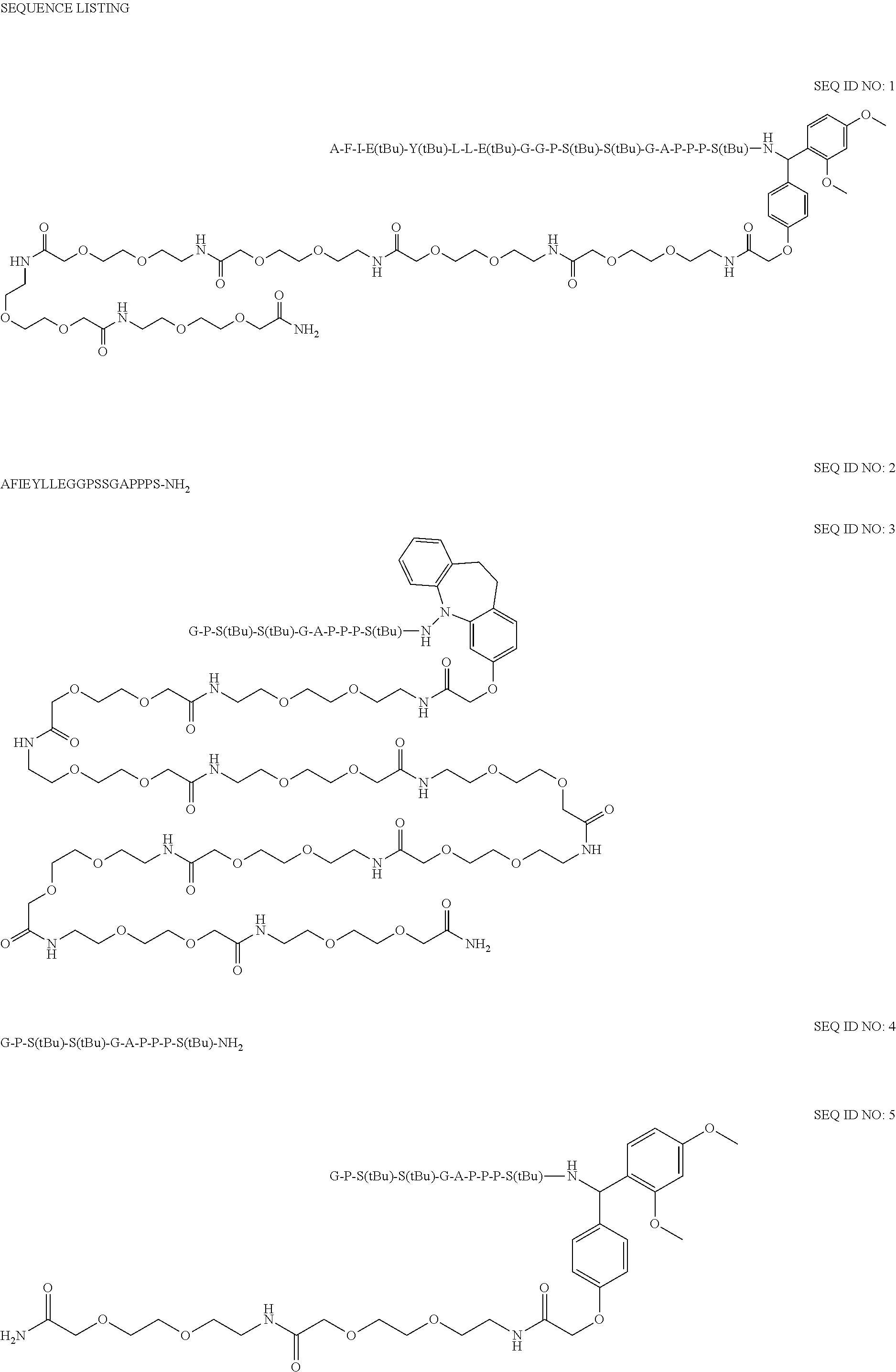

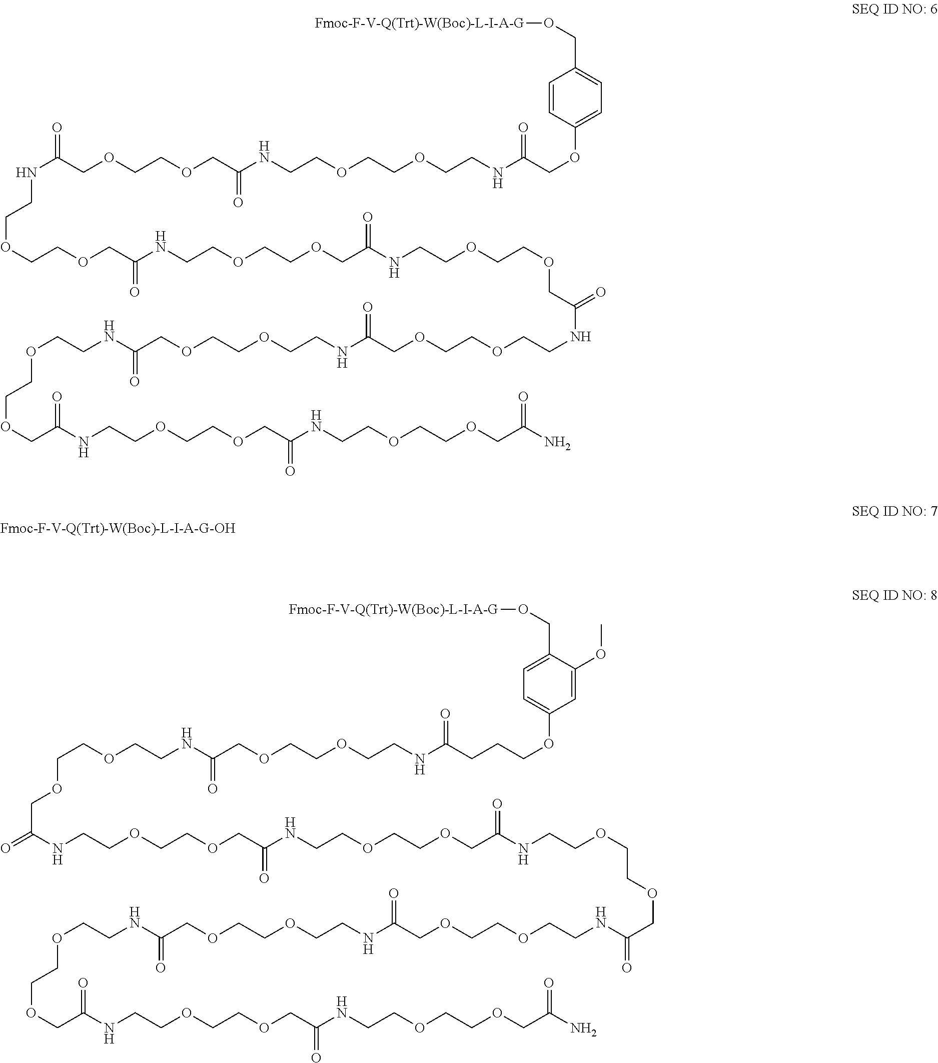

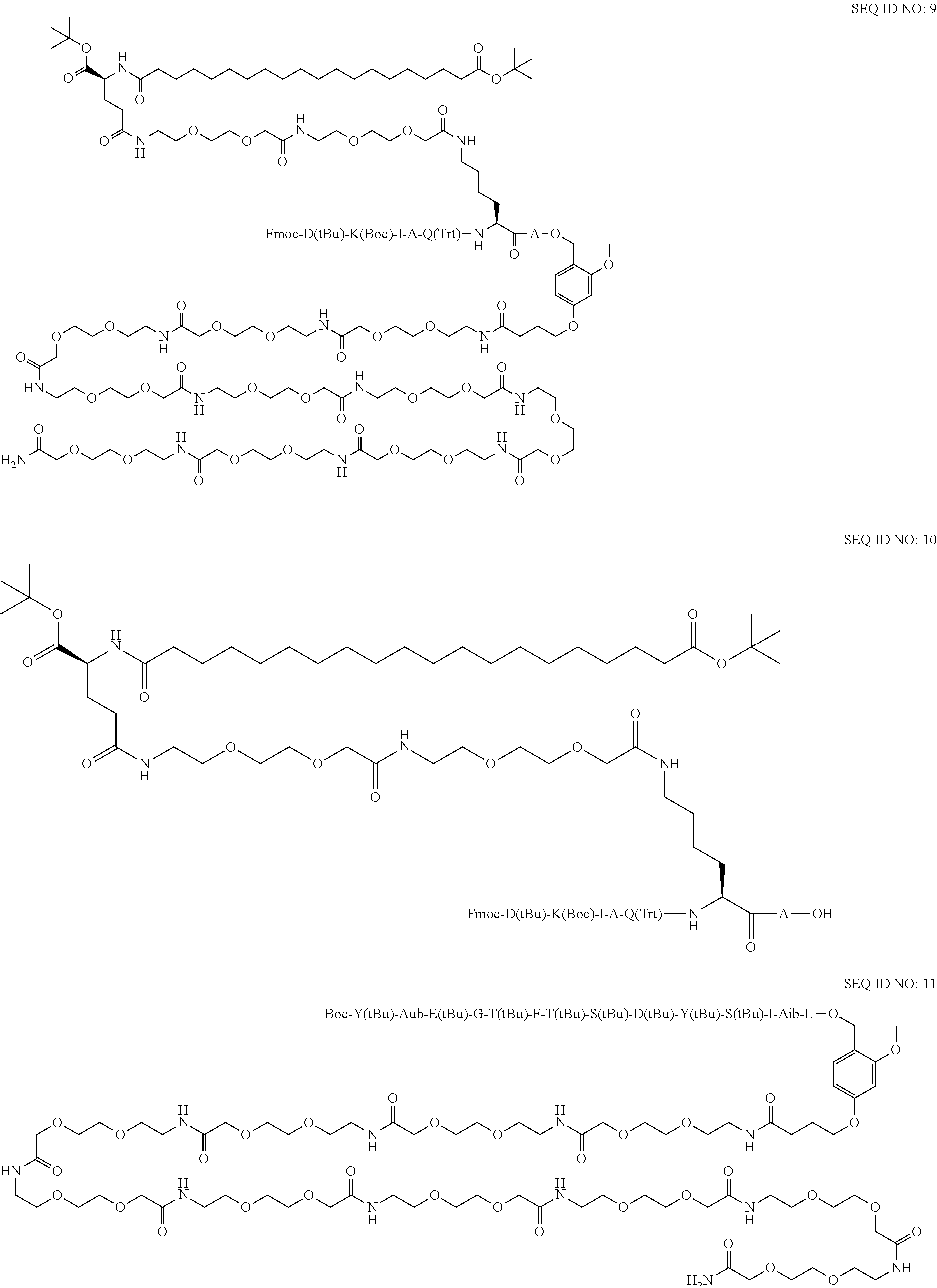

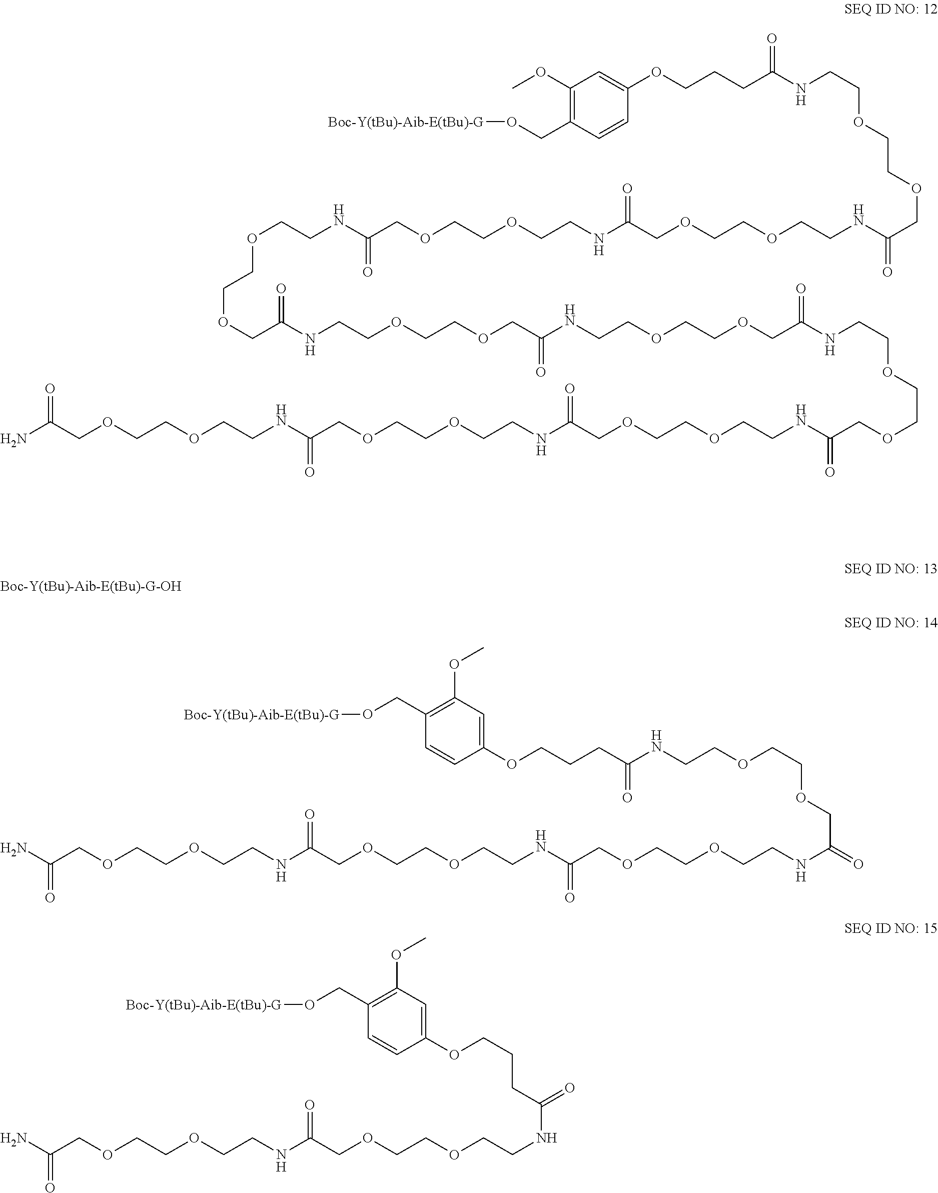

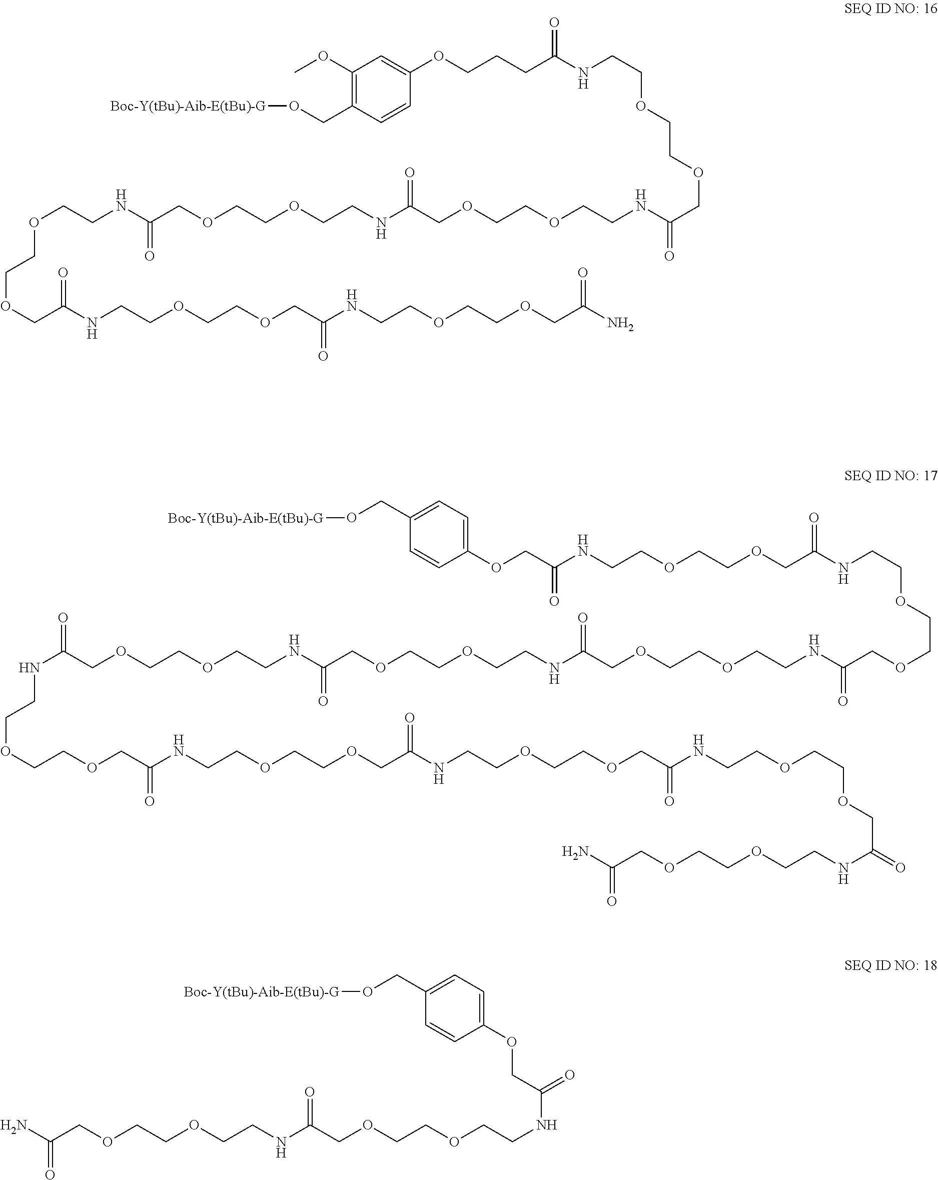

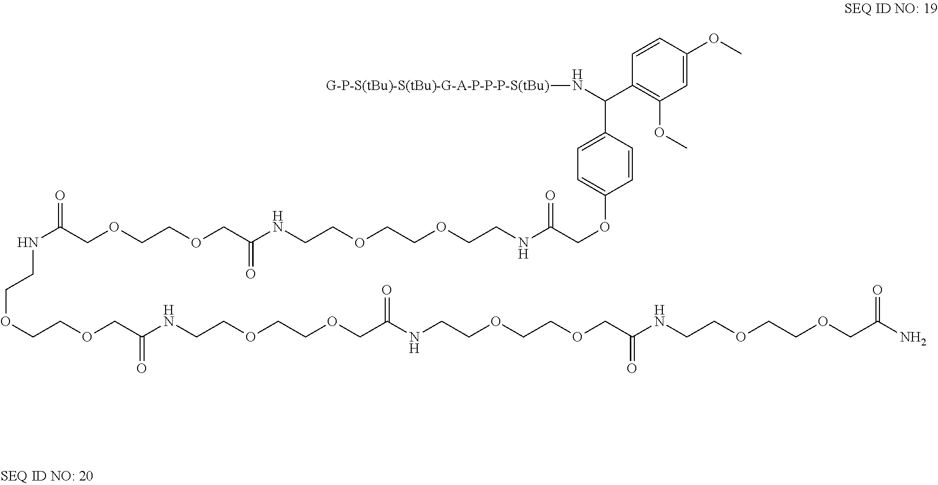

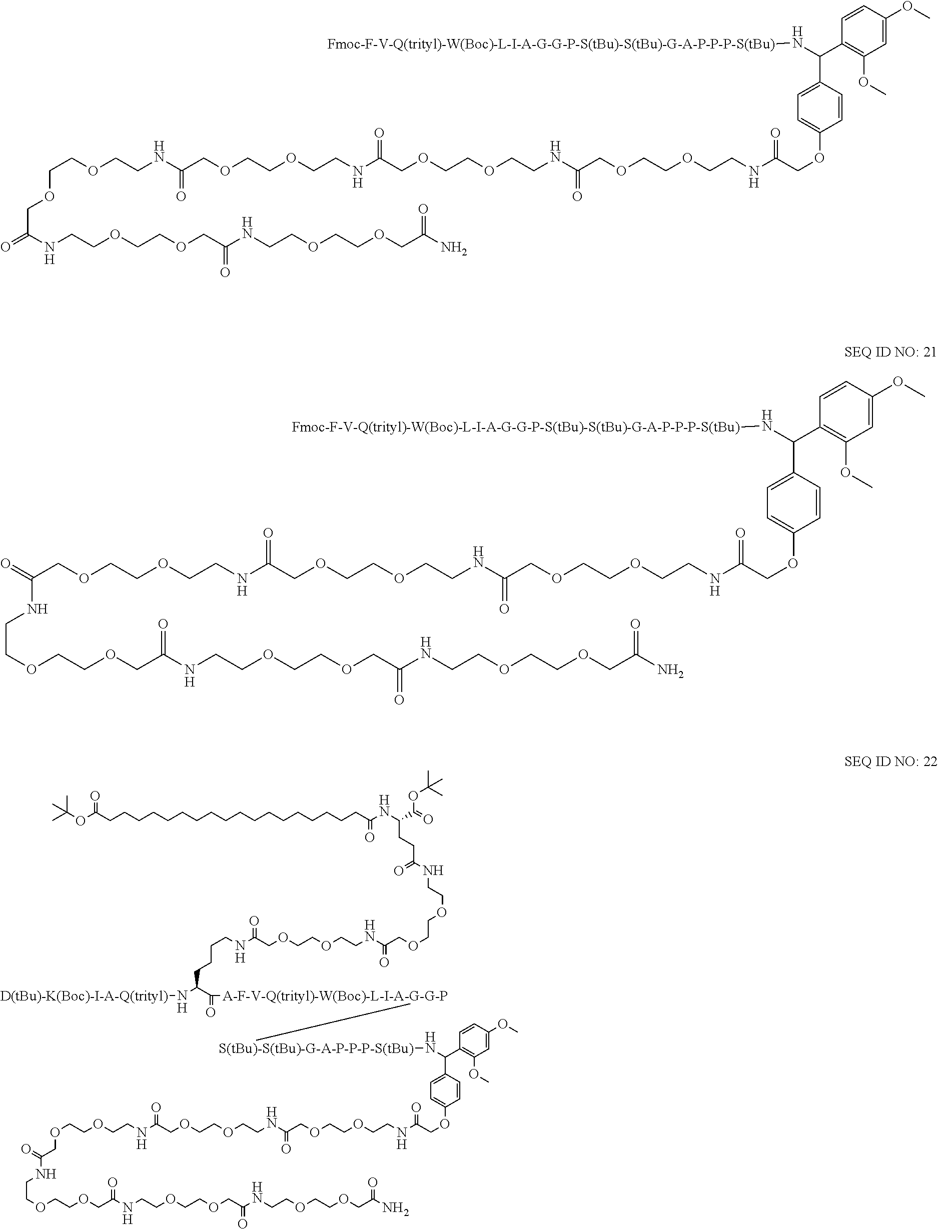

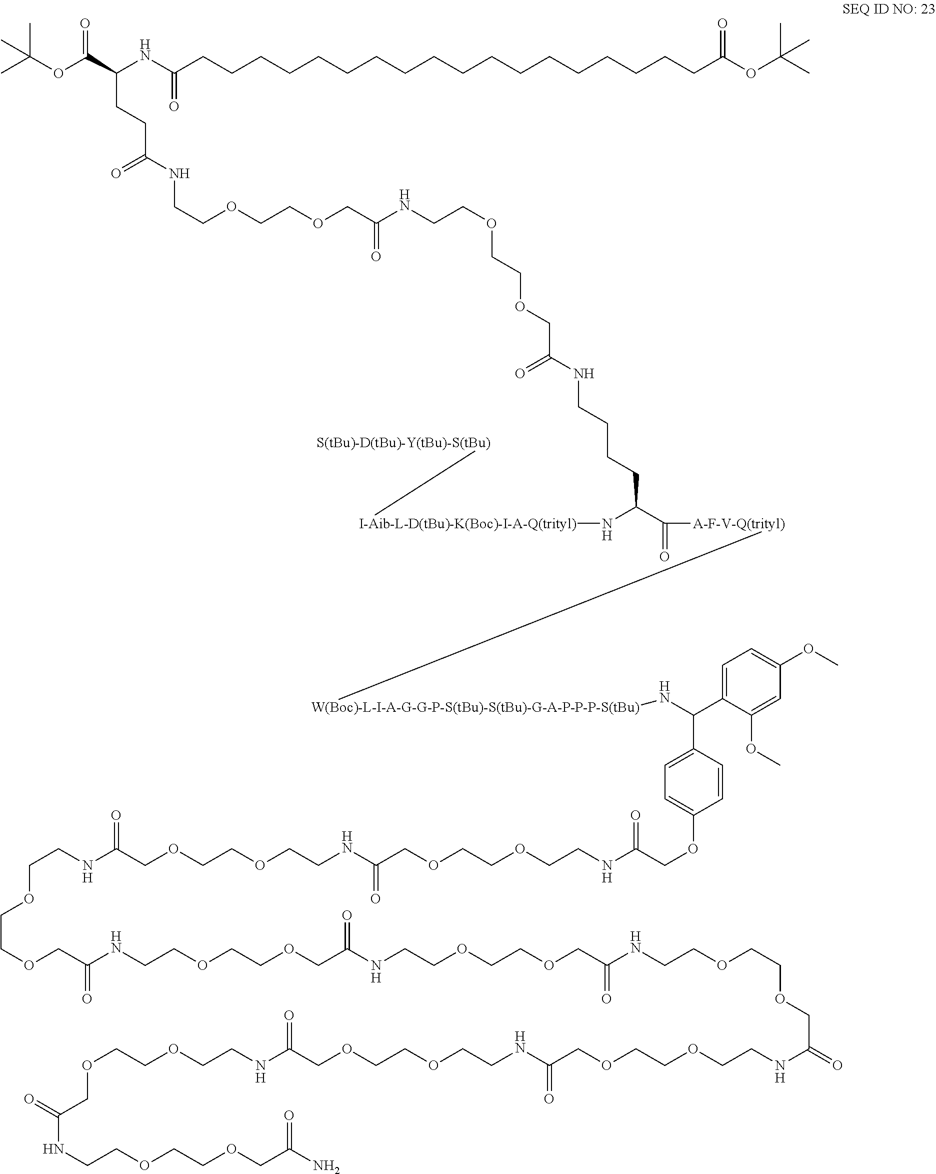

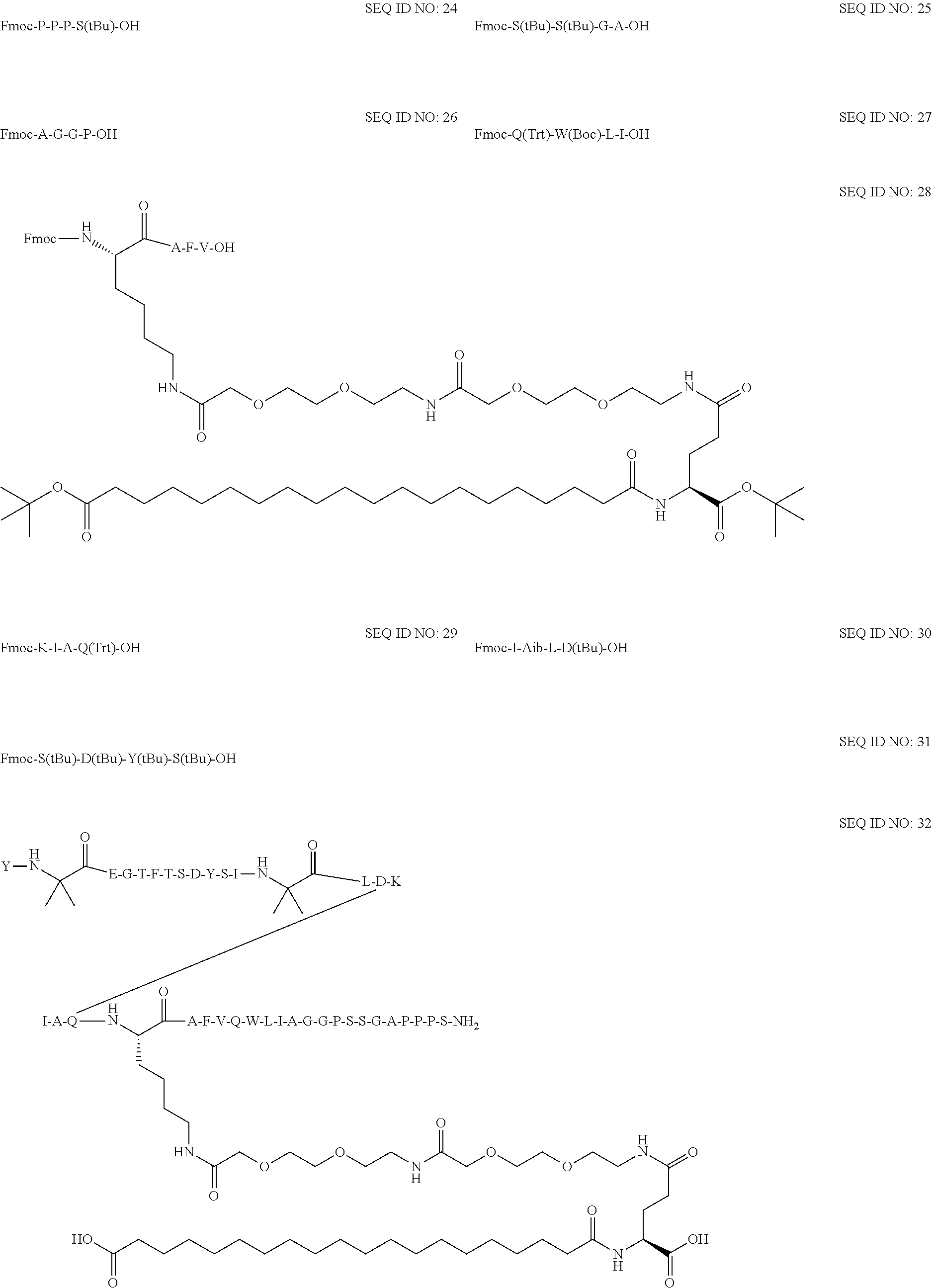

What is claimed is:

1. A compound of the formula 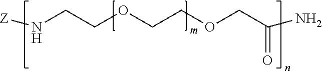
wherein “m” is 0 to 20, “n” is 1 to 50, and “Z” is a linker compound.

2. The compound of claim 1, wherein Z is selected from the group consisting of:

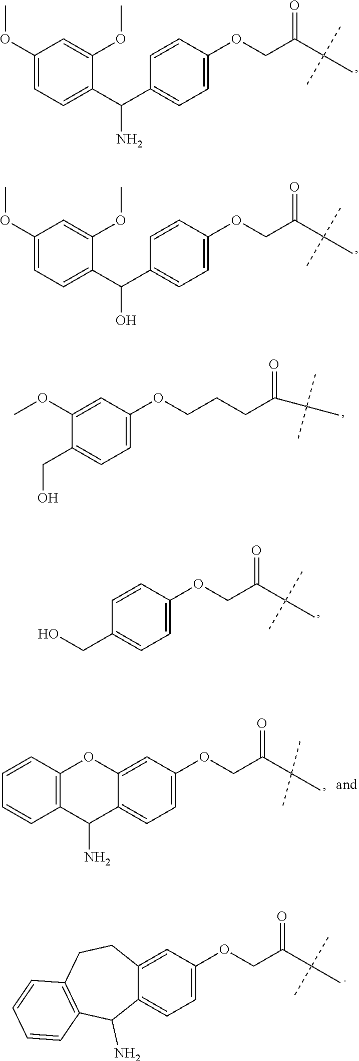

3. The compound of claim 2, wherein the compound is

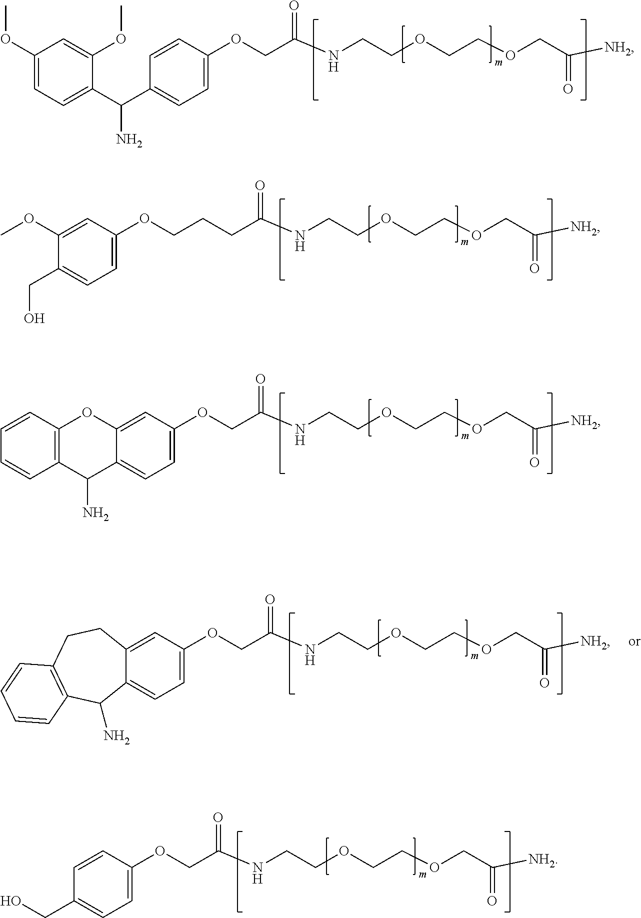

4.-7. (canceled)

8. The compound of claim 1, wherein
a. “m” is 0, 1, 2, or 3, and “n” is 1 to 50,
b. “m” is 0, 1, 2, or 3, and “n” is 1 to 10, or
c. “m” is 1 and “n” is 2, 3, 4, 5, 6, 7, 8, 9, or 10.

9.-10. (canceled)

11. The compound of claim 1, wherein the compound is used in liquid phase synthesis.

12. The compound of claim 1, wherein the liquid phase synthesis is Liquid Phase Peptide Synthesis (LPPS).

13. The compound of claim 1, wherein the compound is hydrophilic.

14. The compound of claim 1, wherein “Z” is a functional group which forms a covalent bond to an optionally protected amino acid, which can in turn undergo iterative deprotection and coupling steps onto one or more optionally protected amino acids or peptides, and then the resulting polypeptide product is able to be liberated from the “Z” group through chemical transformation.

15. A peptide of SEQ ID NO: 1, 3, 5, 6, 8, 9, 11, 12, 14, 15, 16, 17, 18, 19, 20, 21, 22 or 23.

16. A method of preparation of the peptide of:
a) SEQ ID NO: 2, comprising preparing the peptide of SEQ ID NO: 1 using liquid-phase peptide synthesis, and then treating the prepared peptide of SEQ ID NO: 1 with an acid to give the peptide of SEQ ID NO: 2;
b) SEQ ID NO: 4, comprising preparing the peptide of SEQ ID NO: 3 using liquid-phase peptide synthesis, and then treating the prepared peptide of SEQ ID NO: 3 with an acid to give the peptide of SEQ ID NO: 4;
c) SEQ ID NO: 7, comprising preparing the peptide of SEQ ID NO: 6 using liquid-phase peptide synthesis, and then treating the prepared peptide of SEQ ID NO: 6 with an acid to give the peptide of SEQ ID NO: 7;
d) SEQ ID NO: 10, comprising preparing the peptide of SEQ ID NO: 9 using liquid-phase peptide synthesis, and then treating the prepared peptide of SEQ ID NO: 9 with an acid to give the peptide of SEQ ID NO: 10;
e) SEQ ID NO: 13, comprising preparing the peptide of SEQ ID NO: 11, 12, 14, 15, 16, 17, or 18 using liquid-phase peptide synthesis, and then treating the prepared peptide of SEQ ID NO: 11, 12, 14, 15, 16, 17, or 18 with an acid to give the peptide of SEQ ID NO: 13;
f) SEQ ID NO: 20, comprising coupling the peptide of SEQ ID NO: 19 with the peptide of SEQ ID NO: 7; or
g) SEQ ID NO: 22, comprising coupling the peptide of SEQ ID NO: 21 with the peptide of SEQ ID NO: 10.

17.-37. (canceled)

38. A method for preparing a compound of the formula: 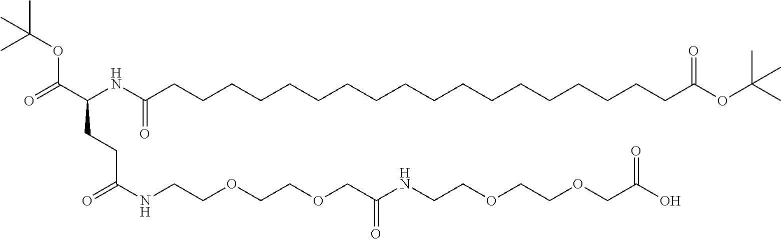 [SMILES: CC(C)(C)OC(=O)CCCCCCCCCCCCCCCCCCC(=O)N[C@@H](CCC(=O)NCCOCCOCC(=O)NCCOCCOCC(=O)O)C(=O)OC(C)(C)C]
comprising preparing the compound using liquid phase synthesis, wherein the liquid phase synthesis includes coupling an AEEA containing molecule to a compound of claim 1.

39. The method of claim 38, comprising:
a. coupling Fmoc-AEEA-OH onto a linker compound of claim 1 and deprotecting the resulting product using a base;
b. coupling a second Fmoc-AEEA-OH onto the product from step a. and deprotecting the resulting product using a base;
c. coupling Fmoc-Glu-OtBu onto the product of step b. and deprotecting the resulting product using a base;
d. coupling 20-(tert-butoxy)-20-oxoicosanoic acid onto the product of step c.; and
e. removing the linker compound from the product of step d. with an acid.

40. The method of claim 38, comprising:
a. coupling Fmoc-(AEEA)₂-OH onto a linker compound of claim 1 and deprotecting the resulting product using a base;
b. coupling Fmoc-Glu-OtBu onto the product of step a. and deprotecting the resulting product using a base;
c. coupling 20-(tert-butoxy)-20-oxoicosanoic acid onto the resulting product of step b.; and
d. removing the linker compound from the product of step c. with an acid.

41. The method of claim 38, comprising:
a. coupling Fmoc-(AEEA)₂-OH onto the linker compound of claim 1 and deprotecting the resulting product using a base;
b. coupling O1-tert-butyl O5-(2,5-dioxopyrrolidin-1-yl)(2S)-2-[(20-tert-butoxy-20-oxo-icosanoyl)amino]pentanedioate onto the resulting product of step a.; and
c. removing the linker compound from the product of step b. with an acid.

42. The compound which is

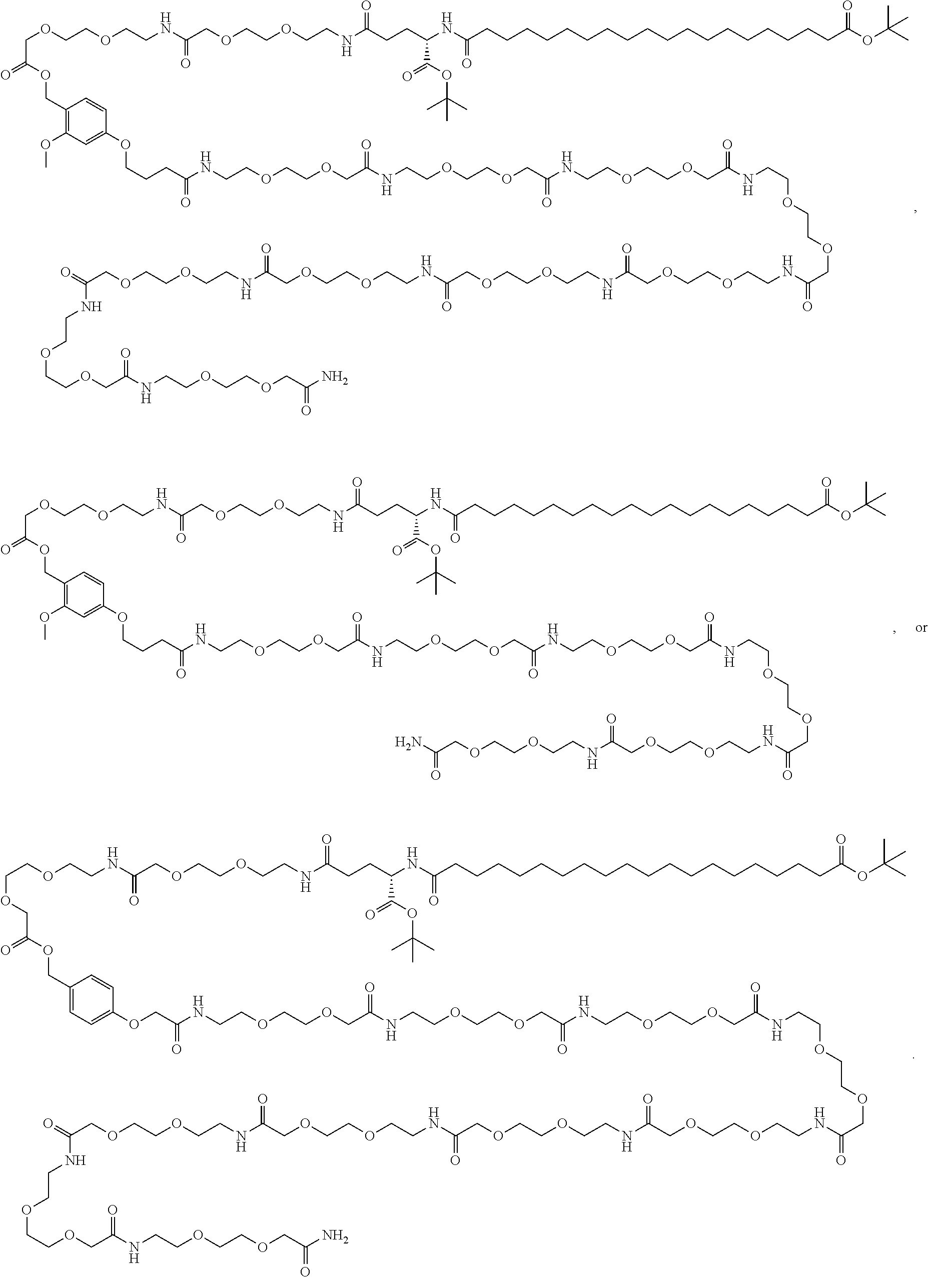

43.-44. (canceled)

45. A method for preparing the peptide of SEQ ID NO: 32, comprising preparing part or all of the peptide sequence by liquid-phase peptide synthesis using a compound according to claim 1.

46.-47. (canceled)

48. A method for preparing the peptide of SEQ ID NO: 32, comprising the following steps:
a. preparing the peptide of SEQ ID NO: 19 by liquid-phase peptide synthesis using a compound according to claim 1;
b. coupling the peptide of SEQ ID NO: 19 to the peptide of SEQ ID NO: 7 to generate the peptide of SEQ ID NO: 20;
c. deprotecting the peptide of SEQ ID NO: 20 to generate the peptide of SEQ ID NO: 21;
d. coupling the peptide of SEQ ID NO: 10 to the peptide of SEQ ID NO: 21; and
e. deprotecting the peptide from step d. to generate the peptide of SEQ ID NO: 22.

49. The method of claim 48, further comprising:
f. elongating the peptide of SEQ ID NO: 22 from step e. by coupling individual amino acids, peptide fragments, or mixtures thereof;
g. treating the resulting peptide with an acid; and
h deprotecting the resulting peptide.

50. A method for preparing the peptide of SEQ ID NO: 32 comprising the following steps:
a. coupling the peptide of SEQ ID NO: 24 to a compound according to claim 1 and deprotecting the resulting peptide;
b. coupling the peptide from step a. to the peptide of SEQ ID NO: 25 and deprotecting the resulting peptide;
c. coupling the peptide from step b. with the peptide of SEQ ID NO: 26 and deprotecting the resulting peptide;
d. coupling the peptide from step c. with the peptide of SEQ ID NO: 27 and deprotecting the resulting peptide;
e. coupling the peptide from step d. with the peptide of SEQ ID NO: 28 and deprotecting the resulting peptide;
f. coupling the peptide from step e. with the peptide of SEQ ID NO: 29 and deprotecting the resulting peptide;
g. coupling the peptide from step f. with the peptide of SEQ ID NO: 30 and deprotecting the resulting peptide; and
h. coupling the peptide of step g. with the peptide of SEQ ID NO: 31 and deprotecting the resulting peptide.
